(19) United States
(12) Patent Application Publication
(10) Pub. No.: US 2011/0318690 A1
(43) Pub. Date: Dec. 29, 2011

(54) COMPOUND, RESIN AND PHOTORESIST COMPOSITION

(75) Inventors: Yuko YAMASHITA, Osaka (JP); Hyungjoo KIM, Osaka (JP)

(73) Assignee: SUMITOMO CHEMICAL COMPANY, LIMITED, Tokyo (JP)

(21) Appl. No.: 13/168,480

(22) Filed: Jun. 24, 2011

(30) Foreign Application Priority Data
Jun. 29, 2010 (JP) 2010-147345

(51) Int. Cl. G03F 7/38; C08F 20/38; C08F 20/30; C07D 333/16; C07C 69/66; C07D 311/02; C07D 327/06; C08F 28/06; G03F 7/004

(52) U.S. Cl. 430/285.1; 526/257; 526/256; 526/268; 430/325; 560/220; 549/399; 560/221; 549/14; 549/78

(57) ABSTRACT

The present invention provides a compound represented by the formula (I):

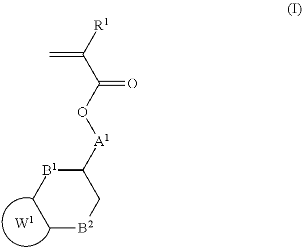

wherein R¹ represents a hydrogen atom or a methyl group, A¹ represents a single bond or *—(CH₂)ₘ—CO—O— in which m represents an integer of 1 to 4 and * represents a binding position to —O—, B¹ represents —O— or —S—, B² represents —CH₂—, —O— or —S— and W¹ represents an optionally substituted aromatic ring,
a resin comprising a structural unit derived from the compound and a photoresist composition comprising the resin.

This nonprovisional application claims priority under 35 U.S.C. §119(a) on Patent Application No. 2010-147345 filed in JAPAN on Jun. 29, 2010, the entire contents of which are hereby incorporated by reference.

### FIELD OF THE INVENTION

The present invention relates to a novel compound, a resin comprising a structural unit derived from the compound and a photoresist composition comprising the resin.

### BACKGROUND OF THE INVENTION

JP 2005-274877 A discloses a resin comprising a structural unit derived from 2-ethyl-2-adamantyl methacrylate and a structural unit derived from p-hydroxystyrene and a photoresist composition comprising the resin.

### SUMMARY OF THE INVENTION

The present invention is to provide a photoresist composition.

The present invention relates to the followings:

<1> A compound represented by the formula (I):

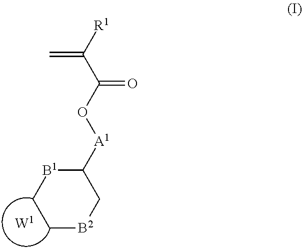

wherein R¹ represents a hydrogen atom or a methyl group, A¹ represents a single bond or *—(CH₂)ₘ—CO—O— in which m represents an integer of 1 to 4 and * represents a binding position to —O—, B¹ represents —O— or —S—, B² represents —CH₂—, —O— or —S— and W¹ represents an optionally substituted aromatic ring.
<2> A resin comprising a structural unit derived from the compound according to <1>;
<3> A photoresist composition comprising the resin according to <2>;
<4> The photoresist composition according to <3>, wherein the photoresist composition further contains an acid generator;
<5> The photoresist composition according to <3> or <4>, wherein the photoresist composition further contains a basic compound;
<6> A process for producing a photoresist pattern comprising the following steps (1) to (5):

(1) a step of applying the photoresist composition according to any one of <3> to <5> on a substrate,

(2) a step of forming a photoresist film by conducting drying,

(3) a step of exposing the photoresist film to radiation,

(4) a step of baking the exposed photoresist film, and

(5) a step of developing the baked photoresist film with an alkaline developer, thereby forming a photoresist pattern.

### DESCRIPTION OF PREFERRED EMBODIMENTS

The compound of the present invention is a compound represented by the formula (I):

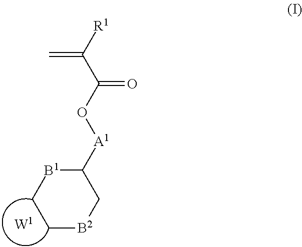

wherein R¹ represents a hydrogen atom or a methyl group, A¹ represents a single bond or *—(CH₂)ₘ—CO—O— in which m represents an integer of 1 to 4 and * represents a binding position to —O—, B¹ represents —O— or —S—, B² represents —CH₂—, —O— or —S— and W¹ represents an optionally substituted aromatic ring (hereinafter, simply referred to as the compound (I)).

The aromatic ring represented by W¹ usually has 6 to 14, preferably 6 to 10 and more preferably 6.

Examples of the aromatic ring include the following.

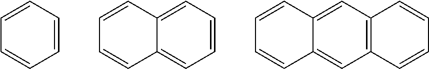

The aromatic ring may have one or more substituents, and examples of the substituents include a C1-C6 alkyl group, a C1-C6 alkoxy group and a C3-C6 cycloalkyl group. Examples of the C1-C6 alkyl group include a methyl group, an ethyl group, a propyl group, an isopropyl group, a butyl group, a sec-butyl group, a tert-butyl group, a pentyl group and a hexyl group. Examples of the C1-C6 alkoxy group include a methoxy group, an ethoxy group, a propoxy group, an isopropoxy group, a butoxy group, a sec-butoxy group, a tert-butoxy group, a pentyloxy group and a hexyloxy group. Examples of the C3-C6 cycloalkyl group include a cyclopropyl group, a cyclobutyl group, a cyclopentyl group and a cyclohexyl group.

The compound (I) is preferably a compound represented by the formula (II):

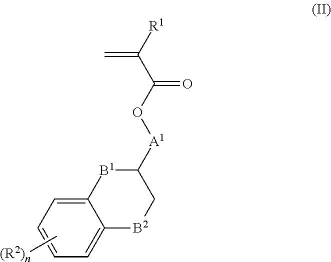

wherein R¹, A¹, B¹ and B² are the same as defined above, and R² represents a C1-C6 alkyl group, a C1-C6 alkoxy group or a C3-C6 cycloalkyl group and n represents 0 or 1.

Examples of the compound (I) include the following.

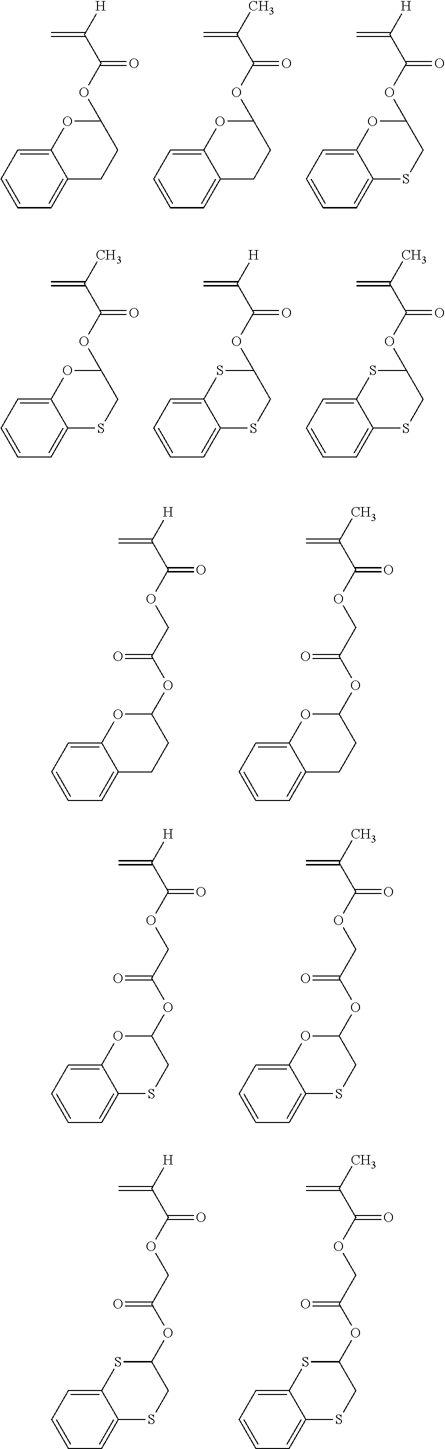

The compound (I) can be produced by reacting acrylic halide or methacrylic halide with the corresponding cyclic alcohol having an optionally substituted aromatic ring in the presence of a base.

The reaction preferably conducted at −10 to 10° C. The reaction preferably conducted in a solvent such as tetrahydrofuran and N,N-dimethylformamide. Examples of the base include an organic base such as triethylamine. The compound (I) can be isolated by extracting the reaction mixture with an organic solvent such as ethyl acetate followed by concentrating the organic layer obtained.

The obtained compound (I) can be further purified with conventional purification means such as column chromatography.

The resin of the present invention comprises a structural unit derived from the compound (I). The resin of the present invention may contain one or more structural units derived from a monomer or monomers different from the compound (I) in addition to the structural unit derived from the compound (I).

In the resin having one or more structural units derived from a monomer or monomers different from the compound (I) in addition to the structural unit derived from the compound (I), the content of the structural unit derived from the compound (I) is usually 10 to 95% by mole, preferably 20 to 90% by mole and more preferably 30 to 80% by mole based on 100% by mole of all the structural units of the resin.

Examples of the monomers different from the compound (I) include a monomer having an acid-labile group and an acid-stable monomer having no acid-labile group.

The compound is a monomer having an acid-labile group.

The resin preferably contains a structural unit derived from an acid-stable monomer having no acid-labile group in addition to the structural unit derived from the compound (I).

The resin can be produced according to known polymerization method.

The resin of the present invention is itself insoluble or poorly soluble in an alkali aqueous solution but becomes soluble in an alkali aqueous solution by the action of an acid.

In this specification, “an acid-labile group” means a group capable of being eliminated by the action of an acid.

In the present specification, “ester group” means “a structure having ester of carboxylic acid”. Specifically, “tert-butyl ester group” is “a structure having tert-butyl ester of carboxylic acid”, and may be described as “—COOC(CH₃)₃”.

Examples of the acid-labile group include a structure having ester of carboxylic acid such as an alkyl ester group in which a carbon atom adjacent to the oxygen atom is quaternary carbon atom, an alicyclic ester group in which a carbon atom adjacent to the oxygen atom is quaternary carbon atom, and a lactone ester group in which a carbon atom adjacent to the oxygen atom is quaternary carbon atom. The “quaternary carbon atom” means a “carbon atom joined to four substituents other than hydrogen atom”.

Examples of the acid-labile group include a group represented by the formula (10):

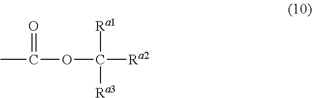

wherein R^{a1}, R^{a2} and R^{a3} independently represent a C1-C8 aliphatic hydrocarbon group or a C3-C20 saturated cyclic hydrocarbon group, and R^{a1} and R^{a2} can be bonded each other to form a C3-C20 ring together with the carbon atom to which they are bonded.

Examples of the C1-C8 aliphatic hydrocarbon group include a C1-C8 alkyl group. Specific examples of the C1-C8 alkyl group include a methyl group, an ethyl group, a propyl group, an isopropyl group, a butyl group, a pentyl group, a hexyl group, a heptyl group and an octyl group. The C3-C20 saturated cyclic hydrocarbon group may be monocyclic or polycyclic, and examples thereof include a monocyclic alicyclic hydrocarbon group such as a C3-C20 cycloalkyl group (e.g. a cyclopentyl group, a cyclohexyl group, a methylcyclohexyl group, a dimethylcyclohexyl group, a cycloheptyl group and a cyclooctyl group) and a polycyclic alicyclic hydrocarbon group such as a decahydronaphthyl group, an adamantyl group, a norbornyl group, a methylnorbornyl group, and the following:

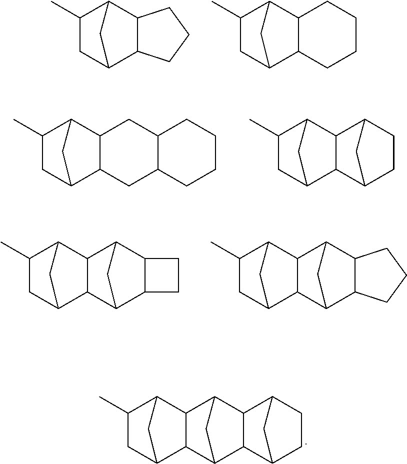

The saturated cyclic hydrocarbon group preferably has 5 to 16 carbon atoms.

Examples of the ring formed by bonding R^{a1} and R^{a2} each other together with the carbon atom to which they are bonded include the following groups and the ring preferably has 3 to 12 carbon atoms.

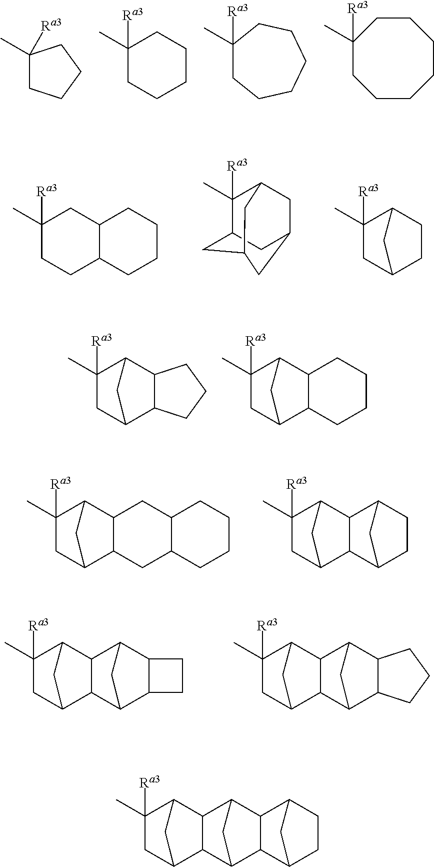

wherein R^{a3} is the same as defined above.

The group represented by the formula (10) wherein R^{a1}, R^{a2} and R^{a3} independently each represent a C1-C8 alkyl group such as a tert-butyl group, the group represented by the formula (10) wherein R^{a1} and R^{a2} are bonded each other to form an adamantyl ring and R^{a3} is a C1-C8 alkyl group such as a 2-alkyl-2-adamantyl group, and the group represented by the formula (10) wherein R^{a1} and R^{a2} are C1-C8 alkyl groups and R^{a3} is an adamantyl group such as a 1-(1-adamantyl)-1-alkylalkoxycarbonyl group are preferable.

The monomer having an acid-labile group (hereinafter, simply referred to as the monomer (a1)) is preferably a monomer having the acid-labile group represented by the formula (10) and a carbon-carbon double bond, and more preferably an acrylate monomer having an acid-labile group represented by the formula (10) in its side chain or a methacrylate monomer having an acid-labile group represented by the formula (10) in its side chain.

Preferable examples of the monomer (a1) include a monomer having a C5-C20 saturated cyclic hydrocarbon group. When the photoresist composition contains a resin derived from a monomer having a bulky structure such as a saturated cyclic hydrocarbon group, the photoresist composition having excellent resolution tends to be obtained.

Preferable examples of the monomer (a1) include a monomer represented by the formula (a1-1) and a monomer represented by the formula (a1-2):

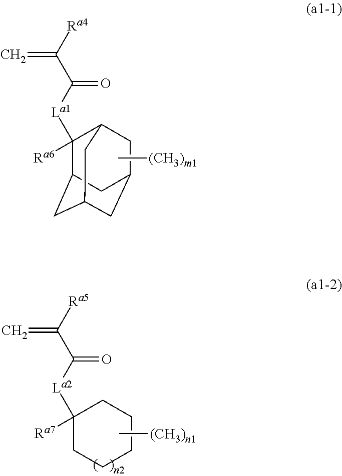

wherein R^{a4} and R^{a5} independently represents a hydrogen atom or a methyl group, R^{a6} and R^{a7} independently represents a C1-C8 aliphatic hydrocarbon group or a C3-C10 saturated cyclic hydrocarbon group, L^{a1} and L^{a2} independently represents *—O— or *—O—(CH₂)ₖ₁—CO—O— in which * represents a binding position to —CO—, and k1 represents an integer of 1 to 7, m1 represents an integer of 0 to 14, n1 represents an integer of 0 to 10 and n2 represents 0 or 1.

R^{a4} and R^{a5} are preferably methyl groups.

The aliphatic hydrocarbon group preferably has 1 to 6 carbon atoms, and the saturated cyclic hydrocarbon group preferably has 3 to 8 carbon atoms and more preferably 3 to 6 carbon atoms.

Examples of the aliphatic hydrocarbon group include a C1-C8 alkyl group such as a methyl group, an ethyl group, a propyl group, an isopropyl group, a butyl group, a tert-butyl group, a pentyl group, a hexyl group, a heptyl group and an octyl group. The saturated cyclic hydrocarbon group may be monocyclic or polycyclic. Examples of the saturated monocyclic hydrocarbon group include a cycloalkyl group such as a cyclohexyl group, a methylcyclohexyl group, a dimethylcyclohexyl group, a cycloheptyl group, a methylcycloheptyl group, and examples of the saturated polycyclic hydrocarbon group include a decahydronaphthyl group, an adamantyl group, a norbornyl group, a methylnorbornyl group and the following:

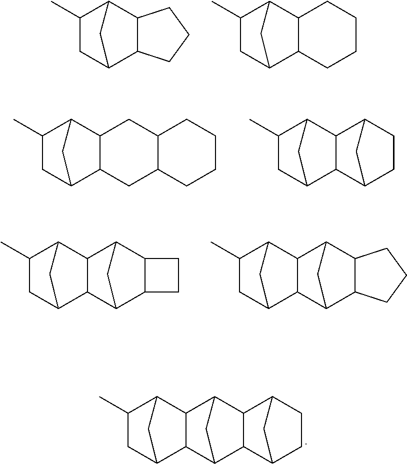

L^{a1} is preferably *—O— or *—O— (CH₂)_{f1}—CO—O— in which * represents a binding position to —CO—, and f1 represents an integer of 1 to 4, and is more preferably *—O— or *—O—CH₂—CO—O—, and is especially preferably *—O—. L^{a2} is preferably *—O— or *—O—(CH₂)—CO—O— in which * represents a binding position to —CO—, and f1 is the same as defined above, and is more preferably *—O— or *—O—CH₂—CO—O—, and is especially preferably *—O—.

In the formula (a1-1), m1 is preferably an integer of 0 to 3, and is more preferably 0 or 1. In the formula (a1-2), n1 is preferably an integer of 0 to 3, and is more preferably 0 or 1.

Examples of the monomer represented by the formula (a1-1) include the following.

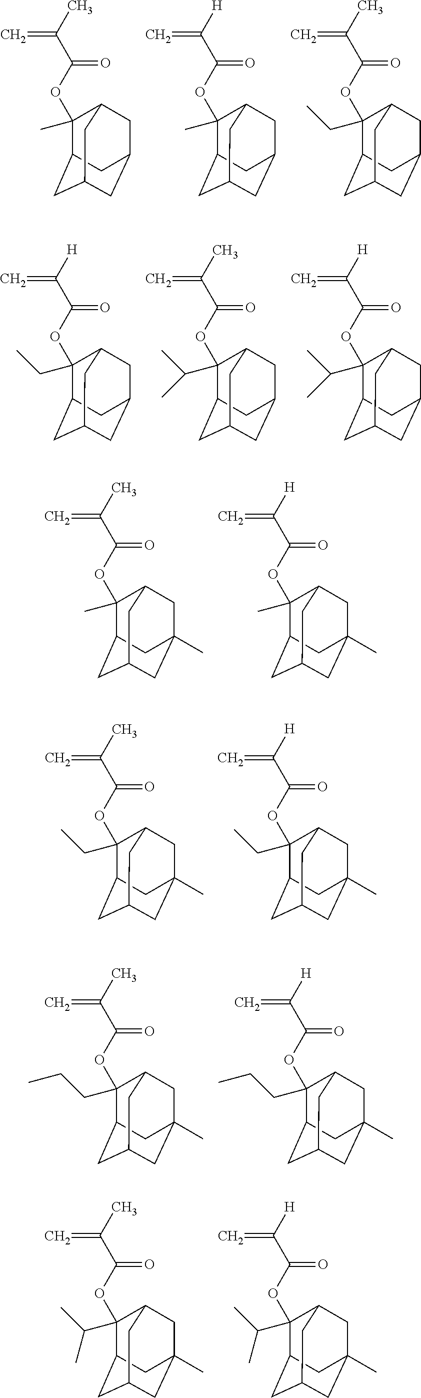

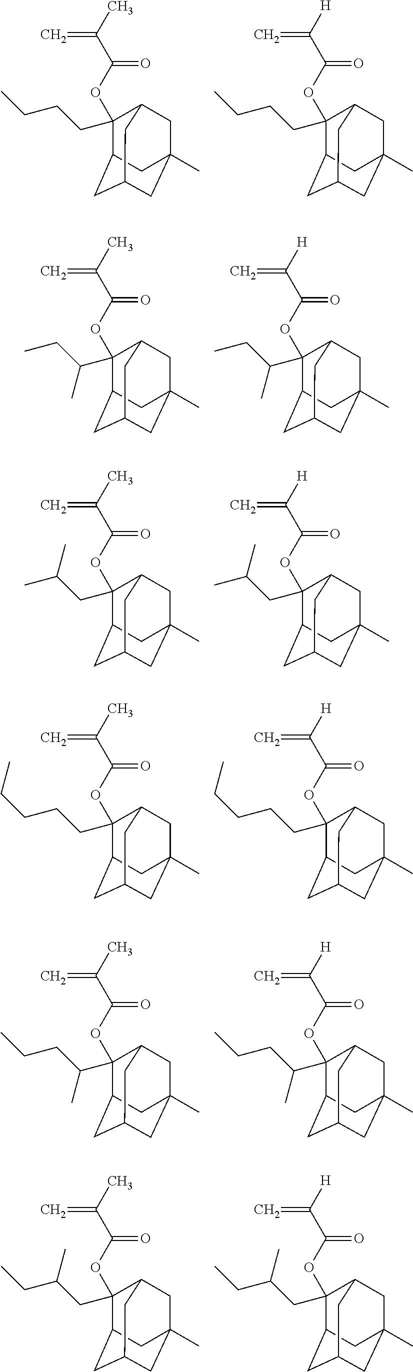

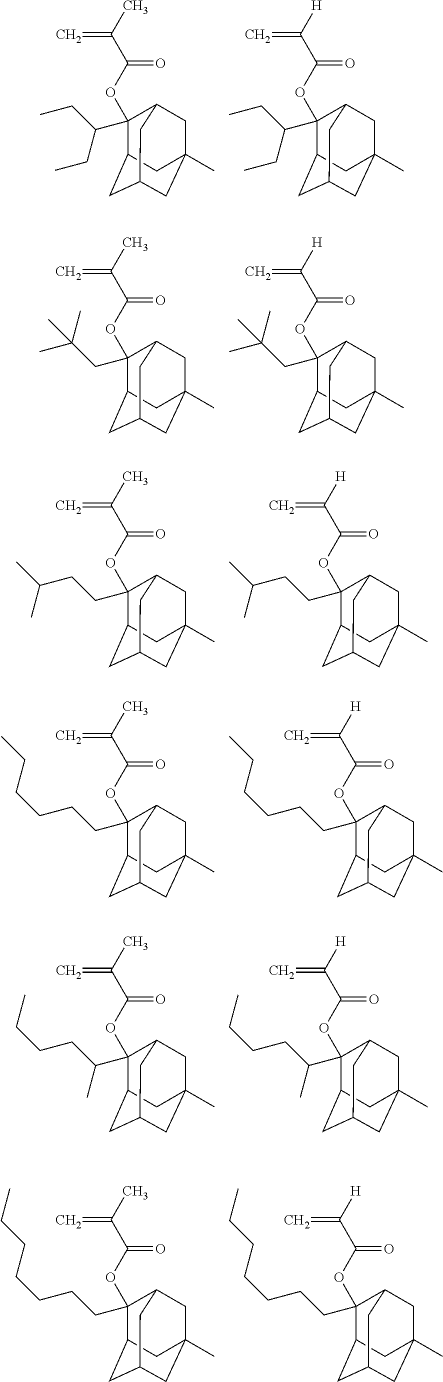

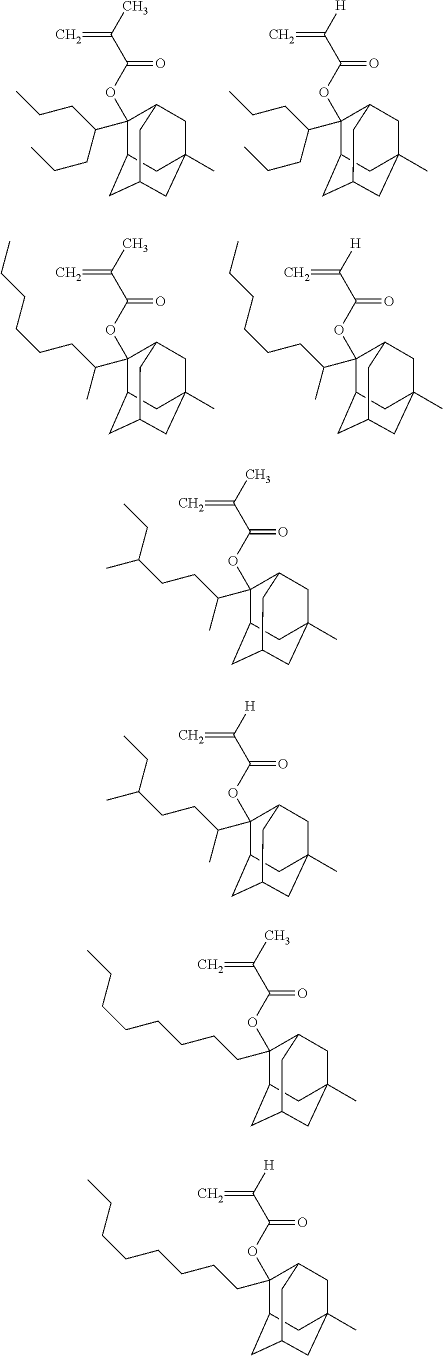

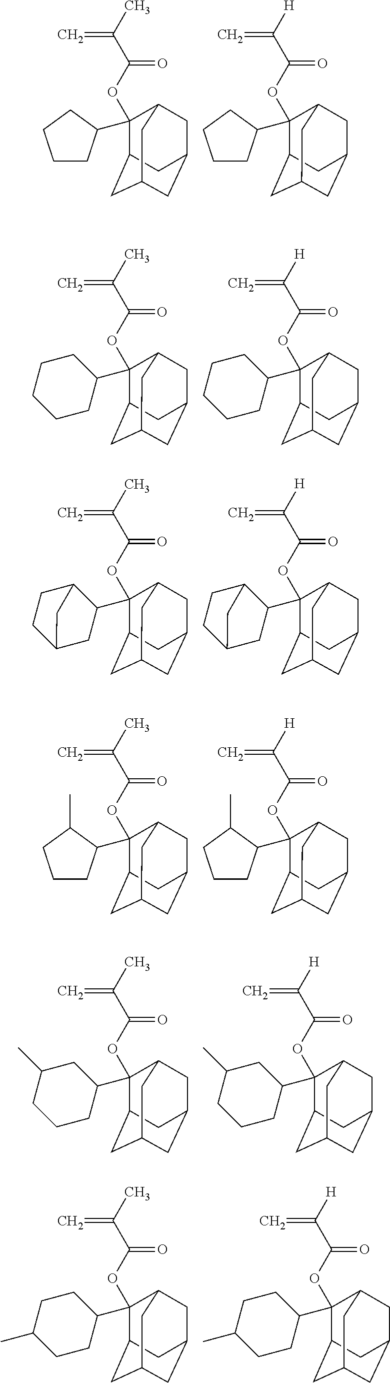

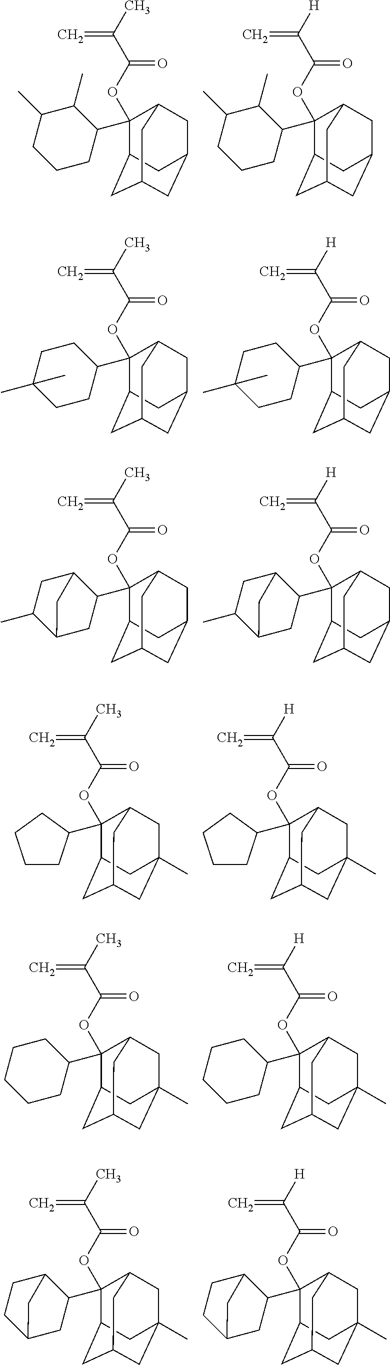

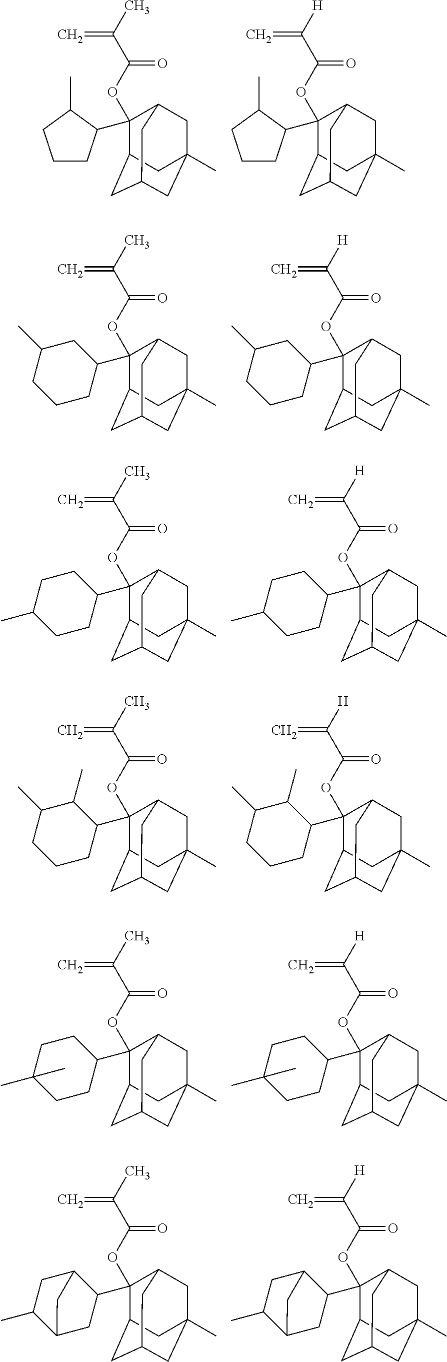

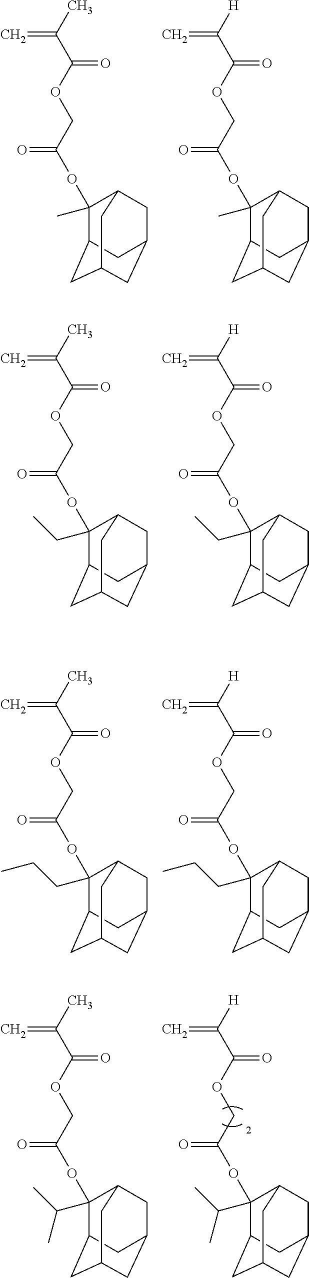

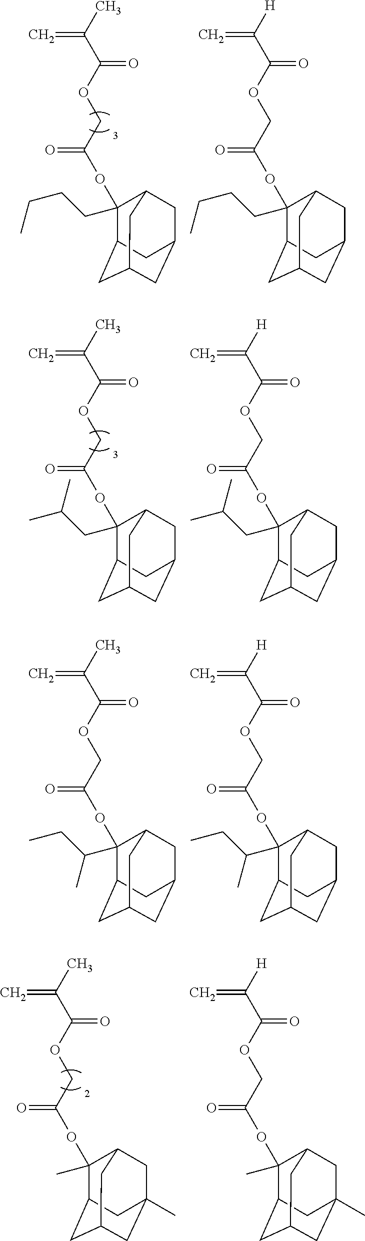

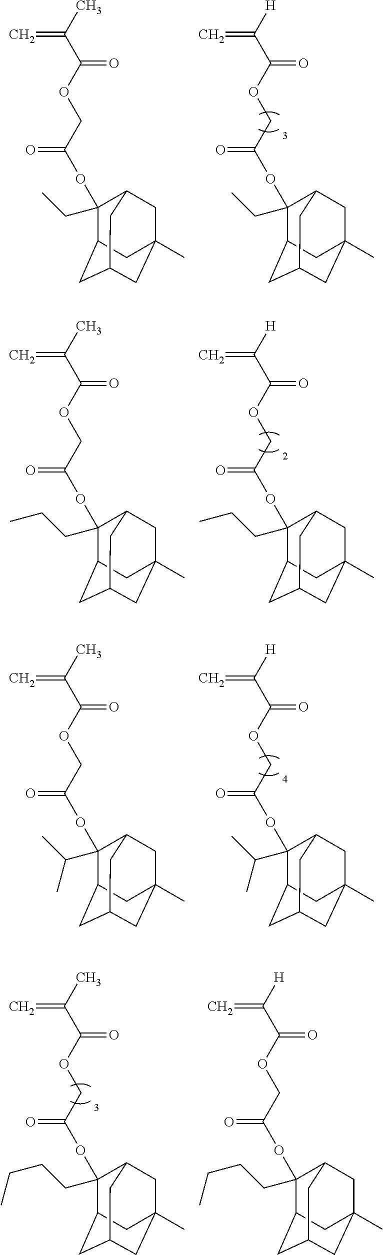

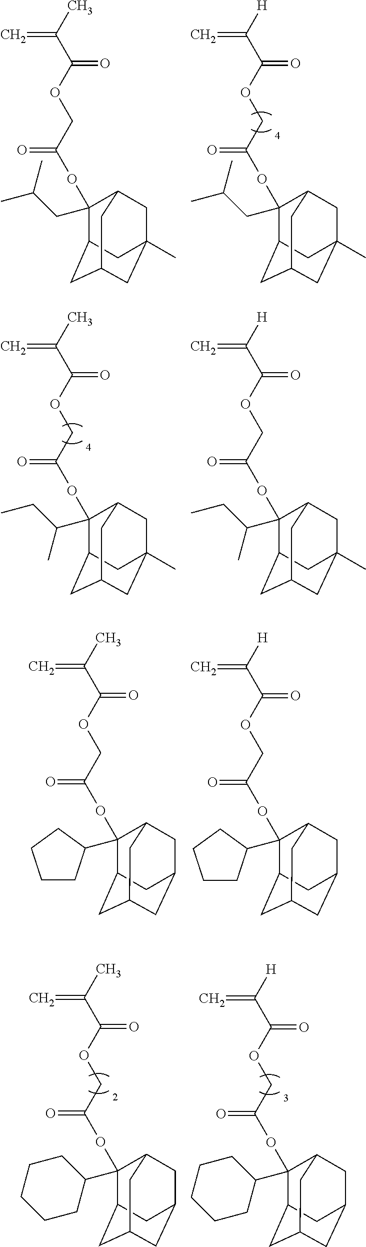

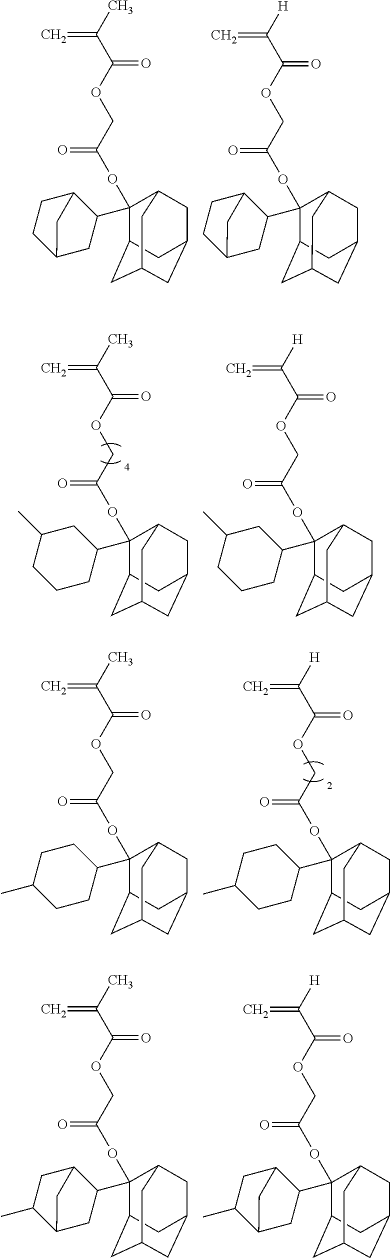

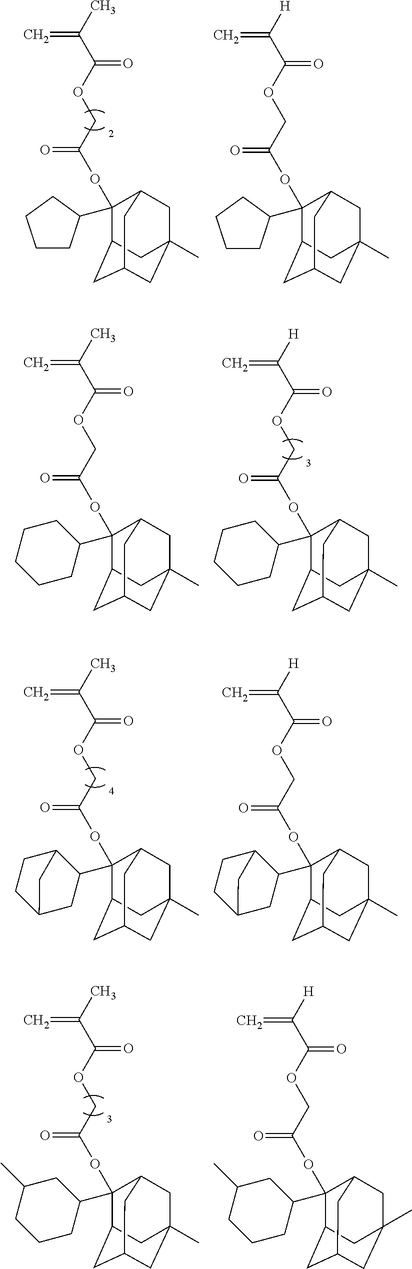

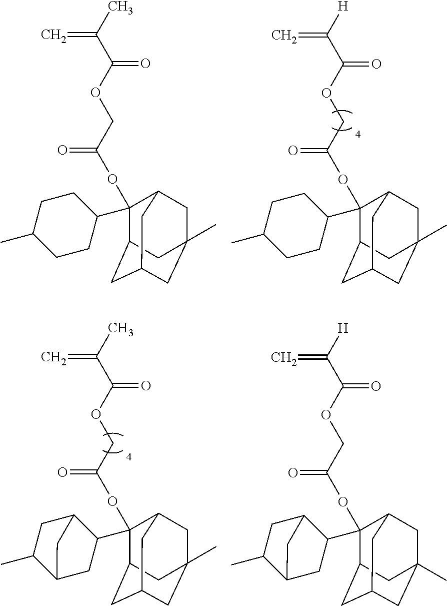

Among them, preferred are 2-methyl-2-adamantyl acrylate, 2-methyl-2-adamantyl methacrylate, 2-ethyl-2-adamantyl acrylate, 2-ethyl-2-adamantyl methacrylate, 2-isopropyl-2-adamantyl acrylate and 2-isopropyl-2-adamantyl methacrylate, and more preferred are 2-methyl-2-adamantyl methacrylate, 2-ethyl-2-adamantyl methacrylate, and 2-isopropyl-2-adamantyl methacrylate.

Examples of the monomer represented by the formula (a1-2) include the following.

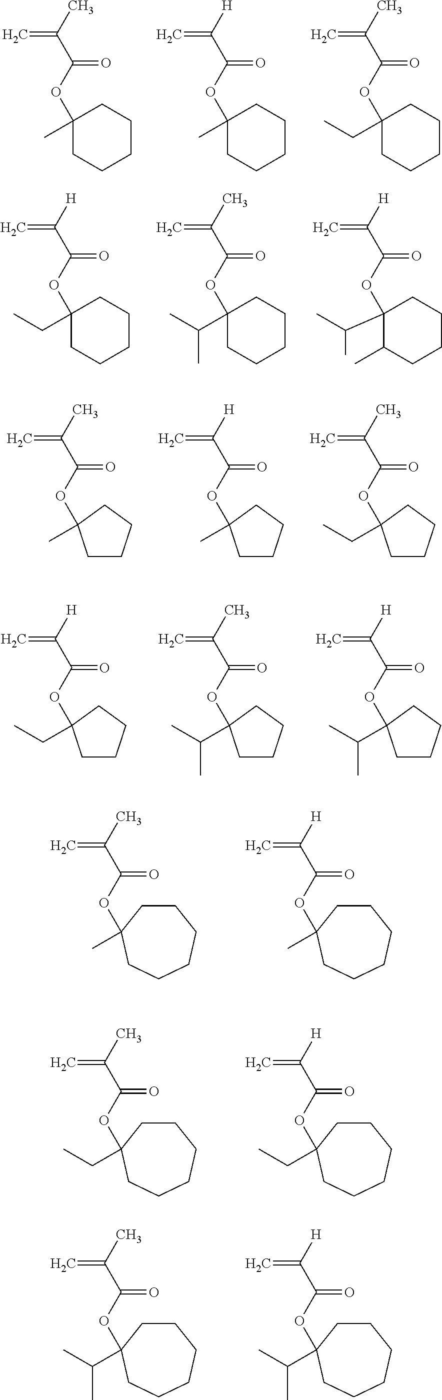

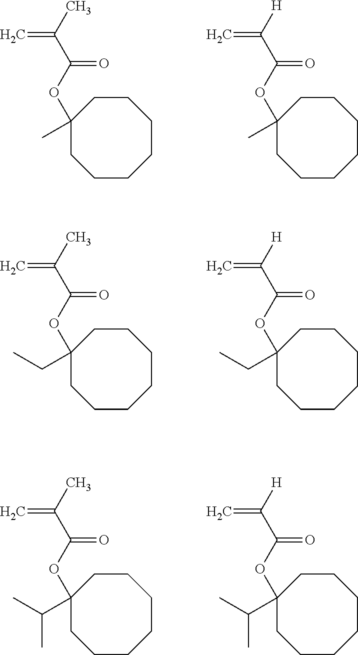

Among them, preferred are 1-ethyl-1-cyclohexyl acrylate, 1-ethyl-1-cyclohexyl methacrylate, 1-ethyl-1-cyclopentyl acrylate and 1-ethyl-1-cyclopentyl methacrylate, and more preferred are 1-ethyl-1-cyclohexyl methacrylate and 1-ethyl-1-cyclopentyl methacrylate.

The content of the structural unit derived from the monomer (a1) in the resin is usually 10 to 95% by mole, preferably 15 to 90% by mole and more preferably 20 to 85% by mole based on 100% by mole of all the structural units of the resin.

The resin can have two or more kinds of structural units derived from the monomer (a1).

The resin preferably contains the structural unit derived from the acid-stable monomer having no acid-labile group.

The acid-stable monomer having no acid-labile group preferably contains one or more hydroxyl groups or a lactone ring.

When the resin contains the structural unit derived from the acid-stable monomer having no acid-labile group and having one or more hydroxyl groups or a lactone ring, a photoresist composition having good resolution and adhesiveness of photoresist to a substrate tends to be obtained.

When the photoresist composition of the present invention is used for KrF excimer laser (wavelength: 248 nm) lithography, EUV lithography and EB lithography, the resin of the present invention preferably contains a structural unit derived from the acid-stable monomer having no acid-labile group and having one or more phenolic hydroxyl groups. The resin can have two or more kinds of the structural unit derived from the acid-stable monomer having no acid-labile group and having one or more phenolic hydroxyl groups.

Examples of the acid-stable monomer having no acid-labile group and having one or more phenolic hydroxyl groups include a monomer represented by the formula (a2-0):

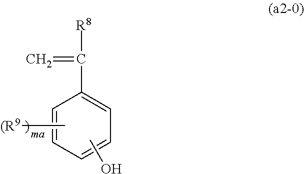

wherein R⁸ represents a hydrogen atom, a halogen atom, a C1-C6 alkyl group or a C1-C6 halogenated alkyl group, R⁹ is independently in each occurrence a halogen atom, a hydroxyl group, a C1-C6 alkyl group, a C1-C6 alkoxy group, a C2-C4 acyl group, a C2-C4 acyloxy group, an acryloyl group or a methacryloyl group, ma represents an integer of 0 to 4.

In the formula (a2-0), examples of the halogen atom include a fluorine atom. Examples of the C1-C6 alkyl group include a methyl group, an ethyl group, a propyl group, an isopropyl group, a butyl group, an isobutyl group, a sec-butyl group, a tert-butyl group, a pentyl group and a hexyl group, and a C1-C4 alkyl group is preferable and a C1-C2 alkyl group is more preferable and a methyl group is especially preferable. Examples of the C1-C6 alkoxy group include a methoxy group, an ethoxy group, a propoxy group, an isopropoxy group, a butoxy group, an isobutoxy group, a sec-butoxy group, a tert-butoxy group, a pentyloxy group and a hexyloxy group, and a C1-C4 alkoxy group is preferable and a C1-C2 alkoxy group is more preferable and a methoxy group is especially preferable. Examples of the C2-C4 acyl group include an acetyl group, a propionyl group and a butyryl group, and examples of the C2-C4 acyloxy group include an acetyloxy group, a propionyloxy group and a butyryloxy group. In the formula (a2-0), ma is preferably 0, 1 or 2, and is more preferably 0 or 1, and especially preferably 0.

The resin containing the structural unit derived from the monomer represented by the formula (a2-0) can be produced, for example, by polymerizing a monomer obtained by protecting a hydroxyl group of the monomer represented by the formula (a2-0) with an acetyl group with other monomers followed by conducting deacetylation of the obtained polymer with a base.

Examples of the monomer represented by the formula (a2-0) include the followings.

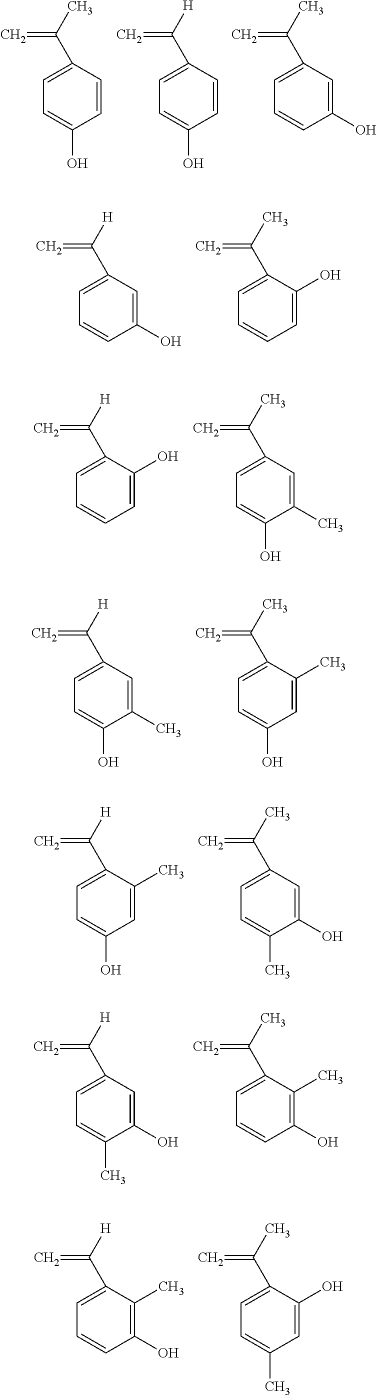

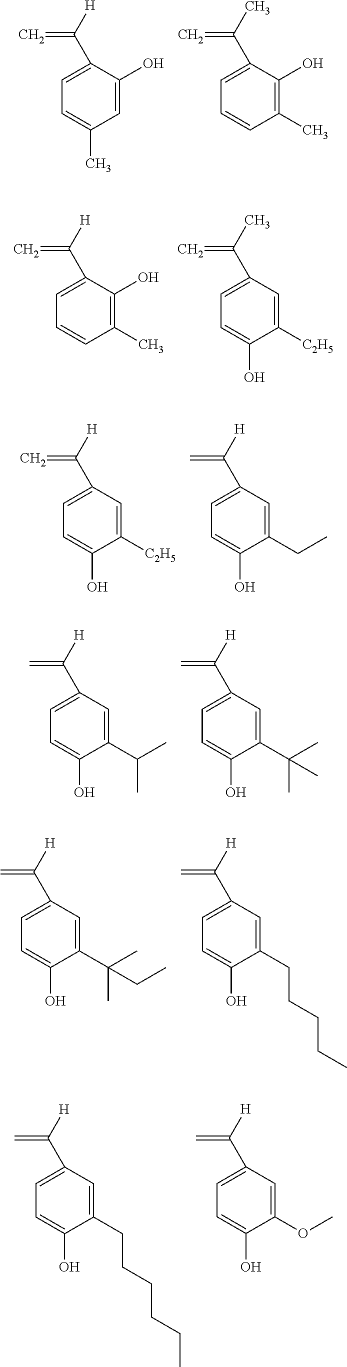

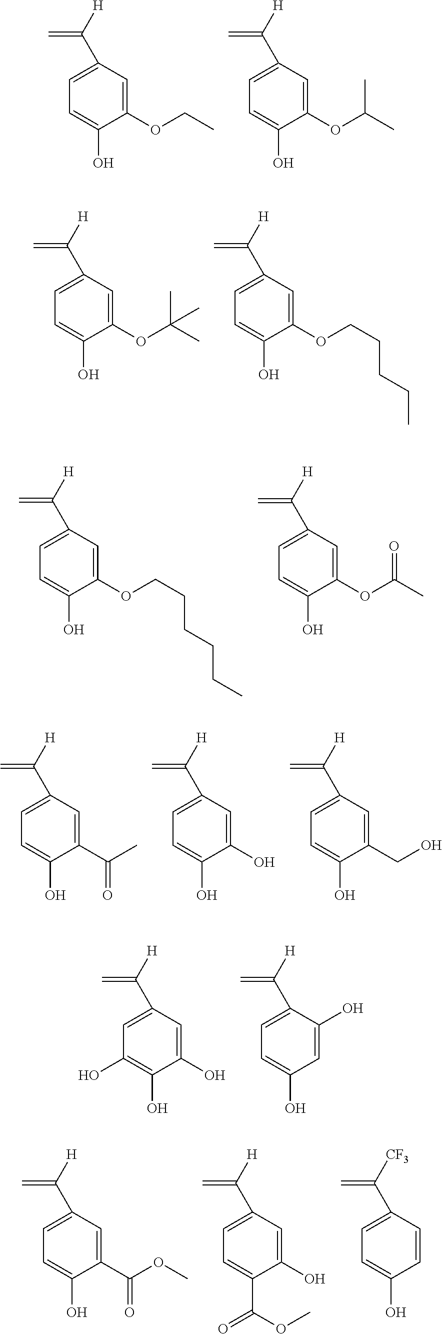

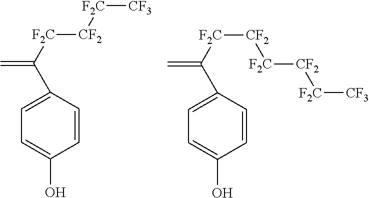

Among them, preferred are 4-hydroxystyrene and 4-hydroxy-α-methylstyrene.

When the resin contains the structural unit derived from the monomer represented by the formula (a2-0), the content of the structural unit derived from the monomer represented by the formula (a2-0) is usually 5 to 90% by mole and preferably 10 to 85% by mole and more preferably 15 to 80% by mole based on total molar of all the structural units of the resin.

Examples of the acid-stable monomer having no acid-labile group and having one or more phenolic hydroxyl groups include a monomer represented by the formula (a2-10):

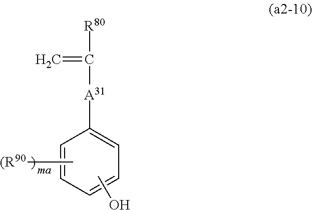

wherein R⁸⁰ represents a hydrogen atom, a halogen atom, a C1-C6 alkyl group or a C1-C6 halogenated alkyl group, R⁹⁰ is independently in each occurrence a halogen atom, a hydroxyl group, a C1-C6 alkyl group, a C1-C6 alkoxy group, a C2-C4 acyl group, a C2-C4 acyloxy group, an acryloyl group or a methacryloyl group, mb represents an integer of 0 to 4, and A³¹ represents a divalent connecting group.

In the formula (a2-10), examples of the halogen atom include a fluorine atom, examples of the C1-C6 alkyl group include a methyl group, an ethyl group, a propyl group, an isopropyl group, a butyl group, an isobutyl group, a sec-butyl group, a tert-butyl group, a pentyl group and a hexyl group, and a C1-C4 alkyl group is preferable and a C1-C2 alkyl group is more preferable and a methyl group is especially preferable. Examples of the C1-C6 halogenated alkyl group include a trifluoromethyl group, a pentafluoroethyl group, a heptafluoropropyl group, a heptafluoroisopropyl group, a nonafluorobutyl group, a nonafluoro-sec-butyl group, a nonafluoro-tert-butyl group, a perfluoropentyl group and a perfluorohexyl group. Examples of the C1-C6 alkoxy group include a methoxy group, an ethoxy group, a propoxy group, an isopropoxy group, a butoxy group, an isobutoxy group, a sec-butoxy group, a tert-butoxy group, a pentyloxy group and a hexyloxy group, and a C1-C4 alkoxy group is preferable and a C1-C2 alkoxy group is more preferable and a methoxy group is especially preferable. Examples of the C2-C4 acyl group include an acetyl group, a propionyl group and a butyryl group, and examples of the C2-C4 acyloxy group include an acetyloxy group, a propionyloxy group and a butyryloxy group. In the formula (a2-10), mb is preferably 0, 1 or 2, and is more preferably 0 or 1, and especially preferably 0.

The resin containing the structural unit derived from the monomer represented by the formula (a2-10) can be produced, for example, by polymerizing a monomer obtained by protecting a hydroxyl group of the monomer represented by the formula (a2-10) with an acetyl group followed by conducting deacetylation of the obtained polymer with a base.

Examples of the divalent connecting group include *—CO-T¹⁰-, and *—(CH₂)_{n′}-T¹¹- in which * represents a binding position to CH₂═C(R⁸⁰)—, T¹⁰ represents —O— or —NH—, T¹¹ represents a single bond, —O—, —CO—O— or —NH—CO—O— and n′ represents an integer of 0 to 4.

T¹⁰ is preferably —O—, and n′ is preferably 0, 1 or 2.

Specific examples of A³¹ include *—CO—O—, *—CO—NH—, *—CO—O—CH₂—CO—O—, *—CO—O—(CH₂)₂—O—, and *—CO—O—(CH₂)₂—NH—CO—O—.

Examples of the monomer represented by the formula (a2-10) include the followings.

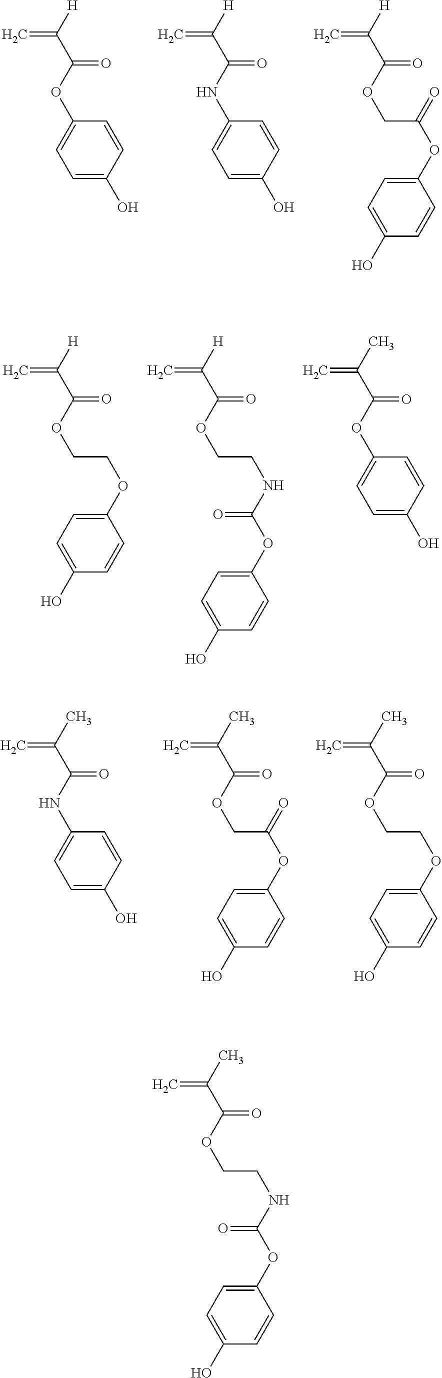

Among them, preferred is p-hydroxyphenyl methacrylate.

Examples of the acid-stable monomer having no acid-labile group and having one or more phenolic hydroxyl groups include a monomer represented by the formula (a2-20):

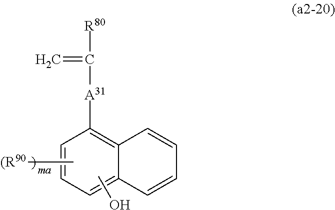

wherein R⁸⁰, R⁹⁰, mb and A³¹ are the same as defined above,

Examples of the monomer represented by the formula (a2-20) include the following:

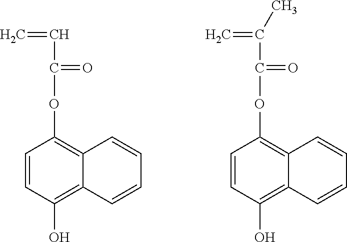

When the resin contains the structural unit derived from the monomer represented by the formula (a2-10) or (a2-20), the content of the structural unit derived from the monomer represented by the formula (a2-10) or (a2-20) is usually 5 to 90% by mole and preferably 10 to 80% by mole and more preferably 15 to 70% by mole based on total molar of all the structural units of the resin.

When the photoresist composition of the present invention is used for ArF excimer laser (wavelength: 193 nm) lithography, the resin of the present invention preferably contains a structural unit derived from the monomer represented by the formula (a2-1):

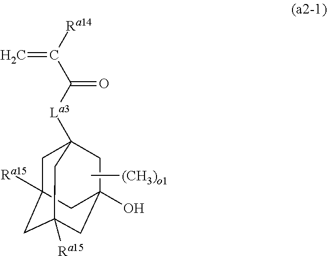

wherein R^{a14} represents a hydrogen atom or a methyl group, R^{a15} and R^{a16} independently represent a hydrogen atom, a methyl group or a hydroxyl group, L^{a3} represents *—O— or *—O— (CH₂)ₖ₂—CO—O— in which * represents a binding position to —CO—, and k2 represents an integer of 1 to 7, and of represents an integer of 0 to 10.

In the formula (a2-1), R^{a14} is preferably a methyl group, R^{a15} is preferably a hydrogen atom, R^{a16} is preferably a hydrogen atom or a hydroxyl group, L^{a3} is preferably *—O— or *—O— (CH₂)_{f1}—CO—O— in which * represents a binding position to —CO— and f1 represents an integer of 1 to 4, and is more preferably *—O—, and of is preferably 0, 1, 2 or 3 and is more preferably 0 or 1.

Examples of the monomer represented by the formula (a2-1) include the followings:

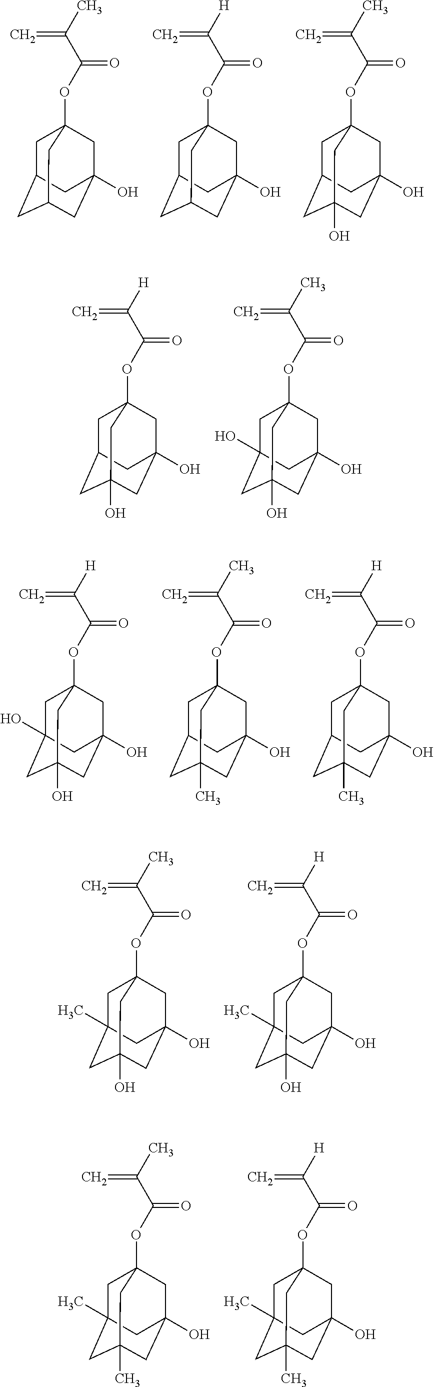

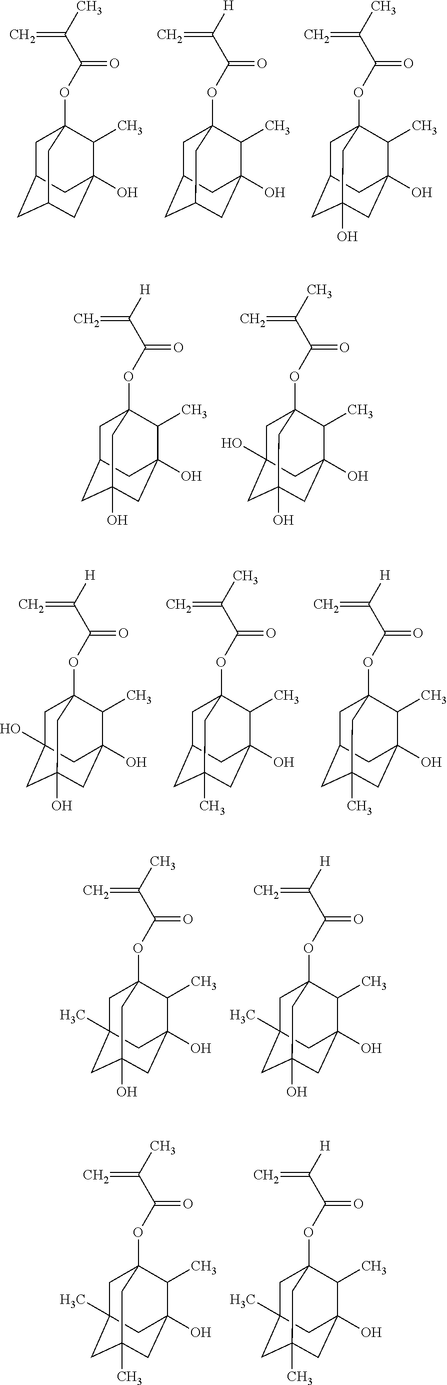

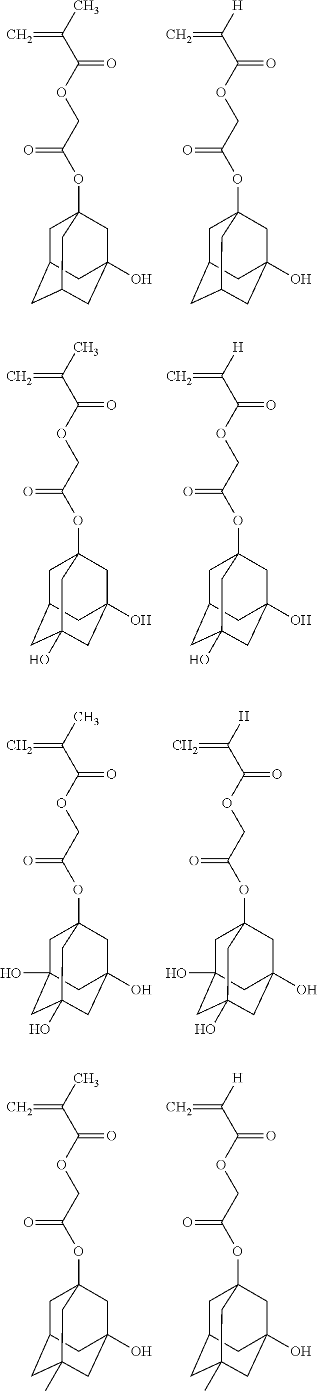

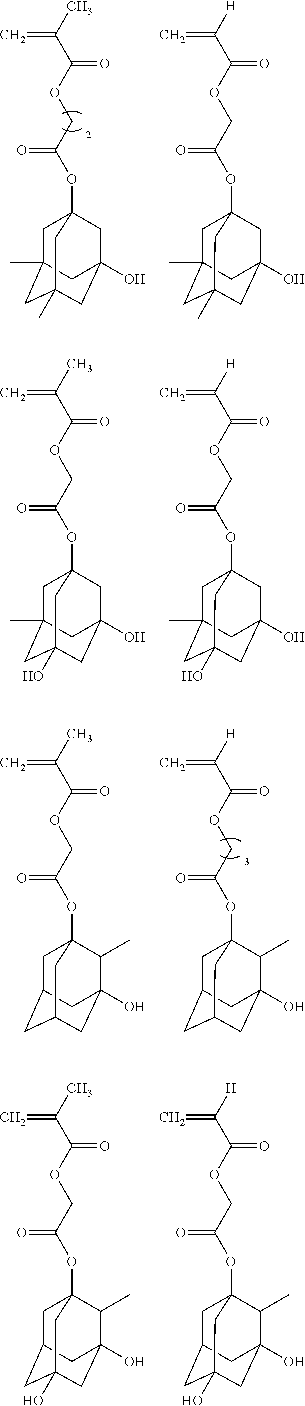

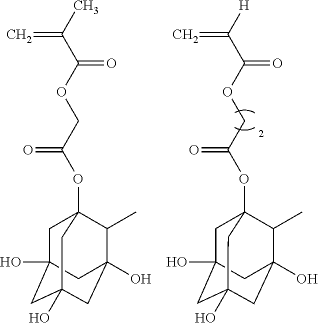

Among them, preferred are 3-hydroxy-1-adamantyl acrylate, 3-hydroxy-1-adamantyl methacrylate, 3,5-dihydroxy-1-adamantyl acrylate, 3,5-dihydroxy-1-adamantyl methacrylate, 1-(3,5-dihydroxy-1-adamantyloxycarbonyl)methyl acrylate and 1-(3,5-dihydroxy-1-adamantyloxycarbonyl)methylmethacrylate, and more preferred are 3-hydroxy-1-adamantyl methacrylate and 3,5-dihydroxy-1-adamantyl methacrylate.

When the resin of the present invention contains the structural unit derived from the monomer represented by the formula (a2-1), the content of the structural unit derived from the monomer represented by the formula (a2-1) is usually 3 to 40% by mole and preferably 5 to 35% by mole and more preferably 5 to 30% by mole based on total molar of all the structural units of the resin.

Examples of the lactone ring of the acid-stable monomer having a lactone ring and no acid-labile group include a monocyclic lactone ring such as β-propiolactone ring, γ-butyrolactone ring and γ-valerolactone ring, and a condensed ring formed from a monocyclic lactone ring and the other ring. Among them, preferred are γ-butyrolactone ring and a condensed lactone ring formed from γ-butyrolactone ring and the other ring.

Preferable examples of the acid-stable monomer having a lactone ring and no acid-labile group include the monomers represented by the formulae (a3-1), (a3-2) and (a3-3):

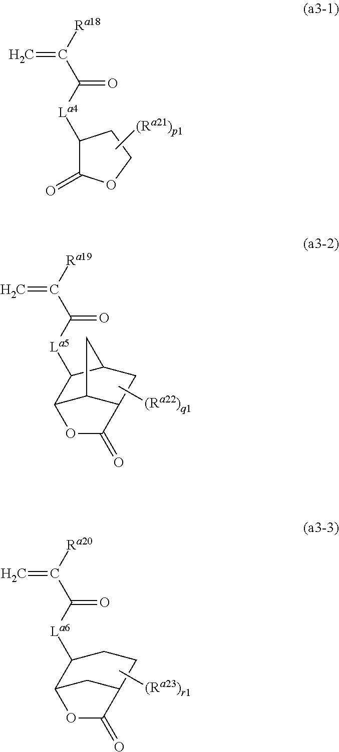

wherein L^{a4}, L^{a5} and L^{a6} independently represent *—O— or *—O— (CH₂)ₖ₃—CO—O— in which * represents a binding position to —CO— and k3 represents an integer of 1 to 7, R^{a18}, R^{a19} and R^{a20} independently represent a hydrogen atom or a methyl group, R^{a21} represents a C1-C4 aliphatic hydrocarbon group, R^{a22} and R^{a23} are independently in each occurrence a carboxyl group, a cyano group or a C1-C4 aliphatic hydrocarbon group, and p1 represents an integer of 0 to 5, q1 and r1 independently represent an integer of 0 to 3.

Examples of L^{a4}, L^{a5} and L^{a6} include the same as described in L^{a3}. It is preferred that L^{a4}, L^{a5} and L^{a6} each independently represent *—O— or *—O— (CH₂)_{d1}—CO—O— in which * represents a binding position to —CO— and d1 represents an integer of 1 to 4, and it is more preferred that L^{a4}, L^{a5} and L^{a6} are *—O—. R^{a18}, R^{a19} and R^{a20} are preferably methyl groups. R^{a21} is preferably a methyl group.

It is preferred that R^{a22} and R^{a23} are independently in each occurrence a carboxyl group, a cyano group or a methyl group. It is preferred that p1 is an integer of 0 to 2, and it is more preferred that p1 is 0 or 1. It is preferred that q1 and r1 independently each represent an integer of 0 to 2, and it is more preferred that q1 and r1 independently represent 0 or 1.

Examples of the monomer represented by the formula (a3-1) include the followings.

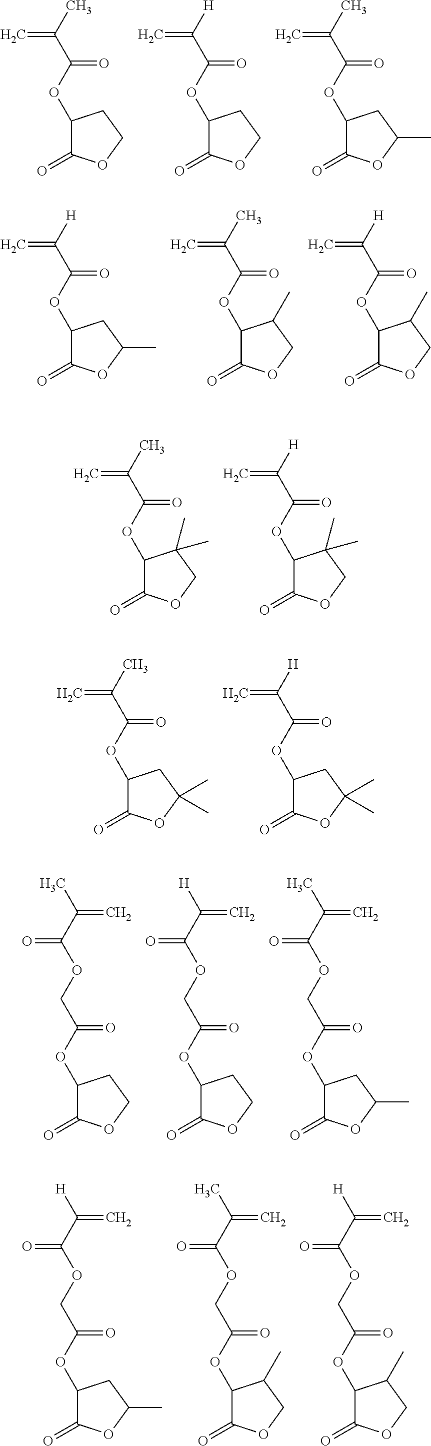

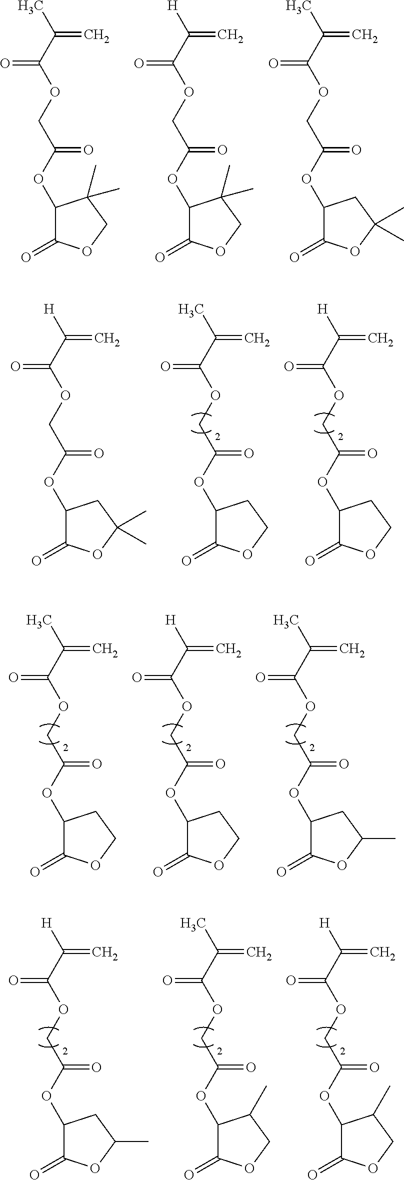

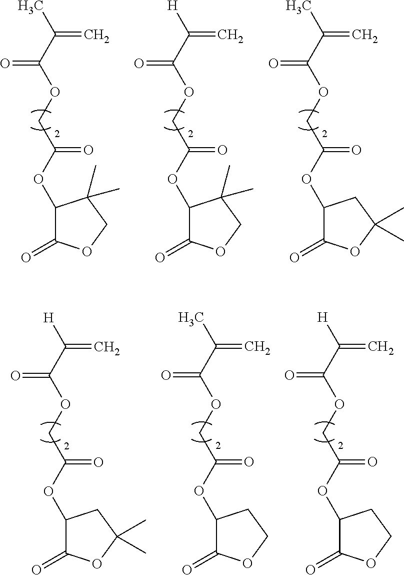

While the following monomer is an acid-labile monomer having a lactone ring, the resin can contain the structural unit derived from the following monomer.

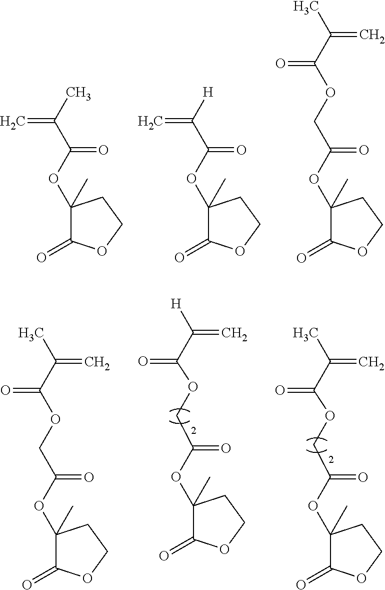

Examples of the monomer represented by the formula (a3-2) include the followings.

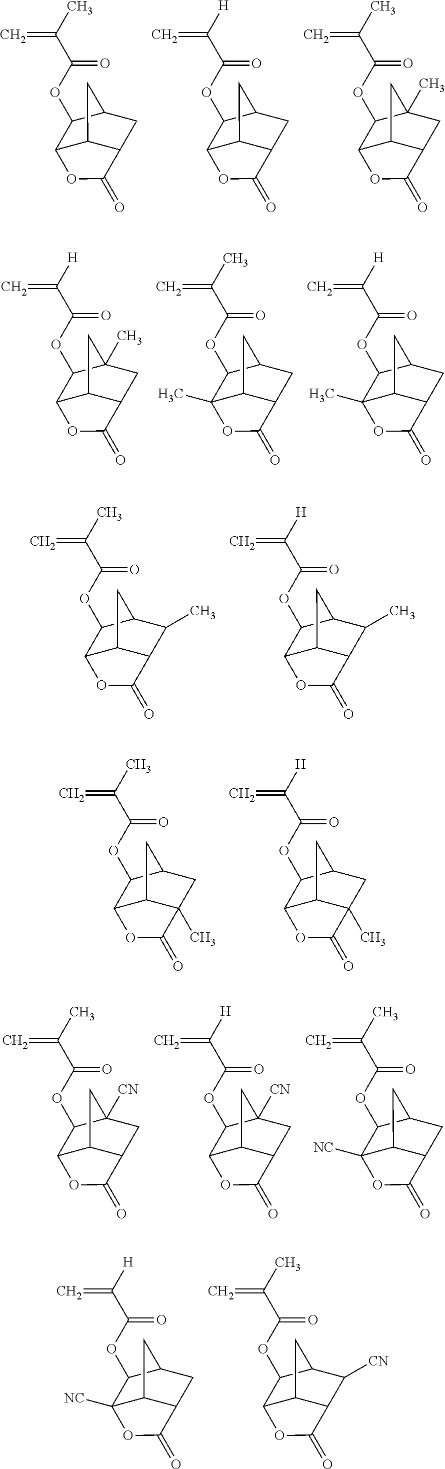

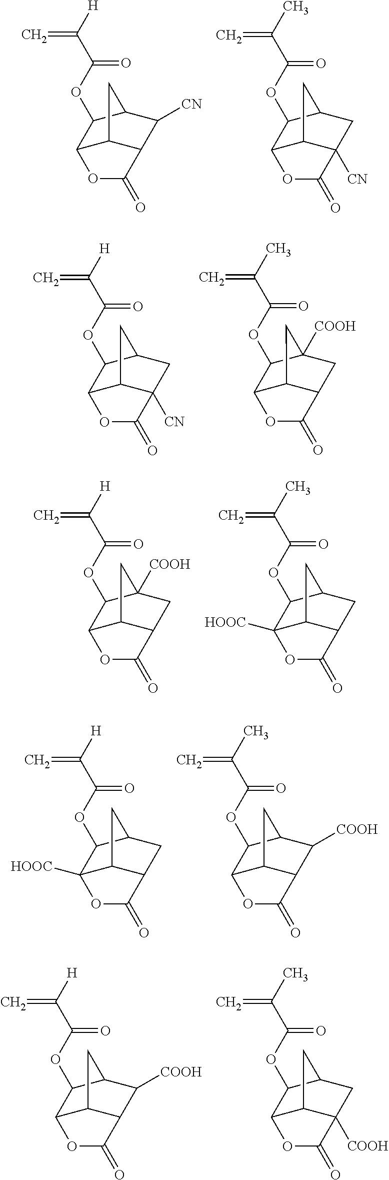

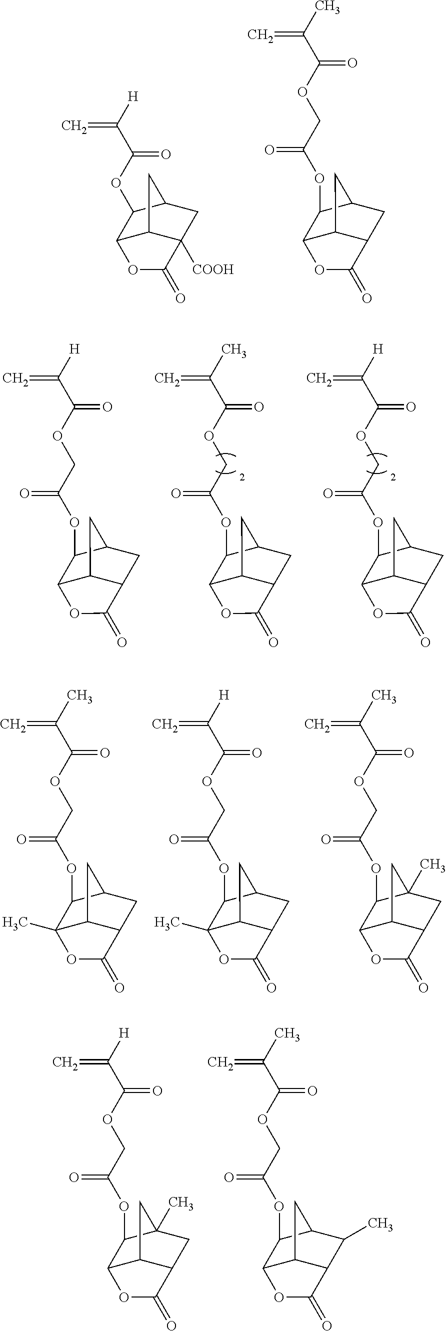

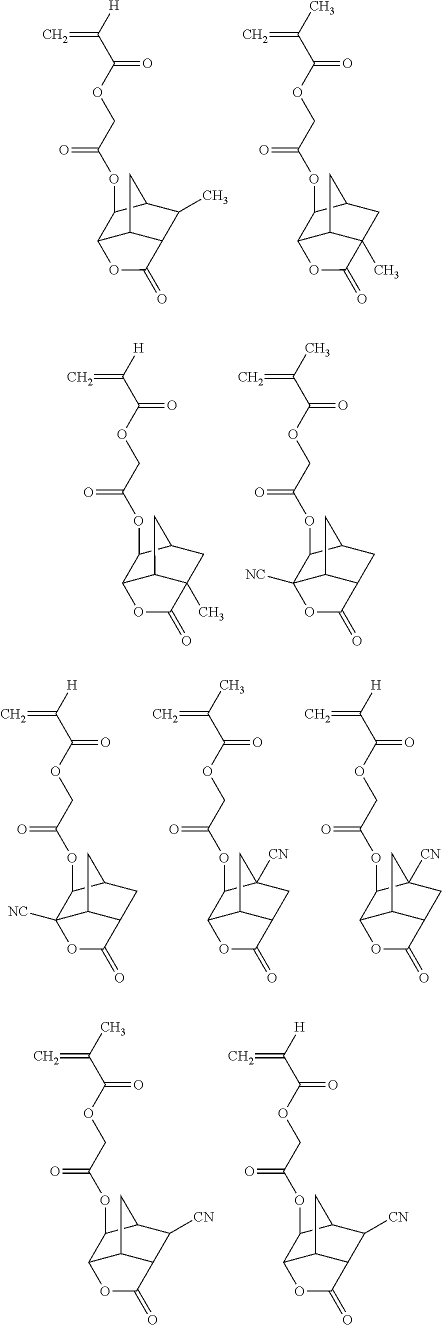

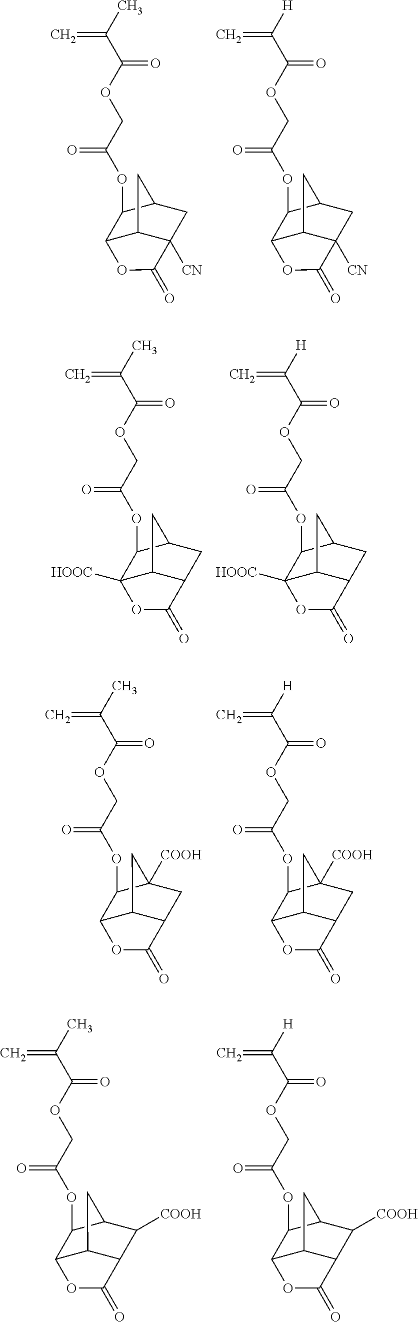

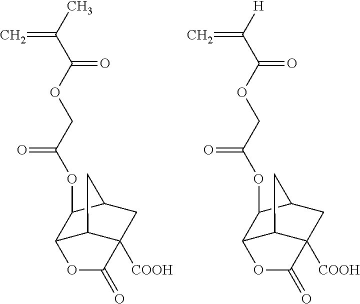

While the following monomer is an acid-labile monomer having a lactone ring, the resin can contain the structural unit derived from the following monomer.

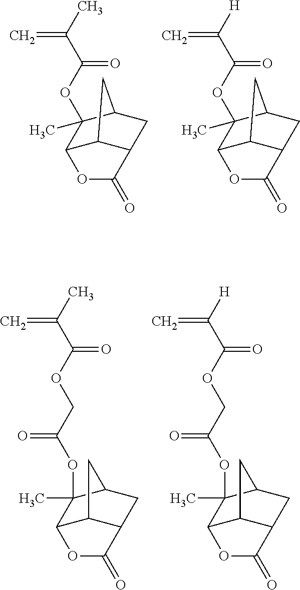

Examples of the monomer represented by the formula (a3-3) include the followings.

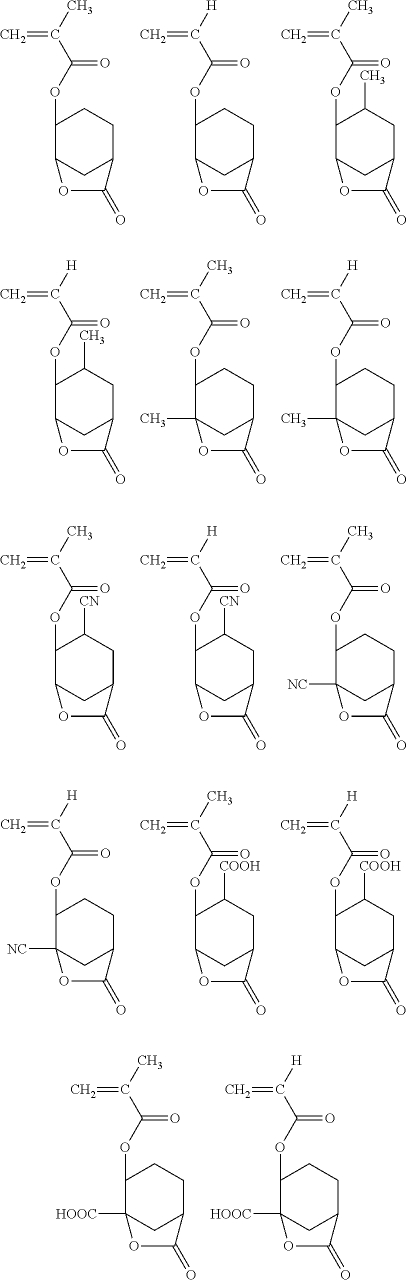

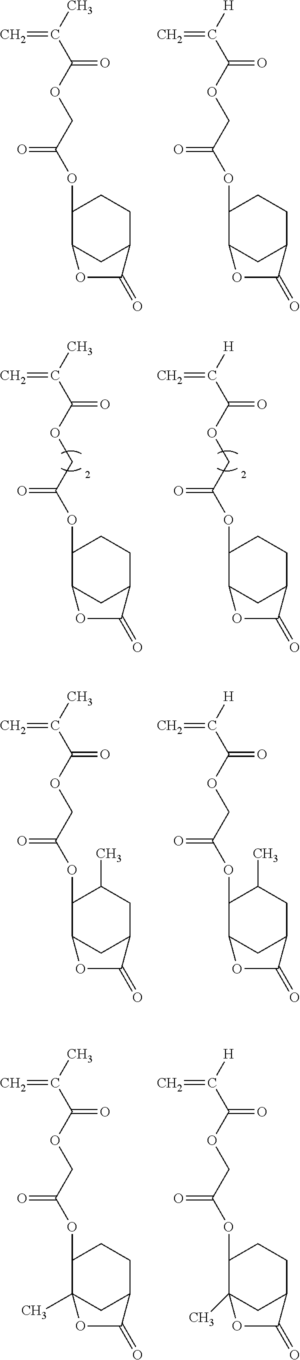

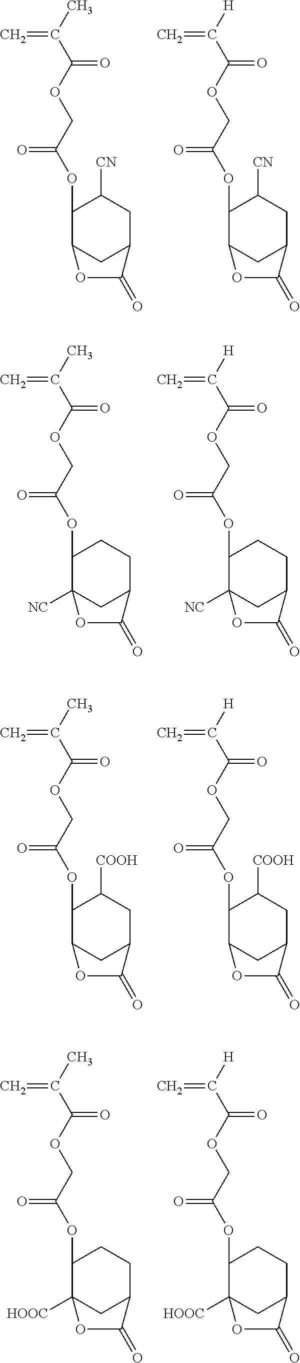

While the following monomer is an acid-labile monomer having a lactone ring, the resin can contain the structural unit derived from the following monomer.

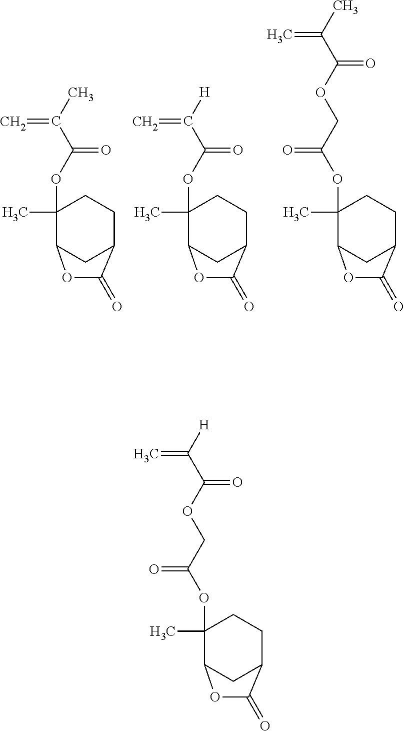

Among them, preferred are 5-oxo-4-oxatricyclo[4.2.1.0^{3,7}]nonan-2-yl acrylate, 5-oxo-4-oxatricyclo[4.2.1.0^{3,7}]nonan-2-yl methacrylate, tetrahydro-2-oxo-3-furyl acrylate, tetrahydro-2-oxo-3-furyl methacrylate, 2-(5-oxo-4-oxatricyclo[4.2.1.0^{3,7}]nonan-2-yloxy)-2-oxoethyl acrylate and 2-(5-oxo-4-oxatricyclo[4.2.1.0^{3,7}]nonan-2-yloxy)-2-oxoethyl methacrylate, and more preferred are 5-oxo-4-oxatricyclo[4.2.1.0^{3,7}]nonan-2-yl methacrylate, tetrahydro-2-oxo-3-furyl methacrylate and 2-(5-oxo-4-oxatricyclo[4.2.1.0^{3,7}]nonan-2-yloxy)-2-oxoethyl methacrylate.

When the resin of the present invention contains the structural unit derived from the acid-stable monomer having a lactone ring and no acid-labile group, the content thereof is usually 5 to 50% by mole and preferably 10 to 45% by mole and more preferably 15 to 40% by mole based on total molar of all the structural units of the resin.

The resin of the present invention can be produced according to known polymerization methods such as radical polymerization.

The resin of the present invention preferably has 2,500 or more of the weight-average molecular weight, and preferably 3,000 or more of the weight-average molecular weigh. The resin of the present invention preferably has 50,000 or less of the weight-average molecular weight, and preferably 30,000 or less of the weight-average molecular weight. The weight-average molecular weight can be measured with gel permeation chromatography.

The resin of the present invention is preferably a copolymer of the compound (I), the monomer having an acid-labile group different from the compound (I) and the acid-stable monomer having no acid-labile group, and more preferably a copolymer comprising the structural unit derived from the compound (I), the structural unit derived from the monomer having an acid-labile group different from the compound (I) and at least one selected from the group consisting of the structural unit derived from the acid-stable monomer having no acid-labile group and having one or more hydroxyl groups and the structural unit derived from the acid-stable monomer having no acid-labile group and having a lactone ring. The monomer having an acid-labile group different from the compound (I) is preferably the monomer (a1-1) or (a1-2), and more preferably the monomer (a1-1). The acid-stable monomer having no acid-labile group and having one or more hydroxyl groups is preferably the monomer (a2-0), (a2-10) or (a2-1), and more preferably the monomer (a2-10) or (a2-1). The acid-stable monomer having no acid-labile group and having a lactone ring is preferably the monomer (a3-1) or (a3-2).

The photoresist composition of the present invention contains the resin of the present invention. The content of the resin in the photoresist composition is usually 70 to 99.9% by weight and preferably 80 to 99.9% by weight based on sum of solid component. The photoresist composition of the present invention usually contains an acid generator. The content of the acid generator is usually 0.1 to 30% by weight and preferably 0.1 to 20% by weight based on sum of solid component. Herein, “solid component” means the components other than a solvent among all components of the photoresist composition.

The acid generator is a substance which is decomposed to generate an acid by applying a radiation such as a light, an electron beam or the like on the substance itself or on a photoresist composition containing the substance. The acid generated from the acid generator acts on the resin resulting in cleavage of the acid-labile group existing in the resin.

Examples of the acid generator include a nonionic acid generator, an ionic acid generator and the combination thereof. Examples of the nonionic acid generator include an organo-halogen compound, a sulfone compound such as a disulfone, a ketosulfone and a sulfonyldiazomethane, a sulfonate compound such as a 2-nitrobenzylsulfonate, an aromatic sulfonate, an oxime sulfonate, an N-sulfonyloxyimide, a sulfonyloxyketone and DNQ 4-sulfonate.

Examples of the ionic acid generator include an onium salt compound such as a diazonium salt, a phosphonium salt, a sulfonium salt and an iodonium salt. Examples of the anion of the onium salt include a sulfonic acid anion, a sulfonylimide anion and a sulfonulmethide anion. The onium salt compound is preferable.

Other examples of the acid generator include acid generators described in JP 63-26653 A, JP 55-164824 A, JP 62-69263 A, JP 63-146038 A, JP 63-163452 A, JP 62-153853 A, JP 63-146029 A, U.S. Pat. No. 3,779,778, U.S. Pat. No. 3,849,137, DE Patent No. 3914407 and EP Patent No. 126,712.

Preferable examples of the acid generator include a fluorine-containing acid generator, and more preferable acid generator is a salt represented by the formula (B1):

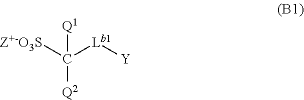

wherein Q¹ and Q² each independently represent a fluorine atom or a C1-C6 perfluoroalkyl group,
L^{b1} represents a single bond or a C1-C17 saturated divalent hydrocarbon group which can have one or more substituents, and one or more —CH₂— in the saturated divalent hydrocarbon group can be replaced by —O— or —CO—,
Y represents a C1-C18 aliphatic hydrocarbon group or a C3-C18 saturated cyclic hydrocarbon group, and the aliphatic hydrocarbon group and the saturated cyclic hydrocarbon group can have one or more substituents, and one or more —CH₂— in the aliphatic hydrocarbon group and the saturated cyclic hydrocarbon group can be replaced by —O—, —SO₂— or —CO—,
Z⁺ represents an organic cation.

Examples of the C1-C6 perfluoroalkyl group include a trifluoromethyl group, a pentafluoroethyl group, a heptafluoropropyl group, a nonafluorobutyl group, an undecafluoropentyl group and a tridecafluorohexyl group, and a trifluoromethyl group is preferable. Q¹ and Q² each independently preferably represent a fluorine atom or a trifluoromethyl group, and Q¹ and Q² are more preferably fluorine atoms.

Examples of the C1-C17 saturated divalent hydrocarbon group include a C1-C17 alkylene group and a divalent group having an alicyclic divalent hydrocarbon group. Examples of the alkylene group include a linear alkanediyl group such as a methylene group, an ethylene group, a propane-1,3-diyl group, a butane-1,4-diyl group, a pentane-1,5-diyl, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group and a heptadecane-1,17-diyl group, a branched chain alkanediyl group formed by replacing one or more hydrogen atom of the above-mentioned linear alkanediyl group by a C1-C4 alkyl group such as a 1-methylpropane-1,3-diyl group, a 2-methylpropane-1,3-diyl group, a 2-methylpropane-1,2-diyl group, a 1-methylbutane-1,4-diyl group and a 2-methylbutane-1,4-diyl group,

a divalent saturated monocyclic hydrocarbon group such as a cycloalkylene group such as a 1,3-cyclobutylene group, a 1,3-cyclopentylene group, a 1,4-cyclohexylene group and 1,5-cyclooctylene group,
a divalent saturated polycyclic hydrocarbon group such as a norbornane-1,4-diyl group, a norbornane-2,5-diyl group, a adamantane-1,5-diyl group and a adamantane-2,6-diyl group, and a group formed by combining two or more groups selected from the group consisting of the above-mentioned groups.

Examples of the C1-C17 divalent saturated hydrocarbon group in which one or more —CH₂— are replaced by —O— or —CO— include *—CO—O-L^{b2}, *—CO—O-L^{b4}-CO—O-L^{b3}-, *-L^{b5}-O—CO—, *-L^{b7}-O-L^{b6}-, *—CO—O-L^{b8}-O—, and *—CO—O-L^{b10}-O-L^{b9}-CO—O—, wherein L^{b2} represents a single bond or a C1-C15 saturated hydrocarbon group, L^{b3} represents a single bond or a C1-C12 saturated hydrocarbon group, L^{b4} represents C1-C13 saturated hydrocarbon group, with the proviso that total carbon number of L^{b3} and L^{b4} is 1 to 13, L^{b5} represents a C1-C15 saturated hydrocarbon group, L^{b6} represents a C1-C15 saturated hydrocarbon group, L^{b7} represents a C1-C15 saturated hydrocarbon group, with the proviso that total carbon number of L^{b6} and L^{b7} is 1 to 16, L^{b8} represents a C1-C14 saturated hydrocarbon group, L^{b9} represents a C1-C11 saturated hydrocarbon group, L^{b10} represents a C1-C11 saturated hydrocarbon group, with the proviso that total carbon number of L^{b9} and L^{b10} is 1 to 12, and * represents a binding position to —C(R¹) (R²)—. Among them, preferred is *—CO—O-L^{b2}-, and more preferred is *—CO—O-L^{b2}- in which L^{b2} is a single bond or —CH₂—.

Examples of *—CO—O-L^{b2}- include *—CO—O— and *—CO—O—CH₂—. Examples of *—CO—O-L^{b4}-CO—O-L^{b3}- include *—CO—O—CH₂—CO—O—, *—CO—O—(CH₂)₂—CO—O—, *—CO—O—(CH₂)₃—CO—O—, *—CO—O—(CH₂)₄—CO—O—, *—CO—O—(CH₂)₆—CO—O—, *—CO—O—(CH₂)₈—CO—O—, *—CO—O—CH₂—CH(CH₃)—CO—O— and *—CO—O—CH₂—C(CH₃)₂—CO—O—. Examples of *-L^{b5}-O—CO— include *—CH₂—O—CO—, *—(CH₂)₂—O—CO—, *—(CH₂)₃—O—CO—, *—(CH₂)₄—O—CO—, *—(CH₂)₆—O—CO— and *—(CH₂)₈—O—CO—. Examples of *-L^{b6}-O-L^{b6}- include *—CH₂—O—CH₂—. Examples of *—CO—O-L^{b8}-O— include *—CO—O—CH₂—O—, *—CO—O—(CH₂)₂—O—, *—CO—O—(CH₂)₃—O—, *—CO—O—(CH₂)₄—O— and *—CO—O—(CH₂)₆—O—. Examples of *—CO—O-L^{b10}-O-L^{b9}-CO—O— include the followings.

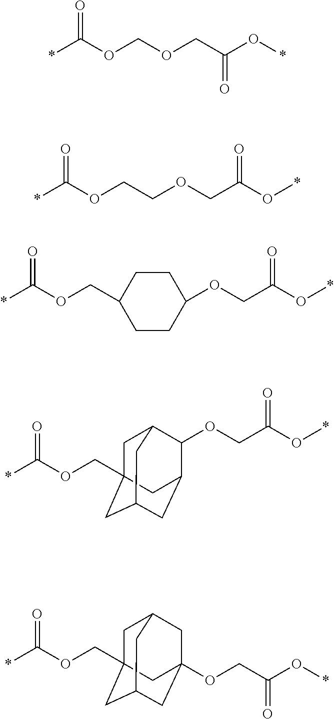

The saturated hydrocarbon group of L^{b1} may have one or more substituents, and examples thereof include a halogen atom, a hydroxyl group, a carboxyl group, a C6-C18 aromatic hydrocarbon group, a C7-C21 aralkyl group, a C2-C4 acyl group and a glycidyloxy group.

Examples of the aralkyl group include a benzyl group, a phenylethyl group, a phenylpropyl group, a trityl group, a naphthylmethyl group and a naphthylethyl group.

Examples of the substituent in Y include a halogen atom, a hydroxyl group, an oxo group, a glycidyloxy group, a C2-C4 acyl group, a C1-C12 alkoxy group, a C2-C7 alkoxycarbonyl group, a C1-C12 aliphatic hydrocarbon group, a C1-C12 hydroxy-containing aliphatic hydrocarbon group, a C3-C16 saturated cyclic hydrocarbon group, a C6-C18 aromatic hydrocarbon group, a C7-C21 aralkyl group and —(CH₂)ⱼ₂—O—CO—R^{b1}— in which R^{b1} represents a C1-C16 aliphatic hydrocarbon group, a C3-C16 saturated cyclic hydrocarbon group or a C6-C18 aromatic hydrocarbon group and j2 represents an integer of 0 to 4. Examples of the halogen atom include a fluorine atom, a chlorine atom, a bromine atom and an iodine atom. Examples of the acyl group include an acetyl group and a propionyl group, and examples of the alkoxy group include a methoxy group, an ethoxy group, a propoxy group, an isopropoxy group and a butoxy group. Examples of the alkoxycarbonyl group include a methoxycarbonyl group, an ethoxycarbonyl group, a propoxycarbonyl group, an isopropoxycarbonyl group and a butoxycarbonyl group. Examples of the aliphatic hydrocarbon group include the same as described above.

Examples of the hydroxyl-containing aliphatic hydrocarbon group include a hydroxymethyl group. Examples of the C3-C16 saturated cyclic hydrocarbon group include the same as described above, and examples of the aromatic hydrocarbon group include a phenyl group, a naphthyl group, an anthryl group, a p-methylphenyl group, a p-tert-butylphenyl group and a p-adamantylphenyl group. Examples of the aralkyl group include a benzyl group, a phenethyl group, a phenylpropyl group, a trityl group, a naphthylmethyl group and a naphthylethyl group.

Examples of the C1-C18 aliphatic hydrocarbon group represented by Y include a methyl group, an ethyl group, a propyl group, an isopropyl group, a butyl group, an isobutyl group, a sec-butyl group, a tert-butyl group, a pentyl group, a neopentyl group, a 1-methylbutyl group, a 2-methylbutyl group, a 1,2-dimethylpropyl group, a 1-ethylpropyl group, a hexyl group, a 1-methylpentyl group, a heptyl group, an octyl group, a 2-ethylhexyl group, a nonyl group, a decyl group, an undecyl group and a dodecyl group, and a C1-C6 alkyl group is preferable. Examples of the C3-C36 saturated cyclic hydrocarbon group represented by Y include the groups represented by the formulae (Y1) to (Y26):

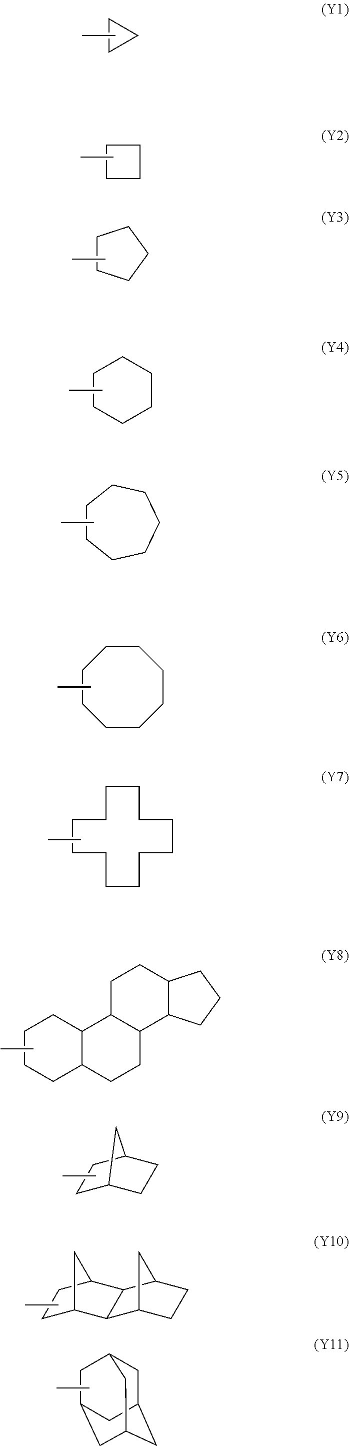

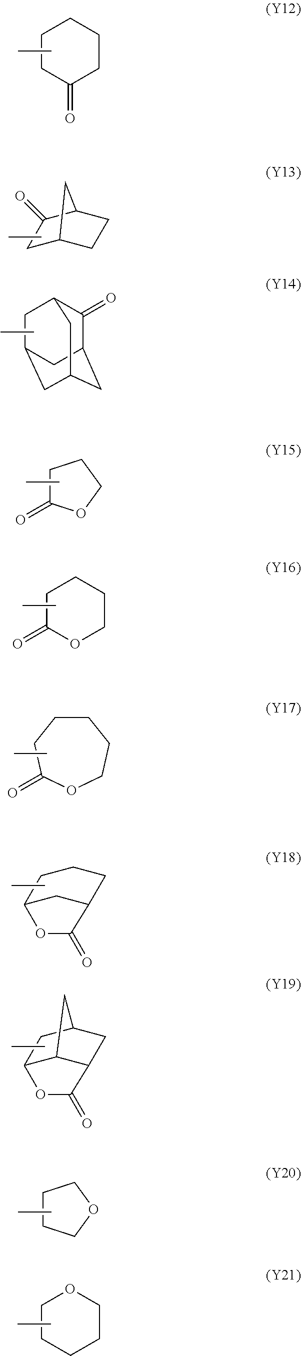

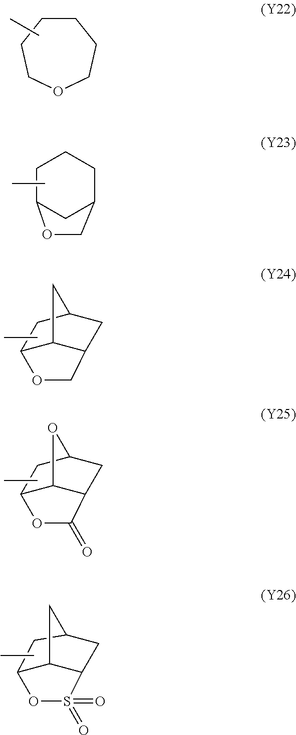

Among them, preferred are the groups represented by the formulae (Y1) to (Y19), and more preferred are the groups represented by the formulae (Y11), (Y14), (Y15) and (Y19). The groups represented by the formulae (Y11) and (Y14) are especially preferable.

Examples of Y having one or more substituents include the followings:

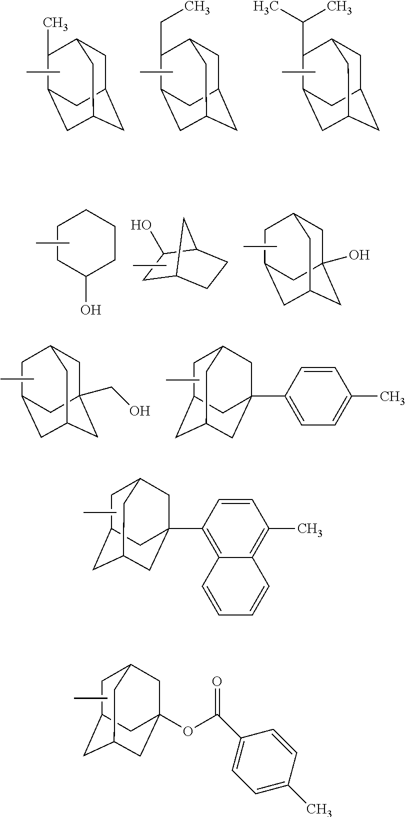

Y is preferably an adamantyl group which can have one or more substituents, and is more preferably an adamantyl group or an oxoadamantyl group.

Among the sulfonic acid anions of the salt represented by the formula (B1), preferred is a sulfonic acid anion in which L^{b1} is *—CO—O-L^{b2}-, and more preferred are anions represented by the formulae (b1-1-1) to (b1-1-9).

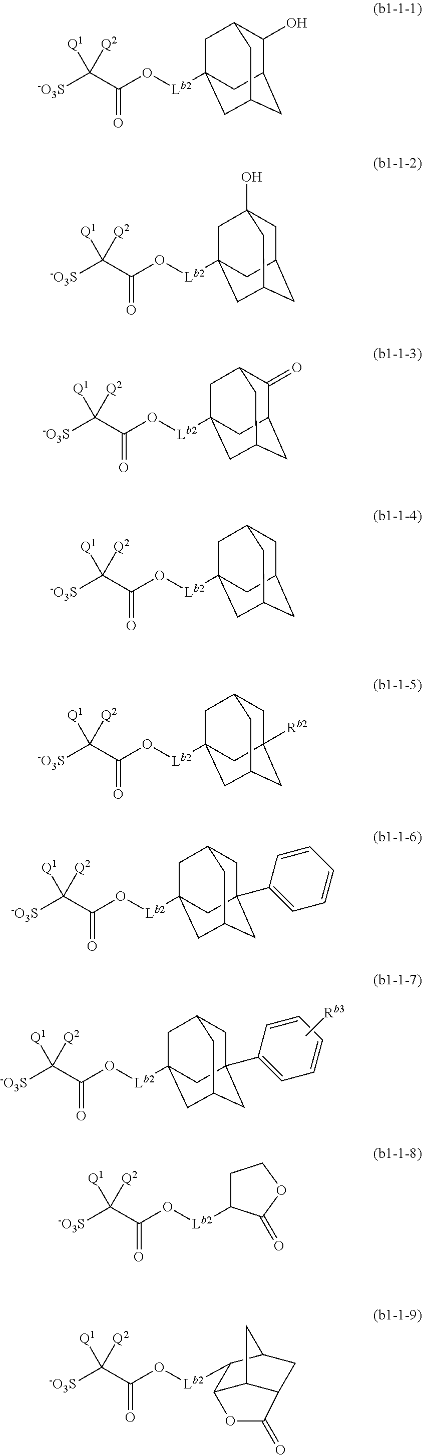

wherein Q¹, Q² and L^{b2} are the same as defined above, and R^{b2} and R^{b3} each independently represent a C1-C4 aliphatic hydrocarbon group, preferably a methyl group.

Examples of the anions of the salt represented by the formula (B1) include the following.

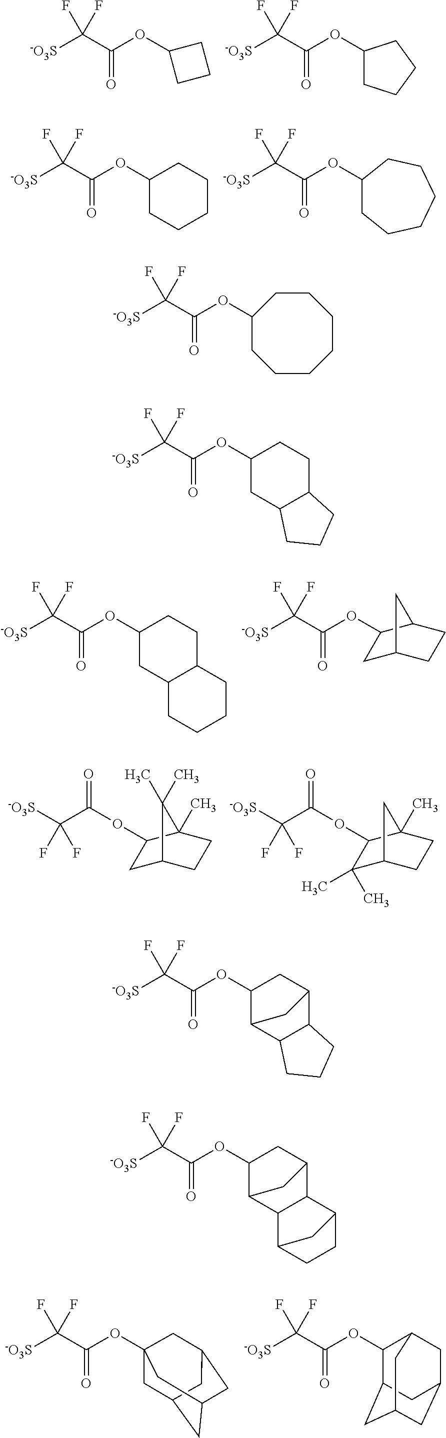

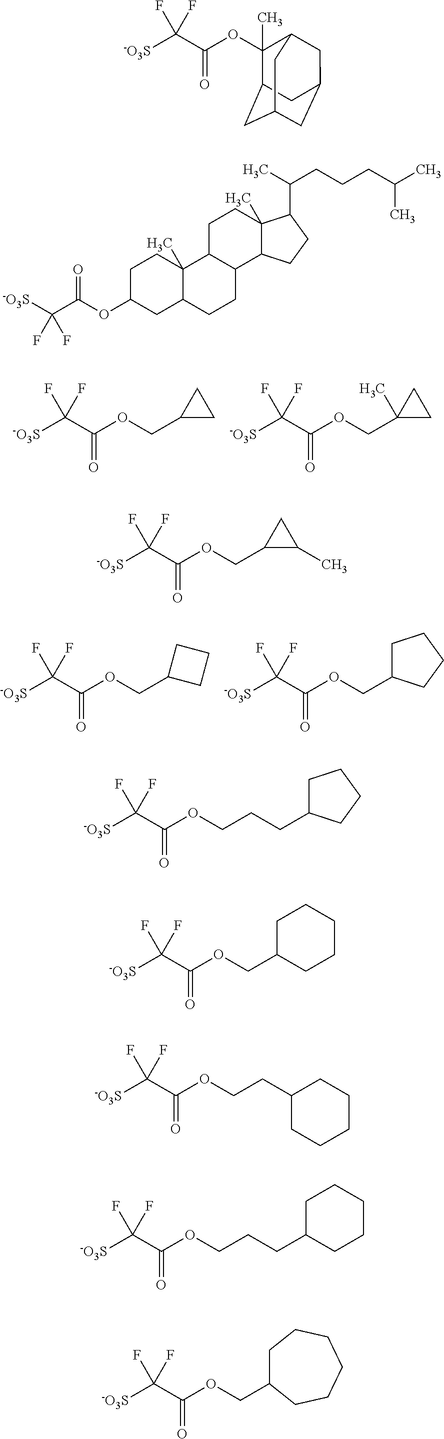

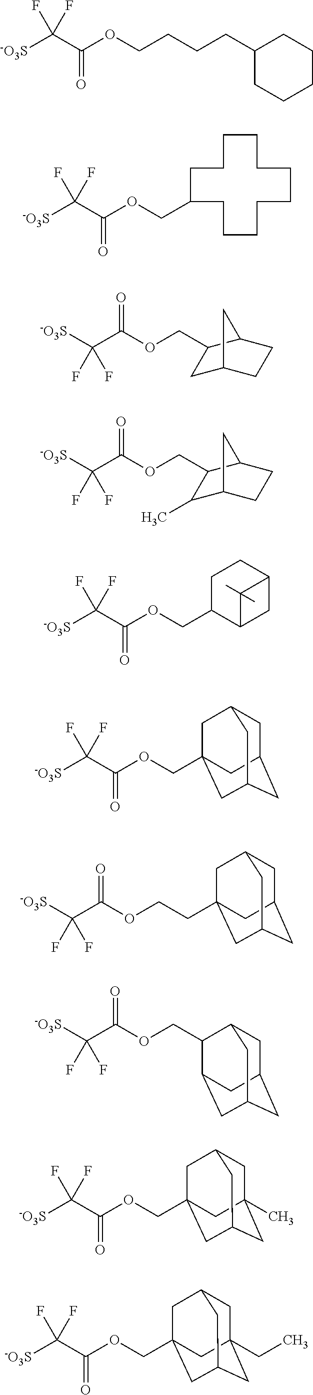

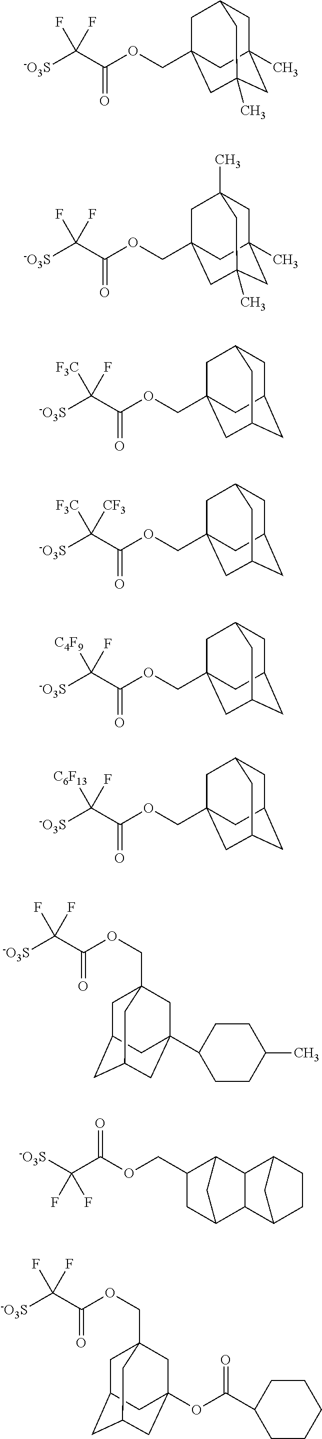

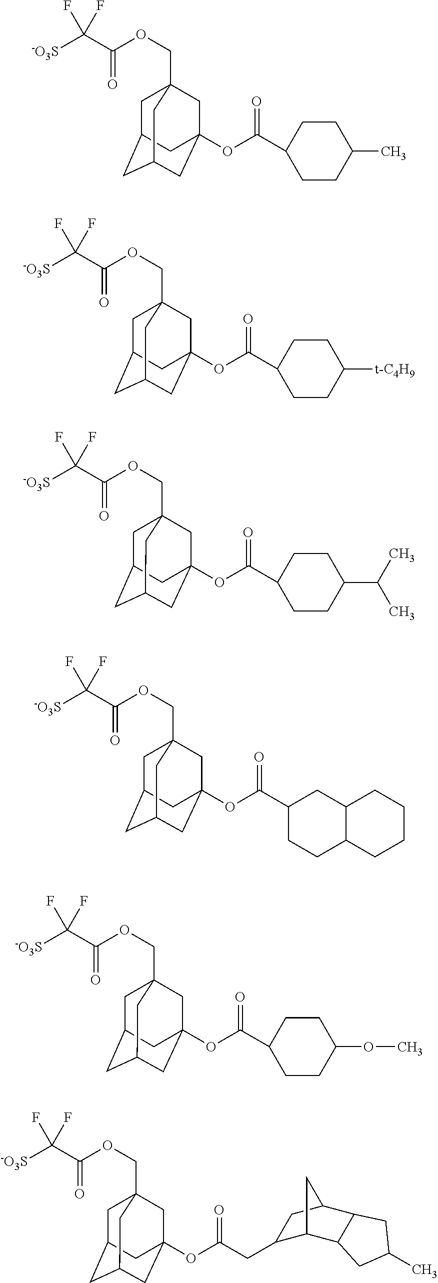

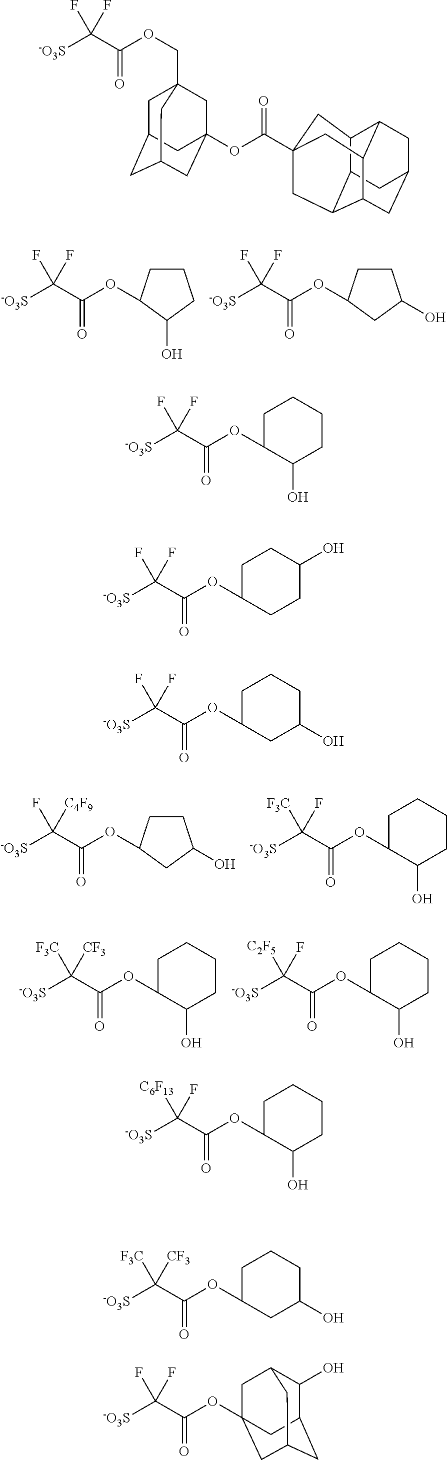

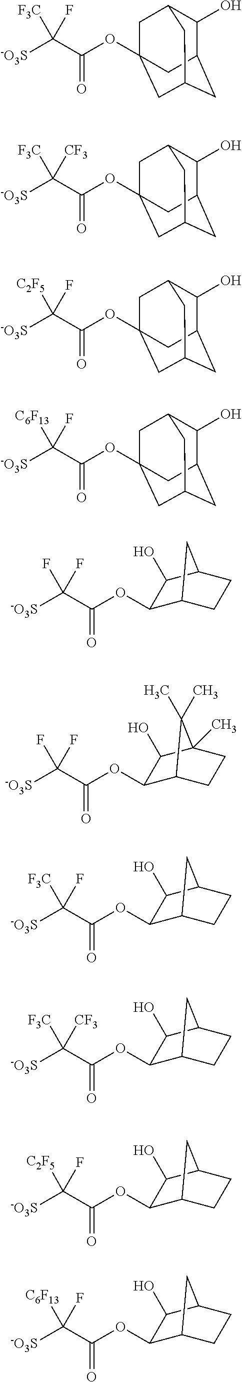

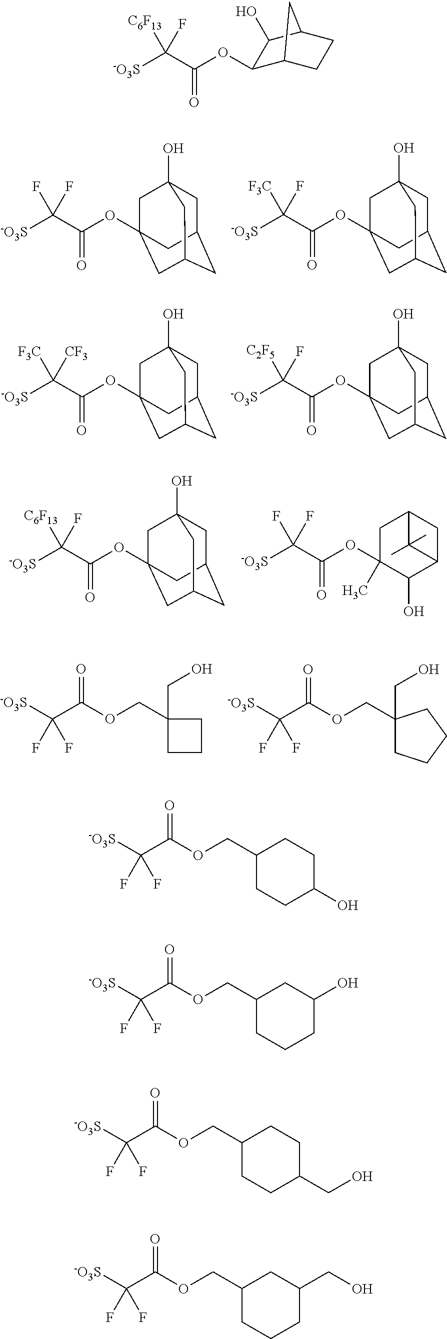

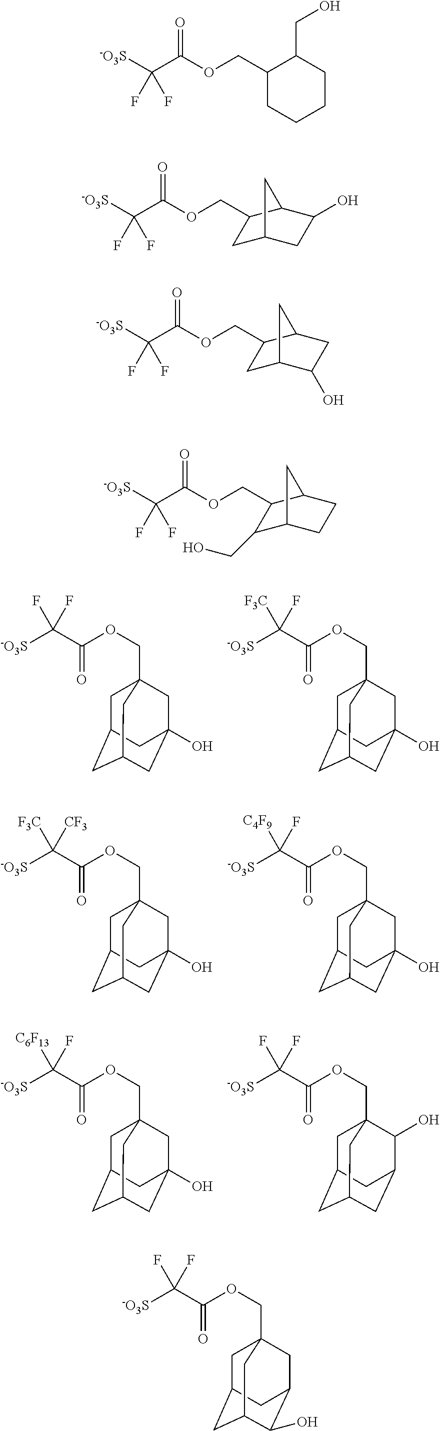

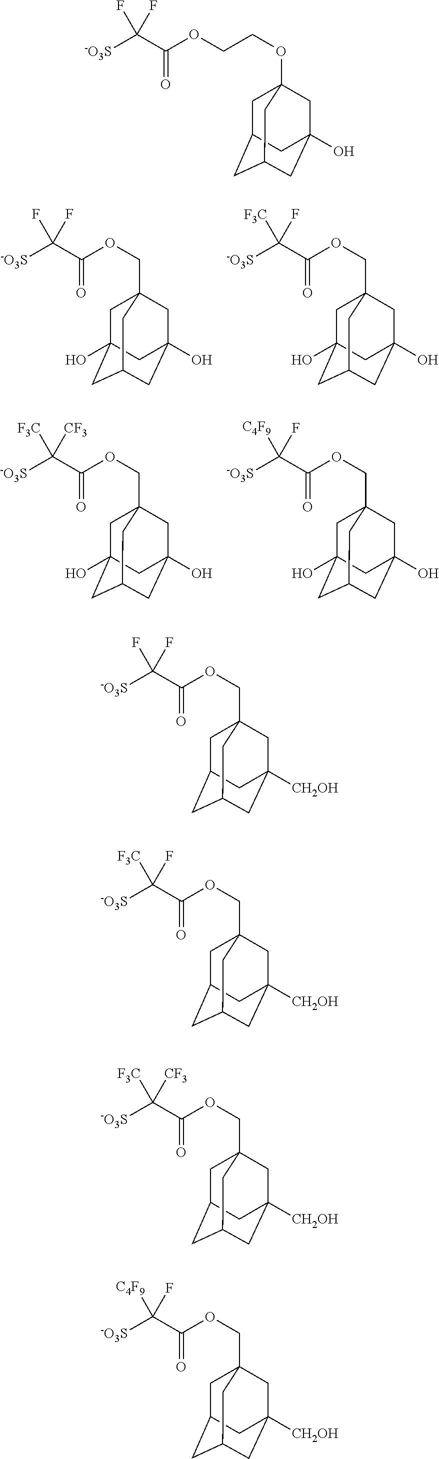

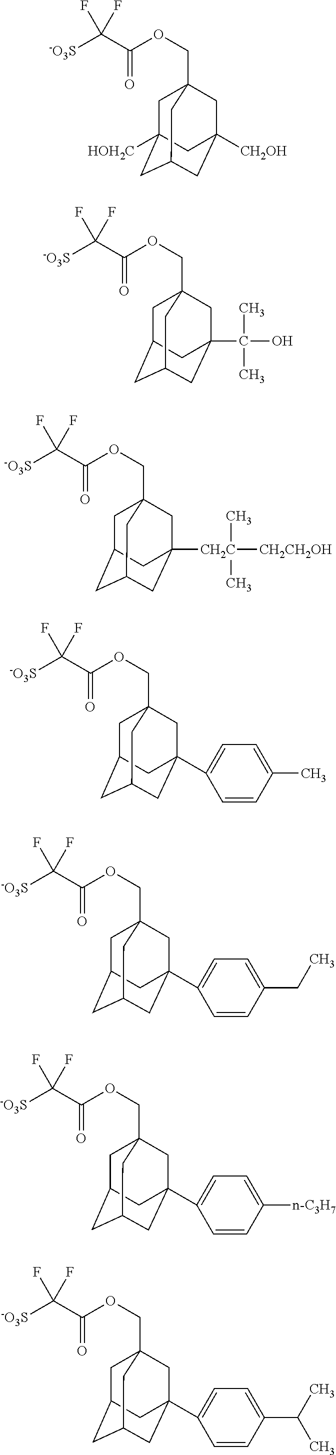

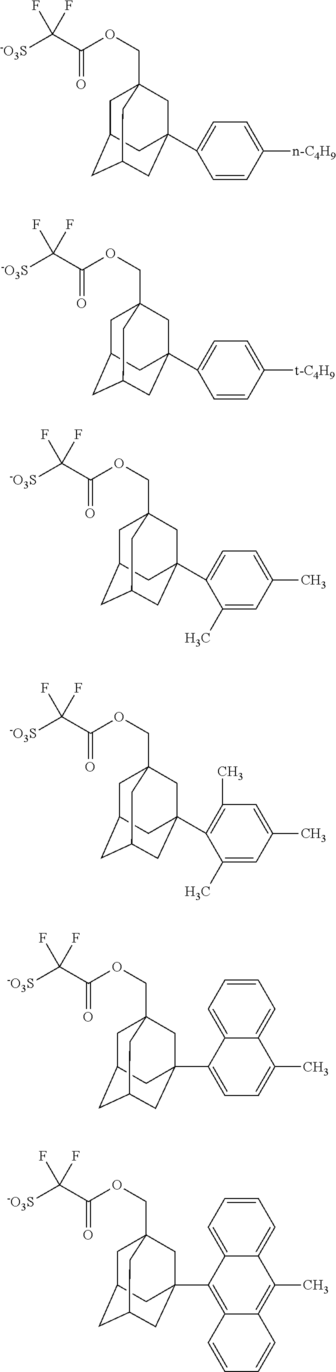

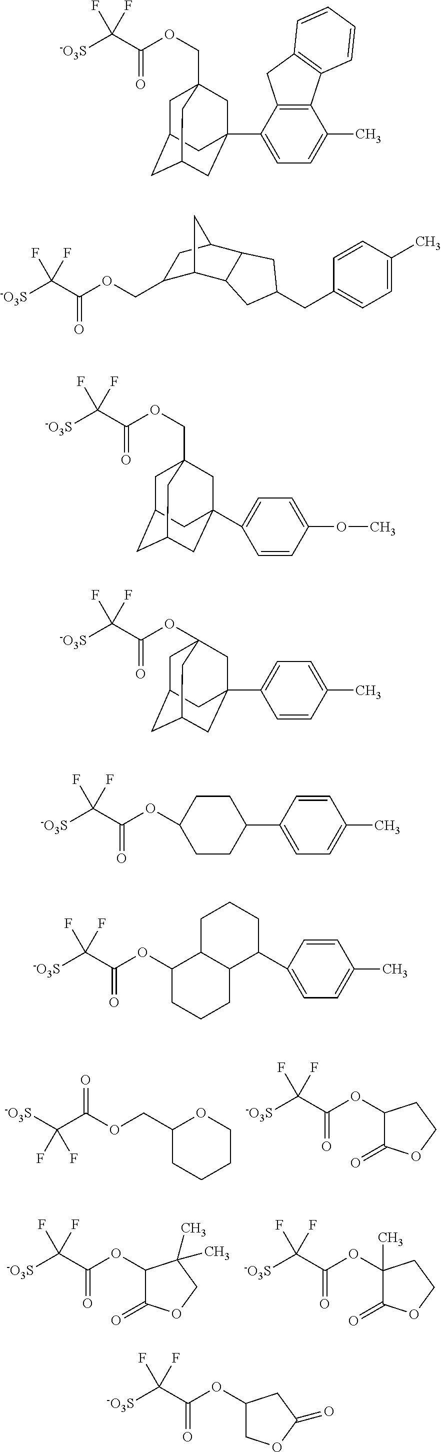

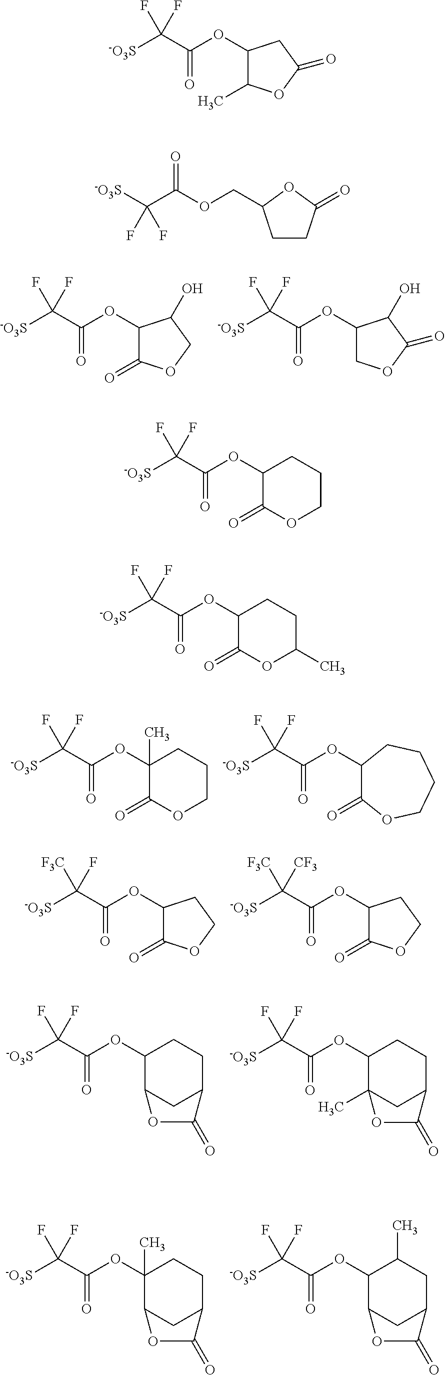

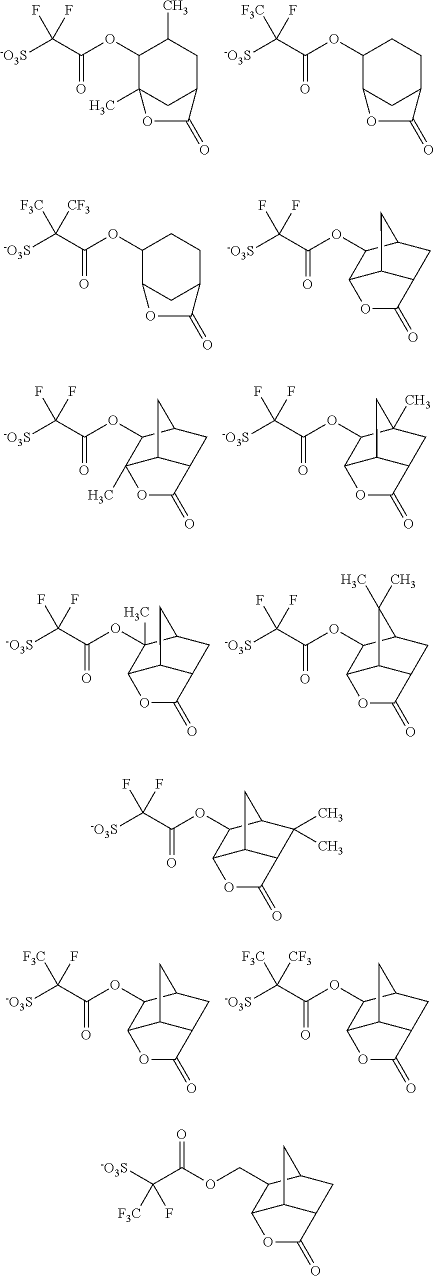

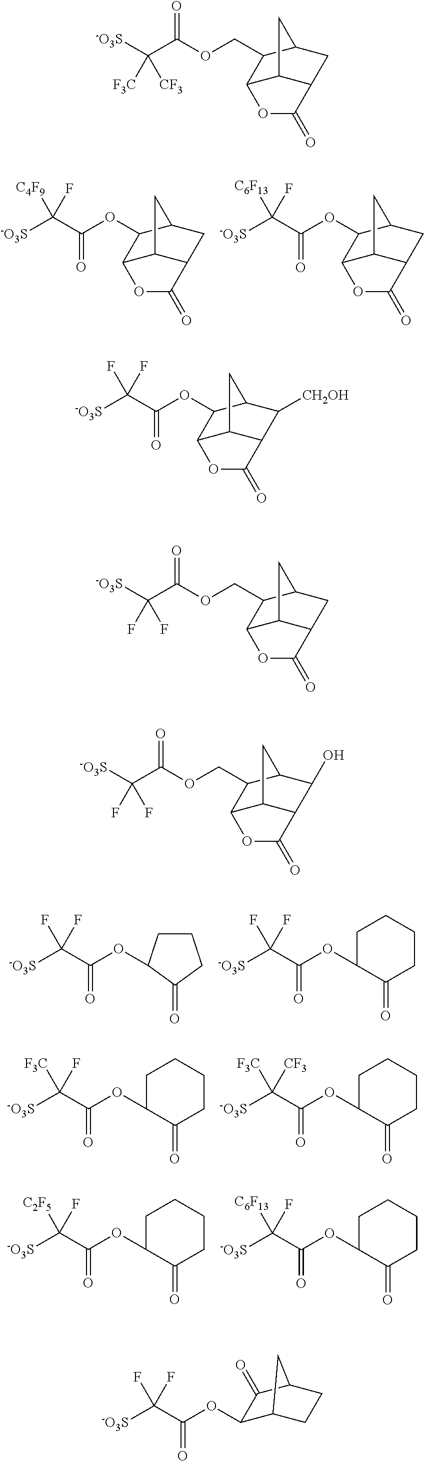

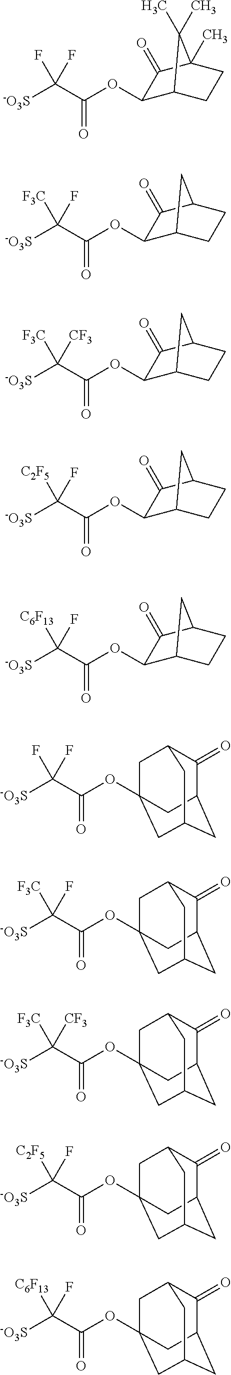

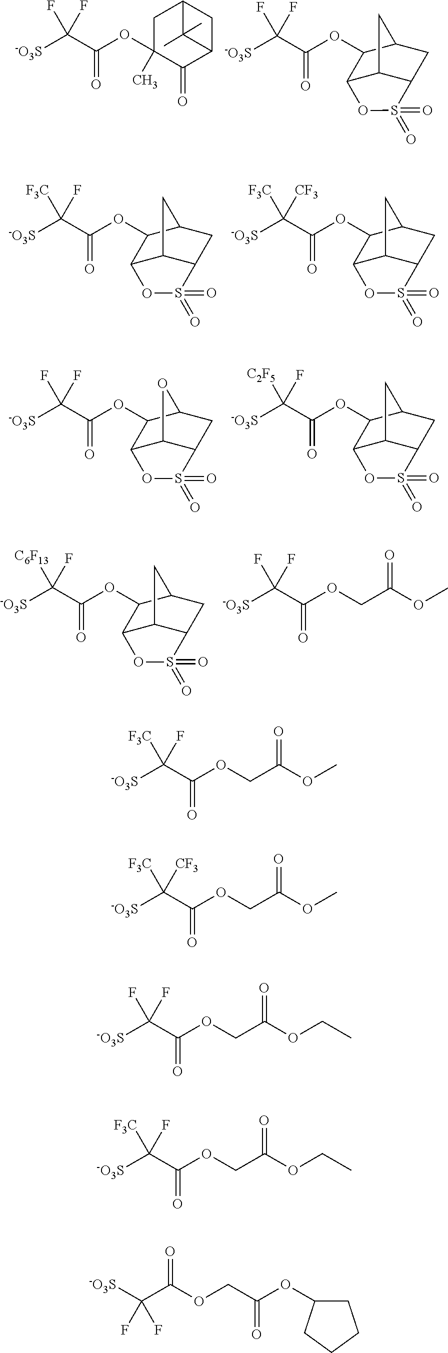

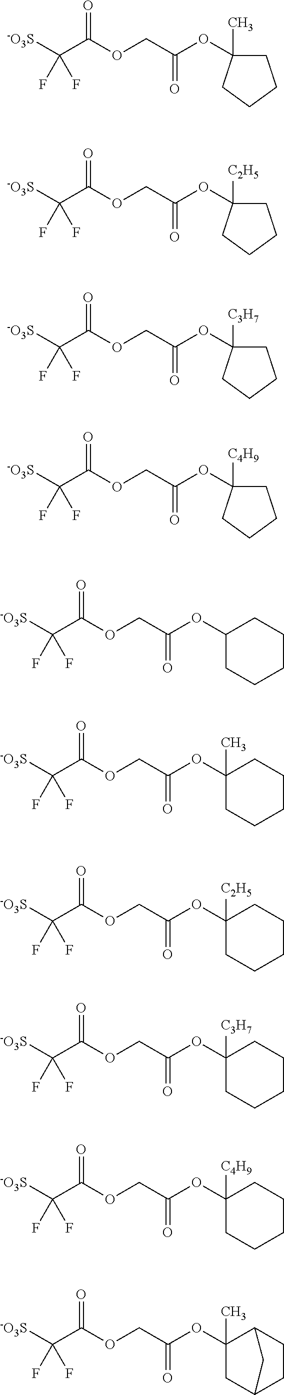

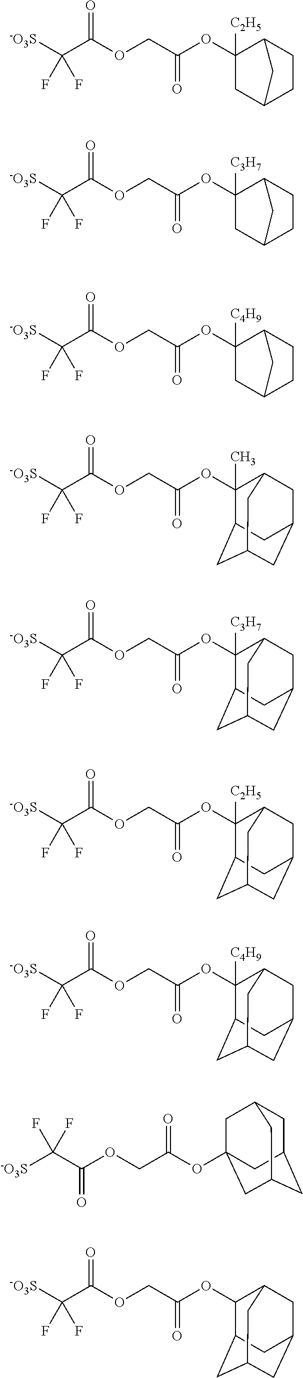

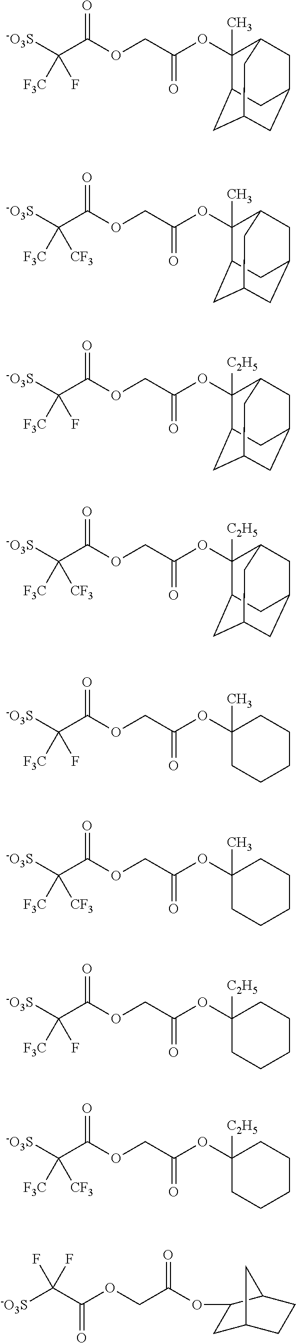

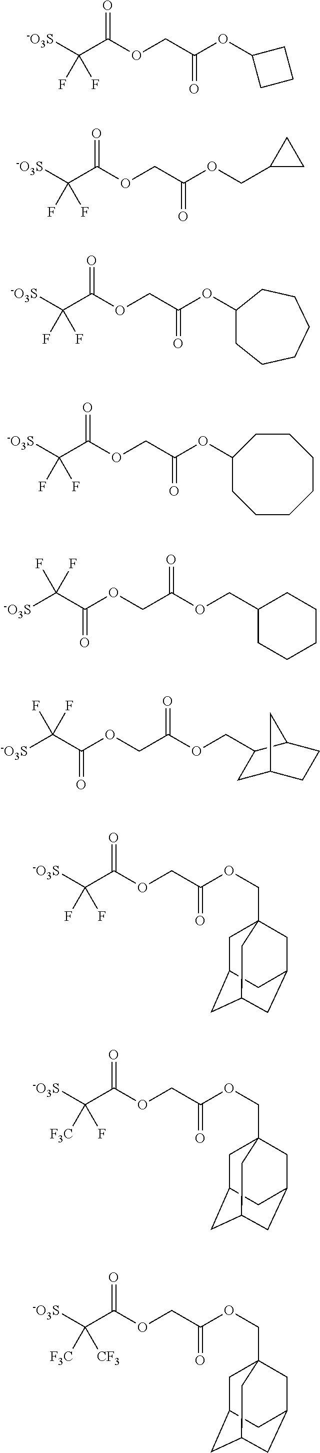

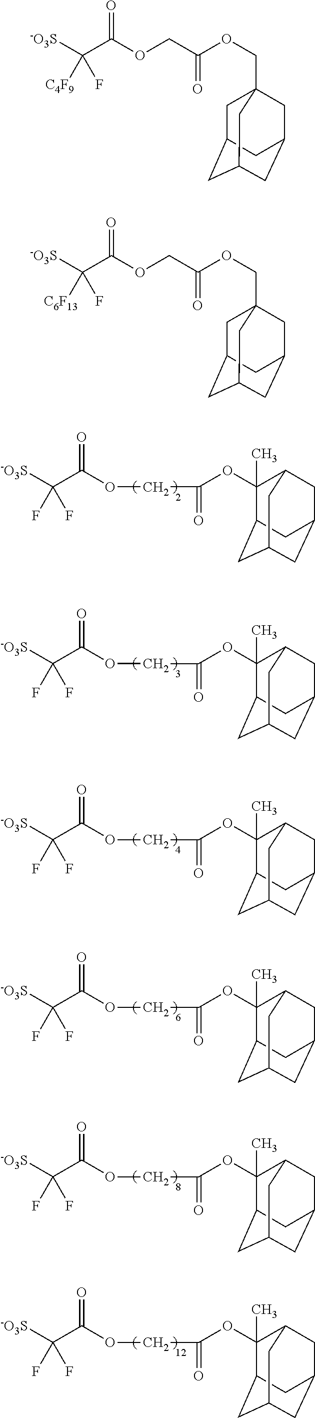

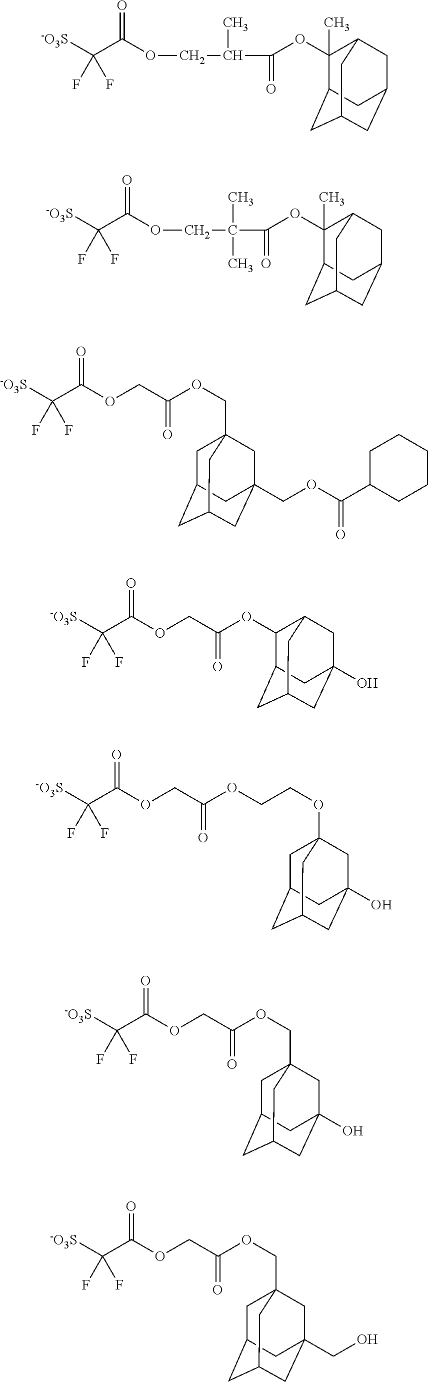

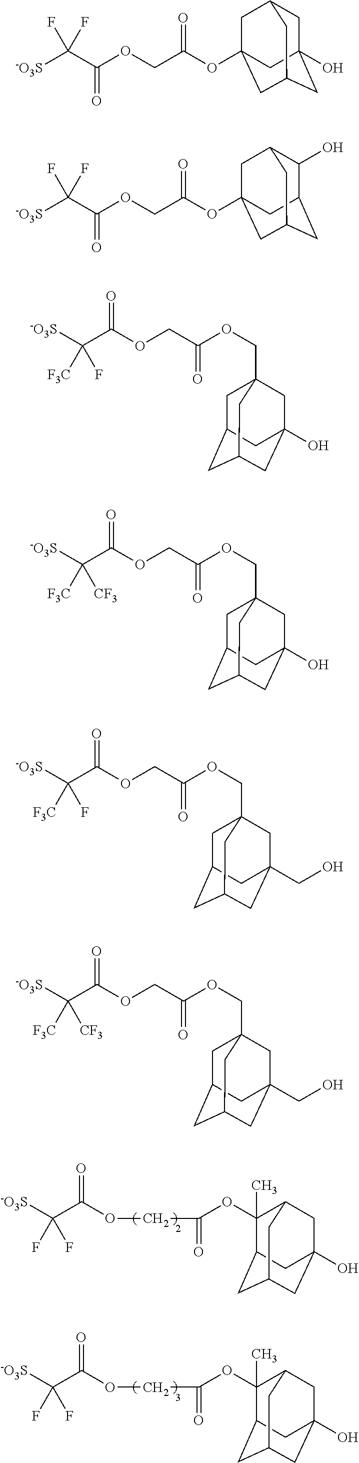

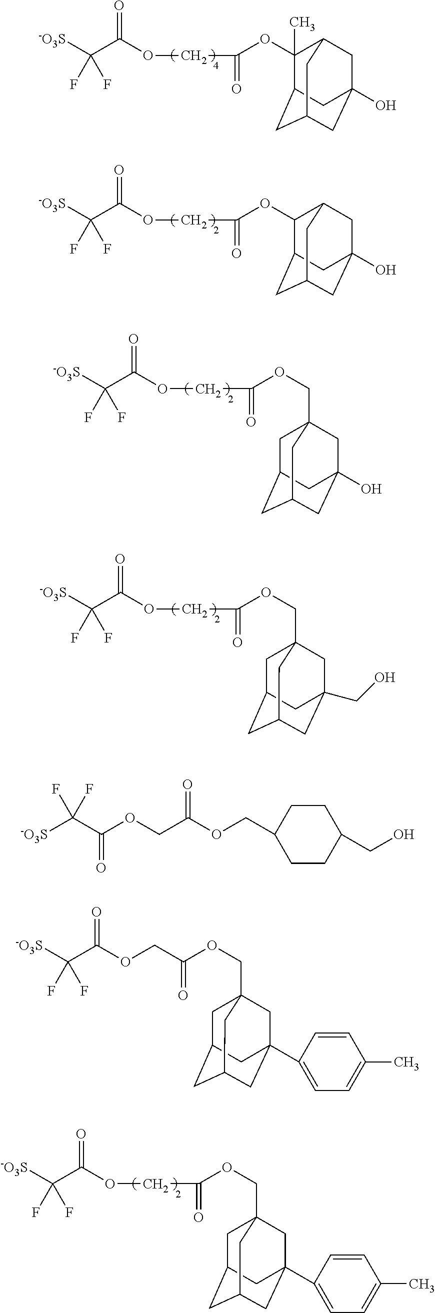

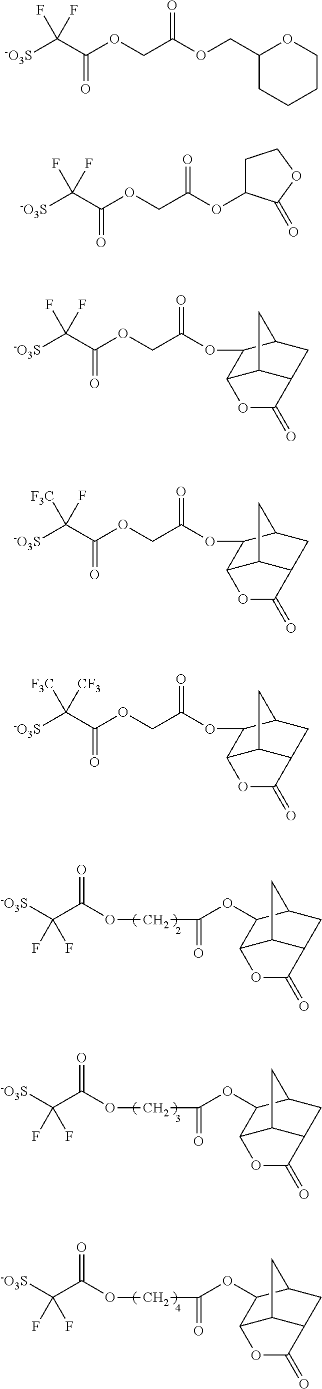

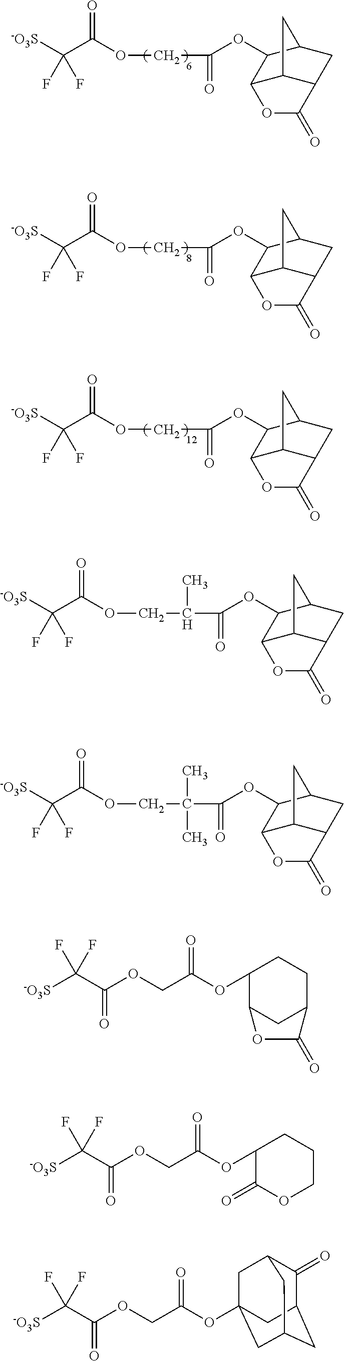

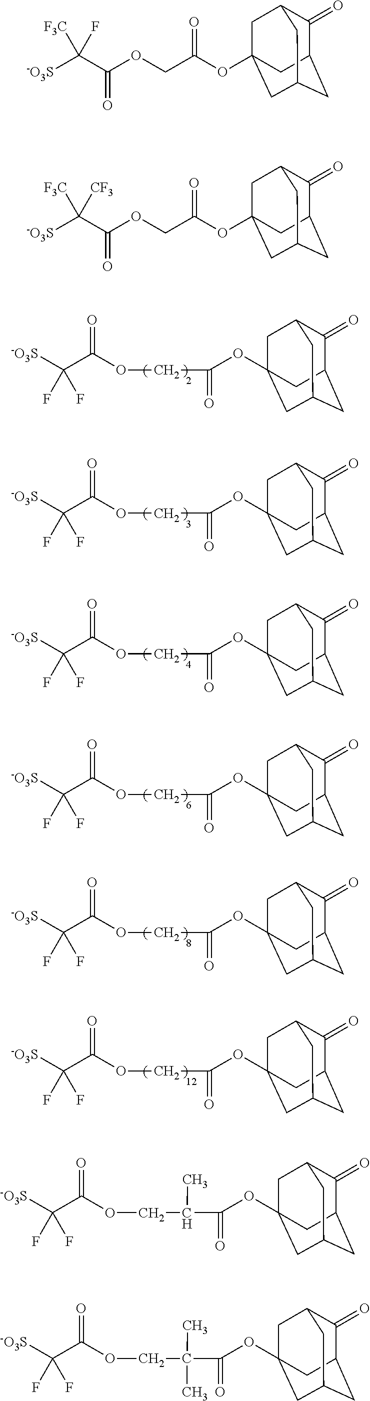

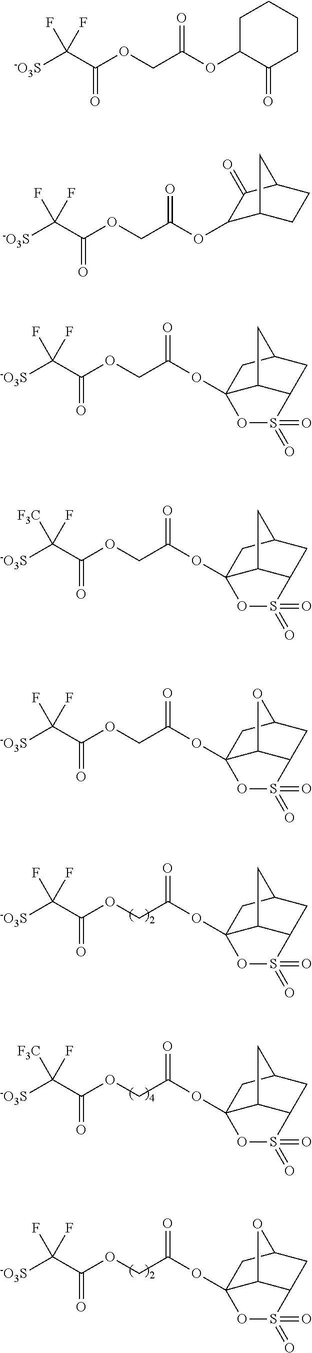

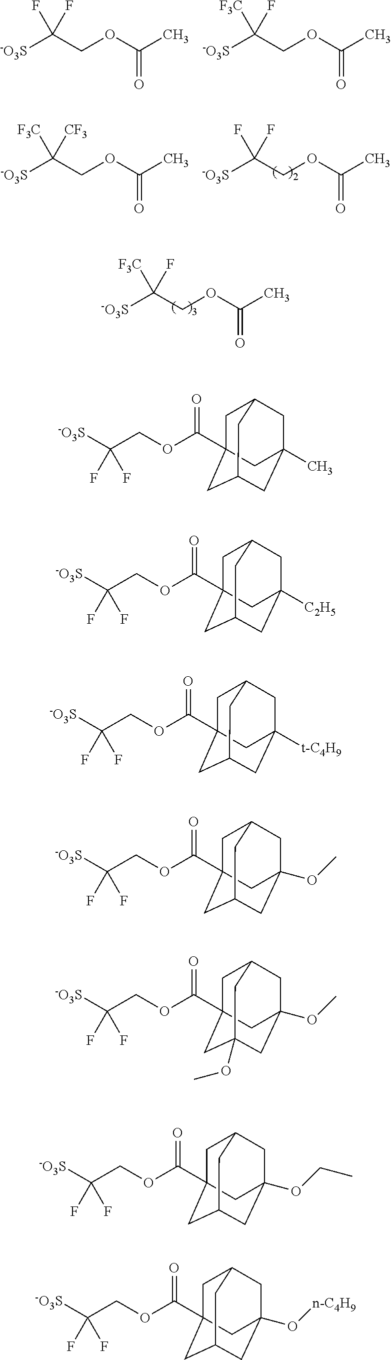

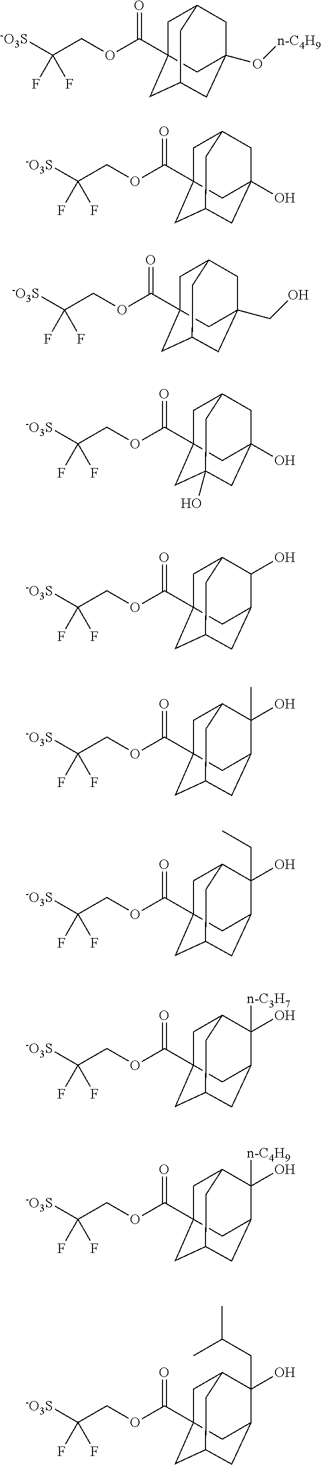

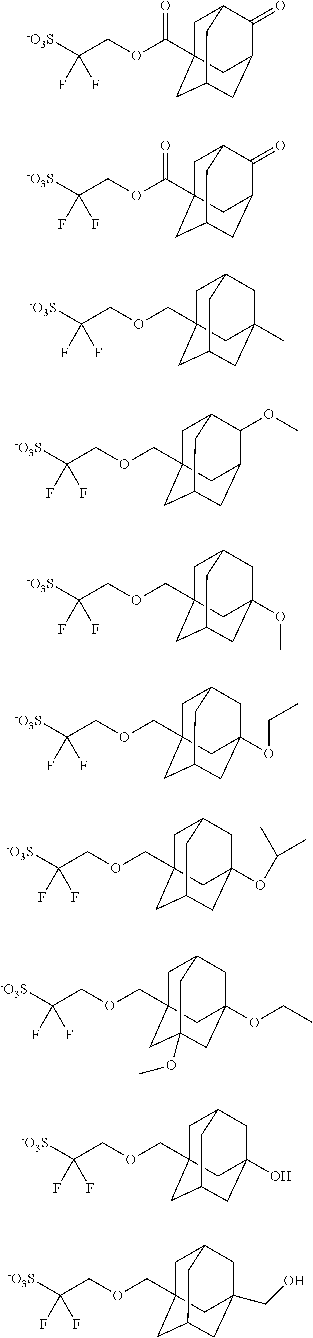

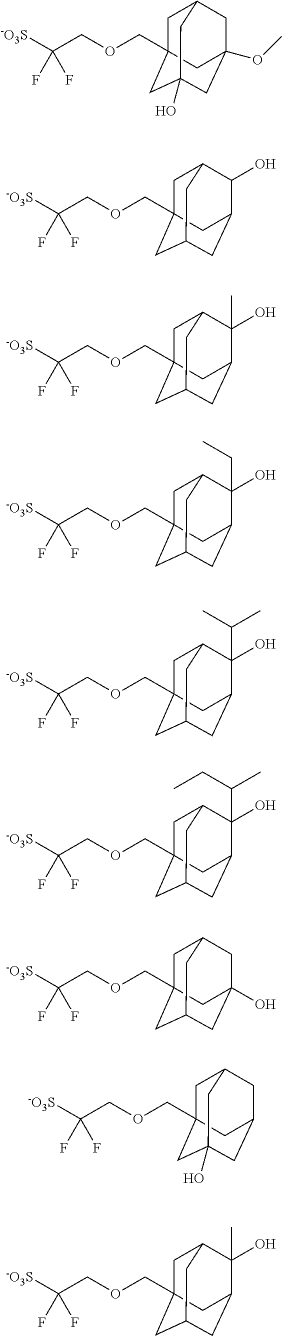

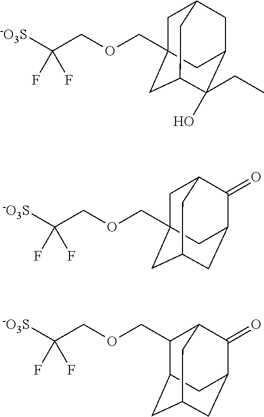

Among them, preferred are the following anions.

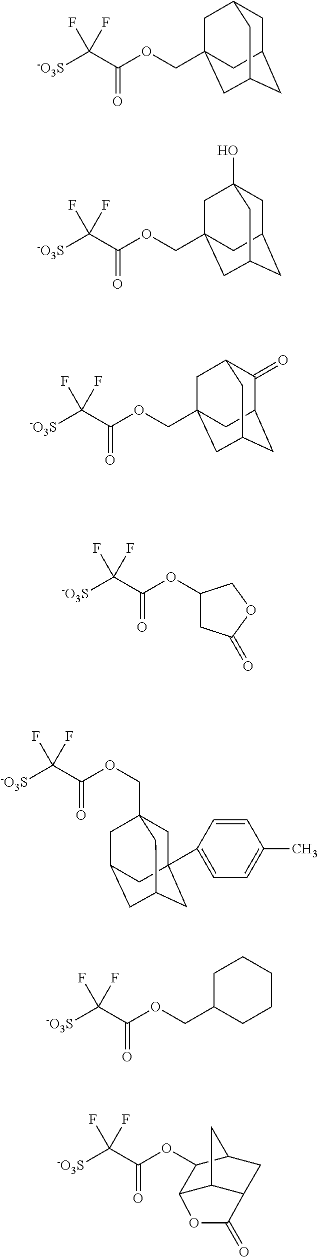

Examples of the cation part represented by Z⁺ of the salt represented by the formula (B1) include an onium cation such as a sulfonium cation, an iodonium cation, an ammonium cation, a benzothiazolium cation and a phosphonium cation. Among them, preferred are a sulfonium cation and an iodonium cation, and more preferred is an arylsulfonium cation.

Preferable examples of the cation part include the cations represented by the formulae (b2-1) to (b2-4):

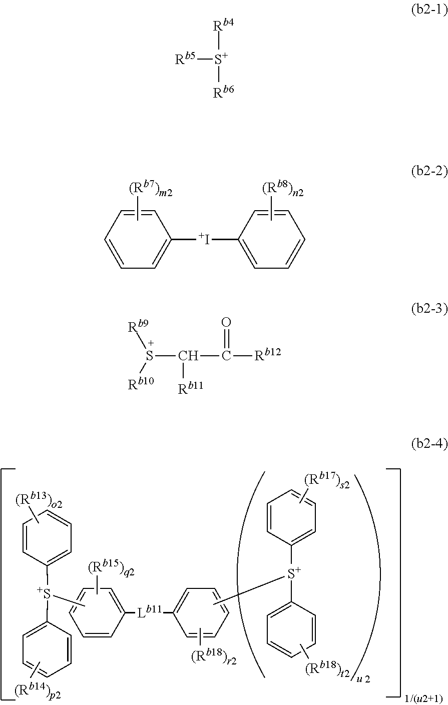

wherein R^{b4}, R^{b5} and R^{b6} independently represent a C1-C30 aliphatic hydrocarbon group which can have one or more substituents selected from the group consisting of a hydroxyl group, a C1-C12 alkoxy group and a C6-C18 aromatic hydrocarbon group, a C3-C18 saturated cyclic hydrocarbon group which can have one or more substituents selected from the group consisting of a halogen atom, a C2-C4 acyl group and a glycidyloxy group, or a C6-C18 aromatic hydrocarbon group which can have one or more substituents selected from the group consisting of a halogen atom, a hydroxyl group, a C1-C18 aliphatic hydrocarbon group, a C3-C18 saturated cyclic hydrocarbon group and a C1-C12 alkoxy group,
R^{b7} and R^{b8} are independently in each occurrence a hydroxyl group, a C1-C12 aliphatic hydrocarbon group or a C1-C12 alkoxy group, m2 and n2 independently represents an integer of 0 to 5,
R^{b9} and R^{b10} independently represent a C1-C18 aliphatic hydrocarbon group or a C3-C18 saturated cyclic hydrocarbon group, or R^{b9} and R^{b10} are bonded to form a C2-C11 divalent acyclic hydrocarbon group which forms a ring together with the adjacent S⁺, and one or more —CH₂— in the divalent acyclic hydrocarbon group may be replaced by —CO—, —O— or —S—,
and R^{b11} represents a hydrogen atom, a C1-C18 aliphatic hydrocarbon group, a C3-C18 saturated cyclic hydrocarbon group or a C6-C18 aromatic hydrocarbon group, R^{b12} represents a C1-C12 aliphatic hydrocarbon group, a C3-C18 saturated cyclic hydrocarbon group or a C6-C18 aromatic hydrocarbon group and the aromatic hydrocarbon group can have one or more substituents selected from the group consisting of a C1-C12 aliphatic hydrocarbon group, a C1-C12 alkoxy group, a C3-C18 saturated cyclic hydrocarbon group and a C2-C13 acyloxy group, or R^{b11} and R^{b12} are bonded each other to form a C1-C10 divalent acyclic hydrocarbon group which forms a 2-oxocycloalkyl group together with the adjacent —CHCO—, and one or more —CH₂— in the divalent acyclic hydrocarbon group may be replaced by —CO—, —O— or —S—, and
R^{b13}, R^{b14}, R^{b15}, R^{b16}, R^{b17} and R^{b18} independently represent a hydroxyl group, a C1-C12 aliphatic hydrocarbon group or a C1-C12 alkoxy group, L^{b11} represents —S— or —O— and o2, p2, s2 and t2 each independently represents an integer of 0 to 5, q2 and r2 each independently represents an integer of 0 to 4, and u2 represents 0 or 1.

The aliphatic hydrocarbon group represented by R^{b9} to R^{b11} has preferably 1 to 12 carbon atoms. The saturated cyclic hydrocarbon group represented by R^{b9} to R^{b11} has preferably 3 to 18 carbon atoms and more preferably 4 to 12 carbon atoms.

Preferable examples of the aliphatic hydrocarbon group include an alkyl group such as a methyl group, an ethyl group, a propyl group, an isopropyl group, a butyl group, a sec-butyl group, a tert-butyl group, a pentyl group, a hexyl group, an octyl group and a 2-ethylhexyl group. Preferable examples of the saturated cyclic hydrocarbon group include a cyclopropyl group, a cyclobutyl group, a cyclopentyl group, a cyclohexyl group, a cycloheptyl group, a cyclodecyl group, a 2-alkyl-a-adamantyl group, a 1-(1-adamantyl)-1-alkyl group and an isobornyl group. Preferable examples of the aromatic group include a phenyl group, a 4-methylphenyl group, a 4-ethylphenyl group, a 4-tert-butylphenyl group, a 4-cyclohexylphenyl group, a 4-methoxyphenyl group, a biphenyl group and a naphthyl group. Examples of the aliphatic hydrocarbon group having an aromatic hydrocarbon group include a benzyl group. Examples of the alkoxy group include a methoxy group, an ethoxy group, a propoxy group, an isopropoxy group, a butoxy group, a sec-butoxy group, a tert-butoxy group, a pentyloxy group, a hexyloxy group, a heptyloxy group, an octyloxy group, a 2-ethylhexyloxy group, a nonyloxy group, a decyloxy group, an undecyloxy group and a dodecyloxy group.

Examples of the C3-C12 divalent acyclic hydrocarbon group formed by bonding R^{b9} and R^{b10} include a trimethylene group, a tetramethylene group and a pentamethylene group. Examples of the ring group formed together with the adjacent S⁺ and the divalent acyclic hydrocarbon group include a thiolan-1-ium ring (tetrahydrothiphenium ring), a thian-1-ium ring and a 1,4-oxathian-4-iumring. A C3-C7 divalent acyclic hydrocarbon group is preferable.

Examples of the C1-C10 divalent acyclic hydrocarbon group formed by bonding R^{b11} and R^{b12} include a methylene group, an ethylene group, a trimethylene group, a tetramethylene group and a pentamethylene group and examples of the ring group include the followings.

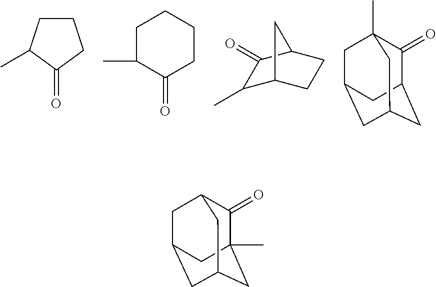

A C1-C5 divalent acyclic hydrocarbon group is preferable.

Among the above-mentioned cations, preferred is the cation represented by the formula (b2-1), and more preferred is the cation represented by the formula (b2-1-1). A triphenylsulfonium cation is especially preferable.

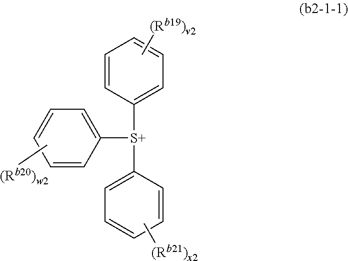

wherein R^{b19}, R^{b20} and R^{b21} are independently in each occurrence a halogen atom (preferably a fluorine atom), a hydroxyl group, a C1-C18 aliphatic hydrocarbon group, a C3-C18 saturated cyclic hydrocarbon group or a C1-C12 alkoxy group, and one or more hydrogen atoms of the aliphatic hydrocarbon group can be replaced by a hydroxyl group, a C1-C12 alkoxy group or a C6-C18 aromatic hydrocarbon group, and one or more hydrogen atoms of the saturated cyclic hydrocarbon group can be replaced by a halogen atom, a glycidyloxy group or a C2-C4 acyl group, and v2, w2 and x2 independently each represent an integer of 0 to 5.

The aliphatic hydrocarbon group has preferably 1 to 12 carbon atoms, and the saturated cyclic hydrocarbon group has preferably 4 to 18 carbon atoms, and v2, w2 and x2 independently each preferably represent 0 or 1.

It is preferred that R^{b19}, R^{b20} and R^{b21} are independently in each occurrence a halogen atom, a hydroxyl group, a C1-C12 alkyl group or a C1-C12 alkoxy group, and v2, w2 and x2 independently each represent an integer of 0 to 5. It is more preferred that R^{b19}, R^{b20} and R^{b21} are independently in each occurrence a fluorine atom, a hydroxyl group, a C1-C12 alkyl group or a C1-C12 alkoxy group, and v2, w2 and x2 independently each represent 0 or 1.

Examples of the cation represented by the formula (b2-1) include the following.

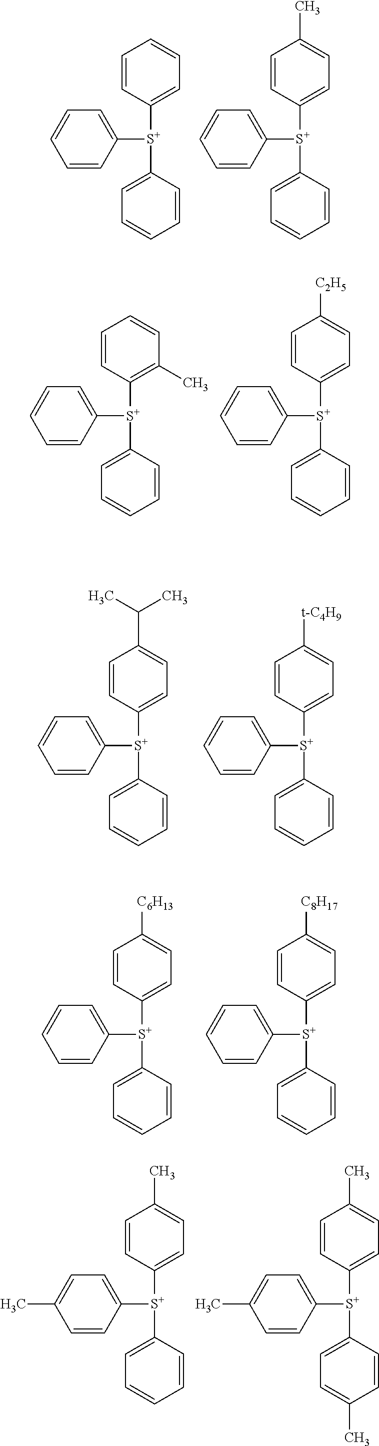

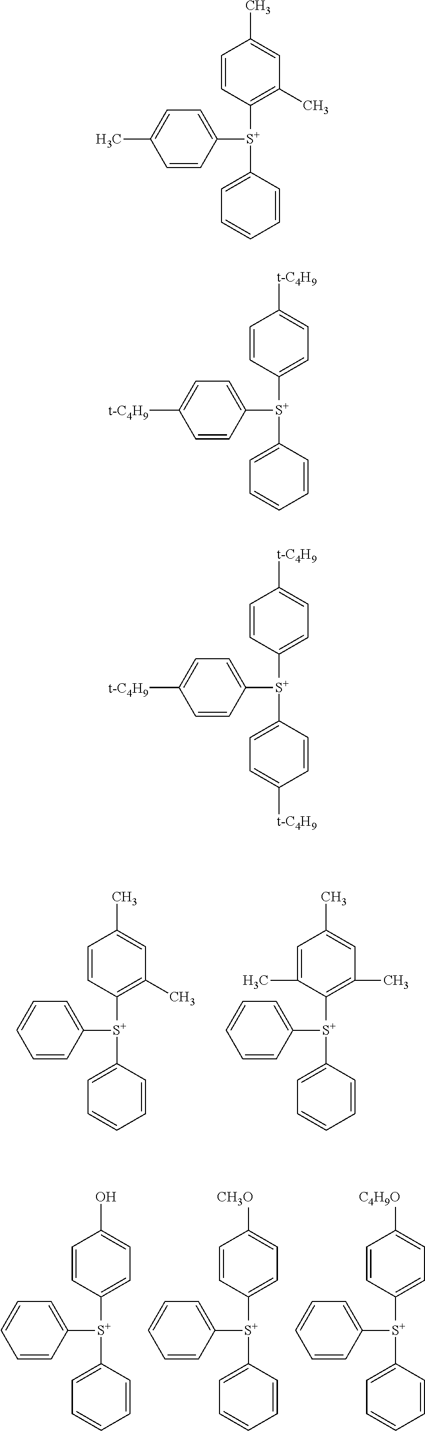

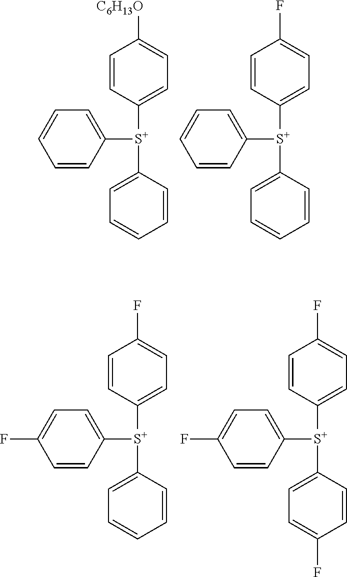

Examples of the cation represented by the formula (b2-2) include the followings.

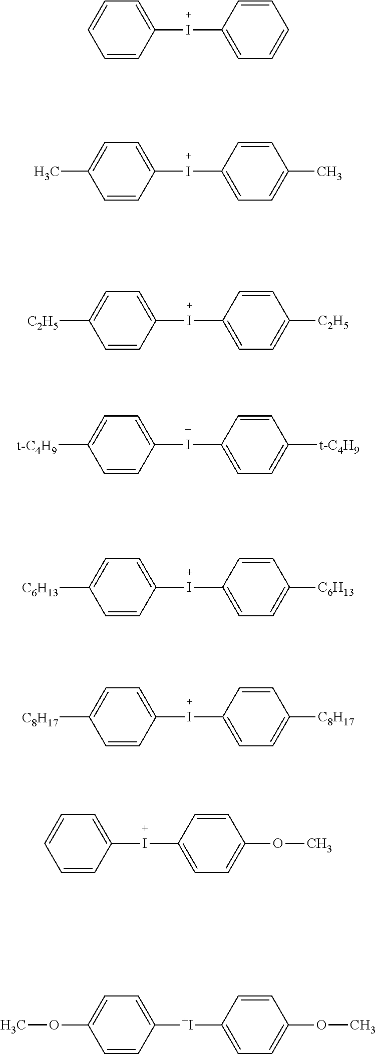

Examples of the cation represented by the formula (b2-3) include the followings.

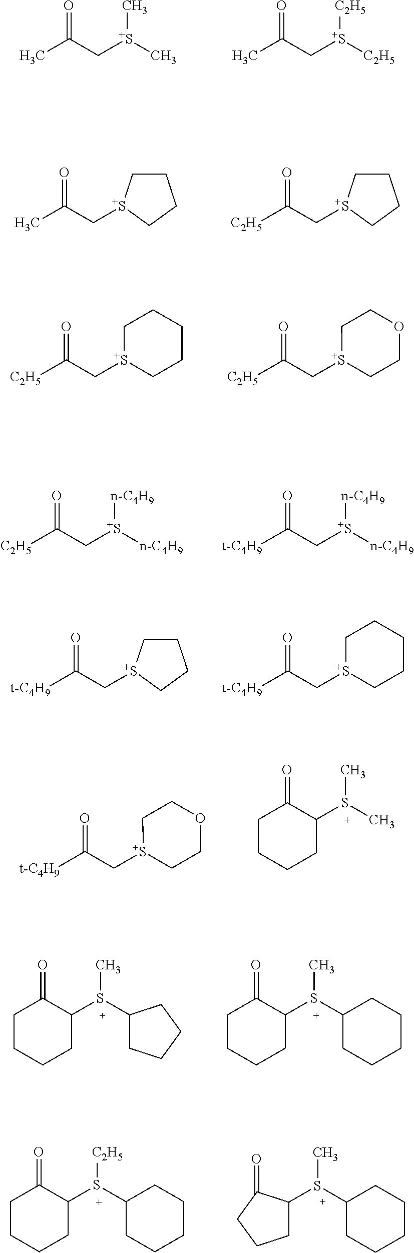

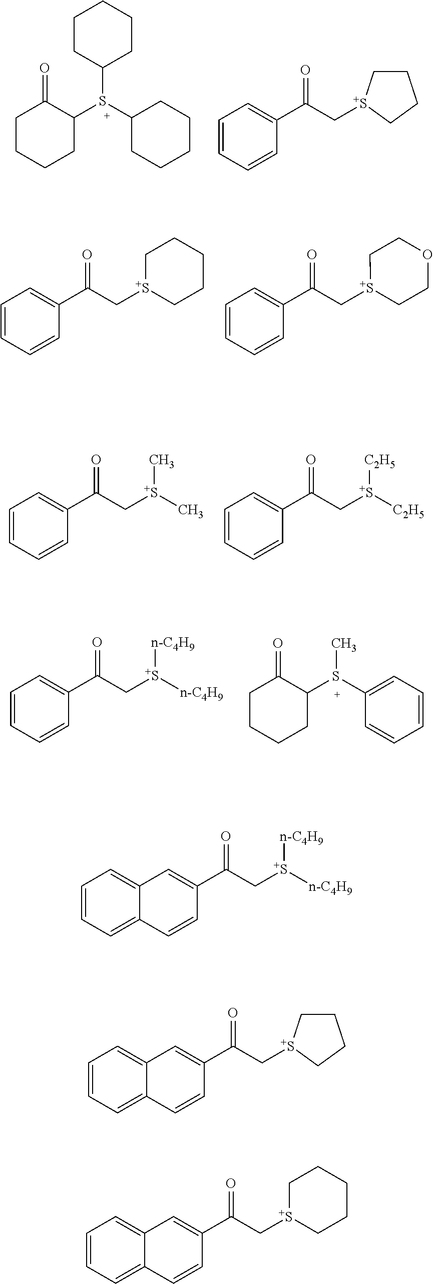

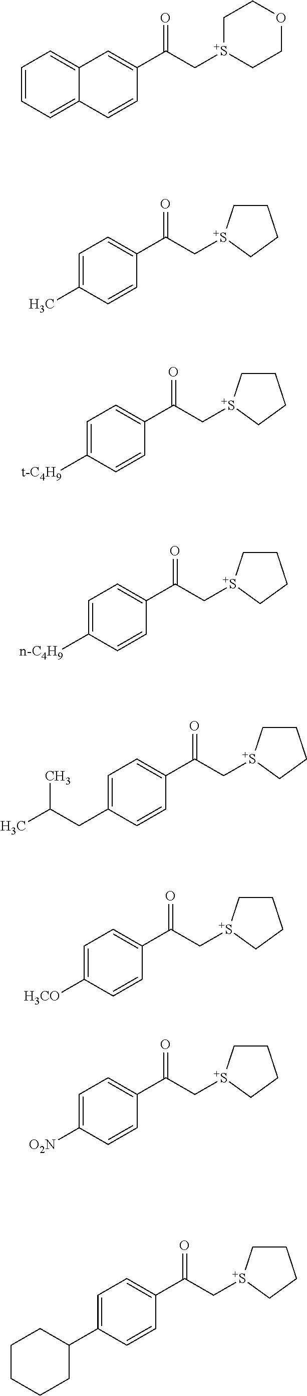

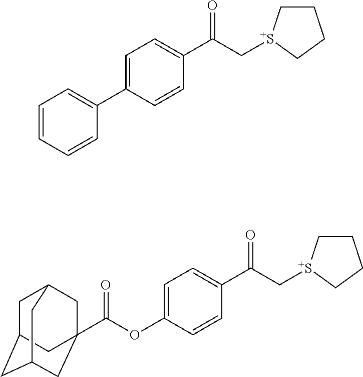

Examples of the cation represented by the formula (b2-4) include the followings.

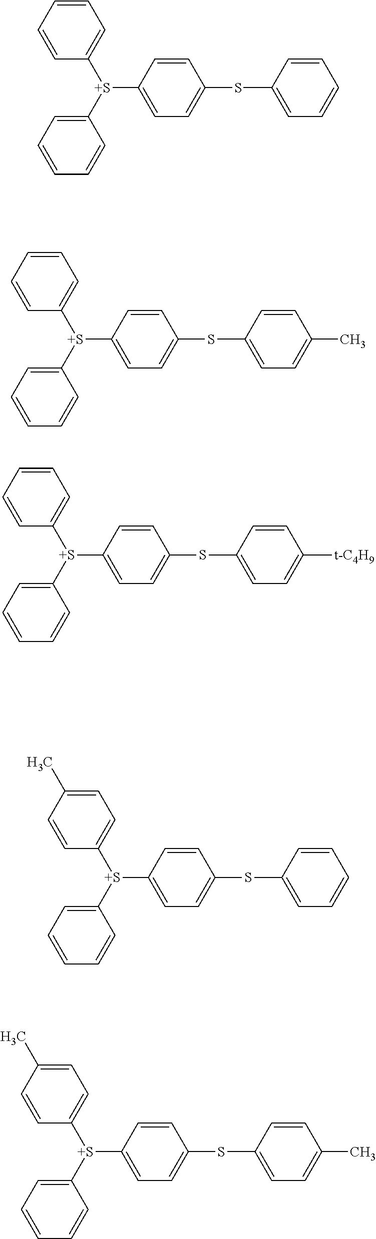

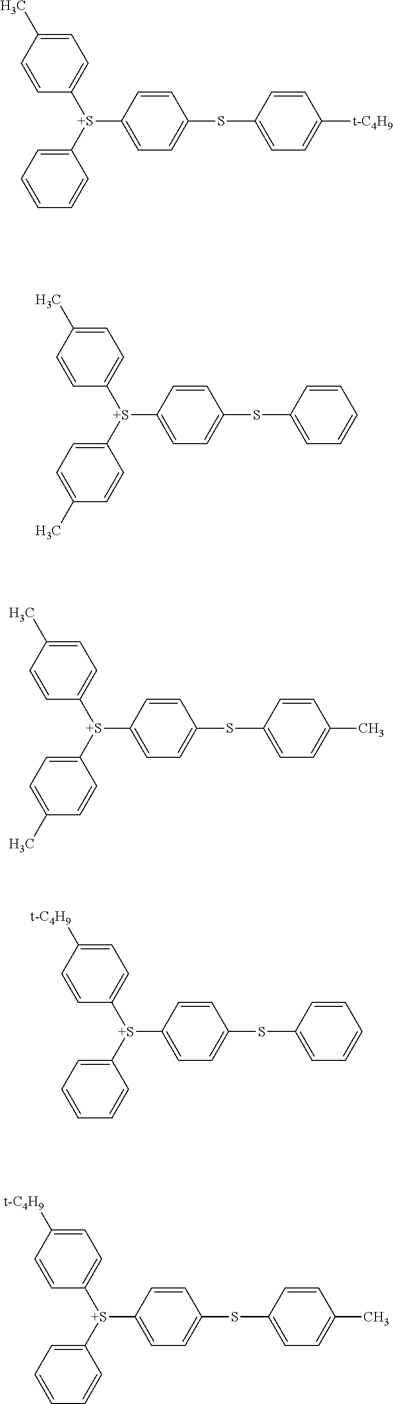

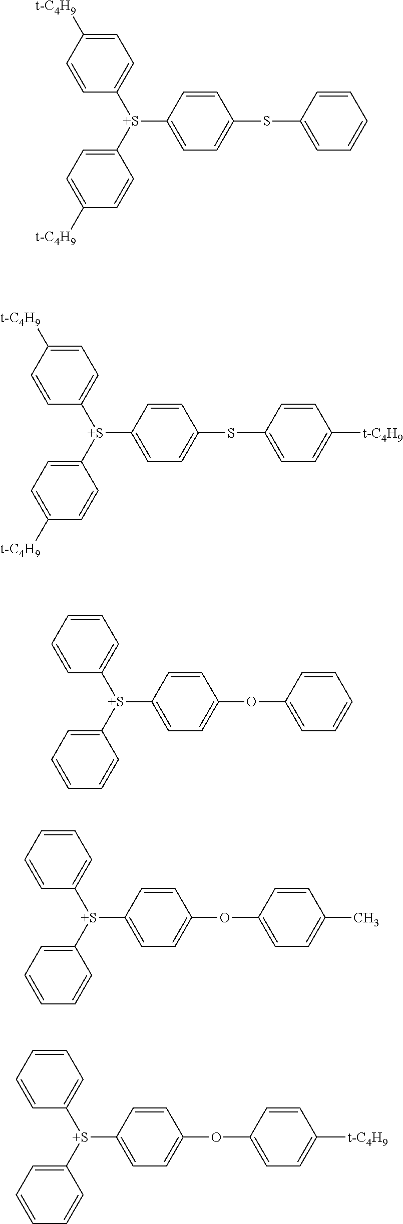

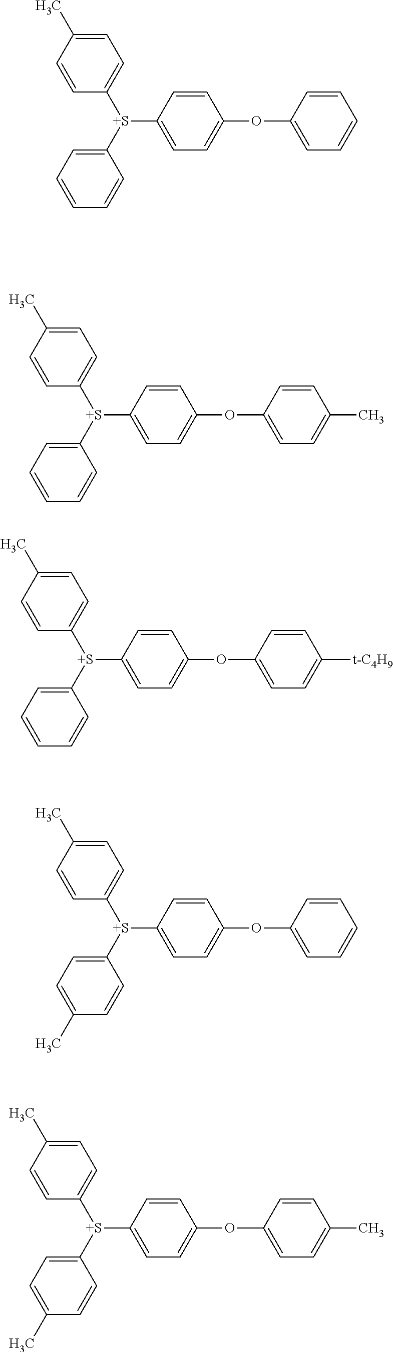

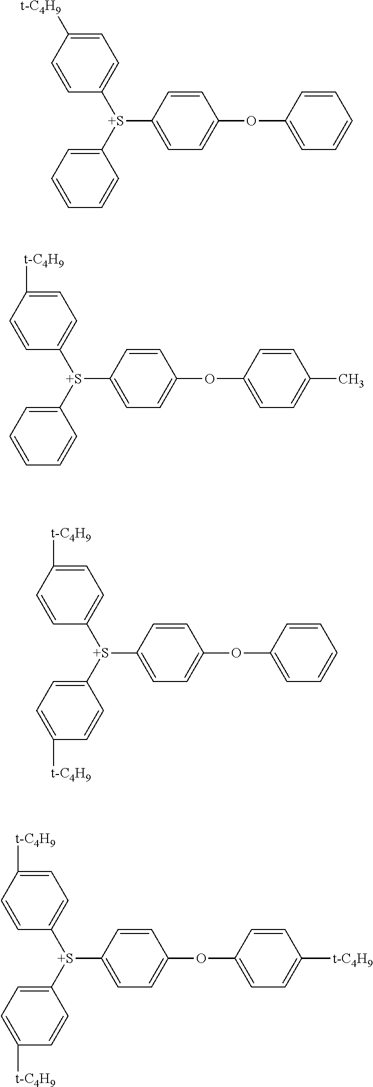

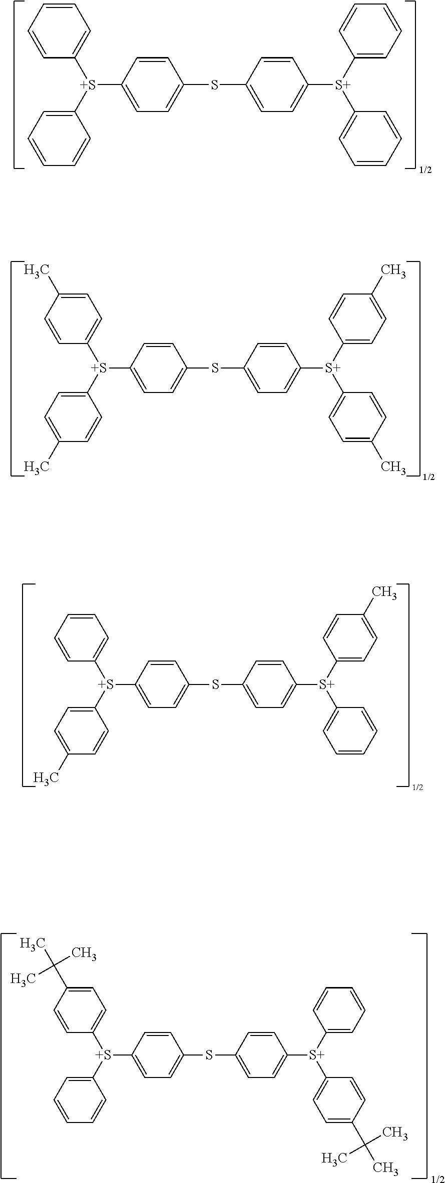

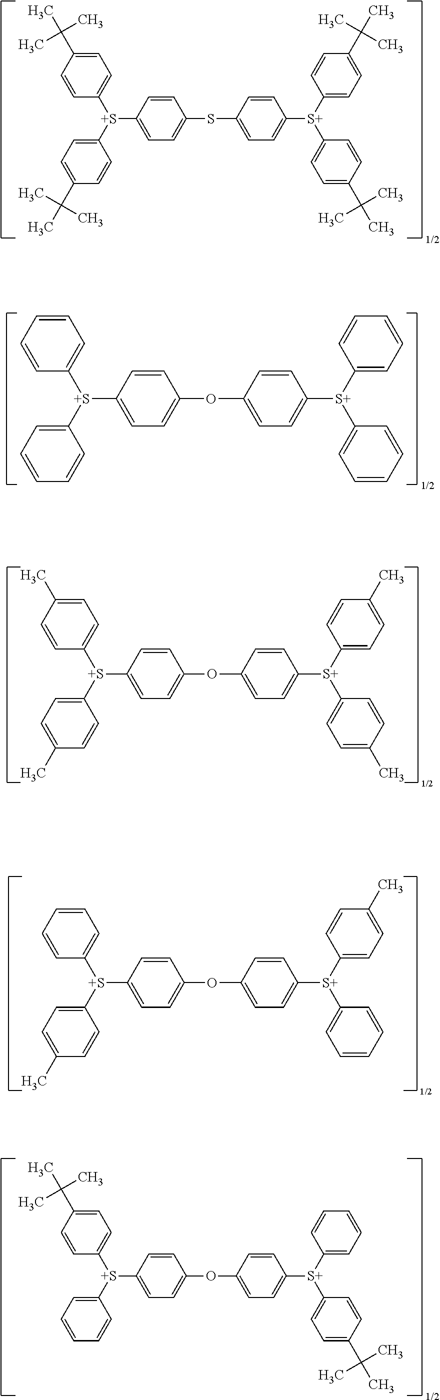

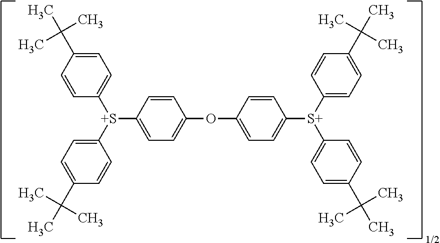

Examples of the salt represented by the formula (B1) include a salt wherein the anion is any one of the above-mentioned anions and the cation is any one of organic cations. Preferable examples of the salt include a combination of any one of anions represented by the formulae (b1-1-1) to (b1-1-9) and the cation represented by the formulae (b2-1-1), and a combination of any one of anions represented by the formulae (b1-1-3) to (b1-1-5) and the cation represented by the formulae (b2-3).

The salt represented by the formulae (B1-1) to (B1-17) are preferable, and the salt represented by the formulae (B1-1), (B1-2), (B1-6), (B1-11), (B1-12), (B1-13) and (B1-14) are more preferable.

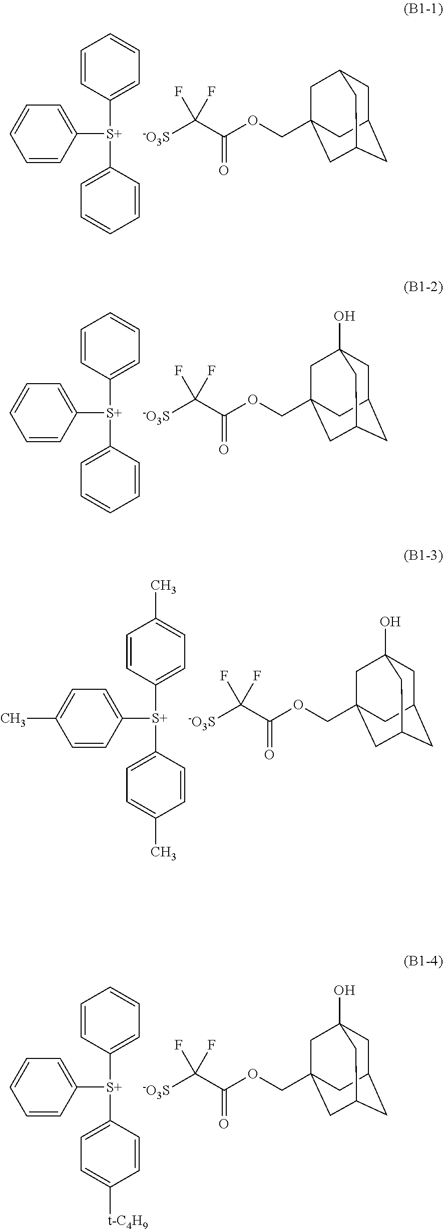

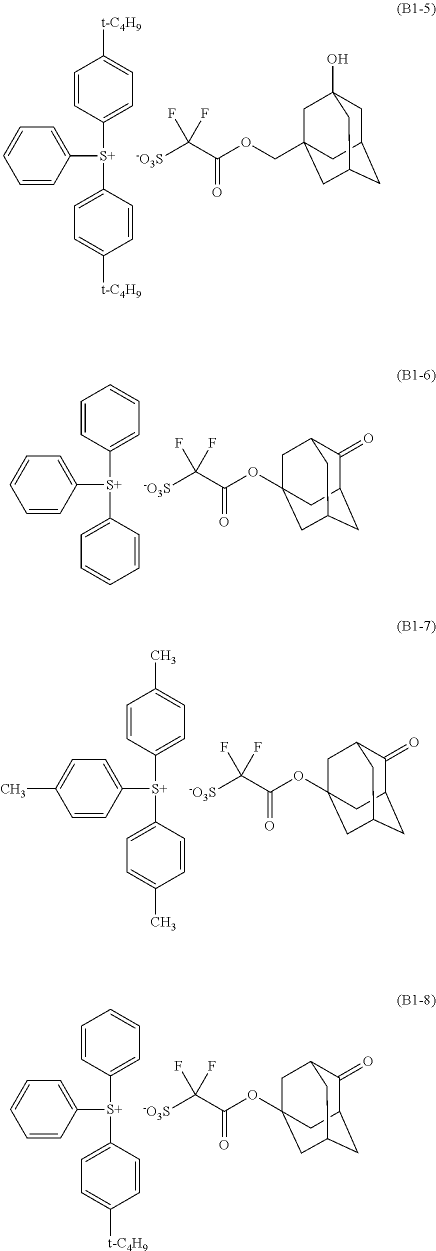

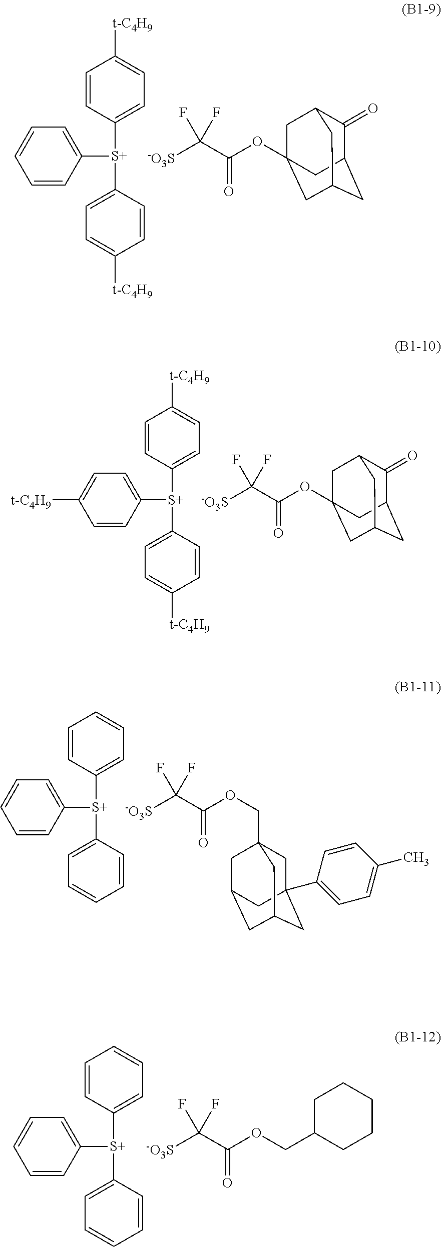

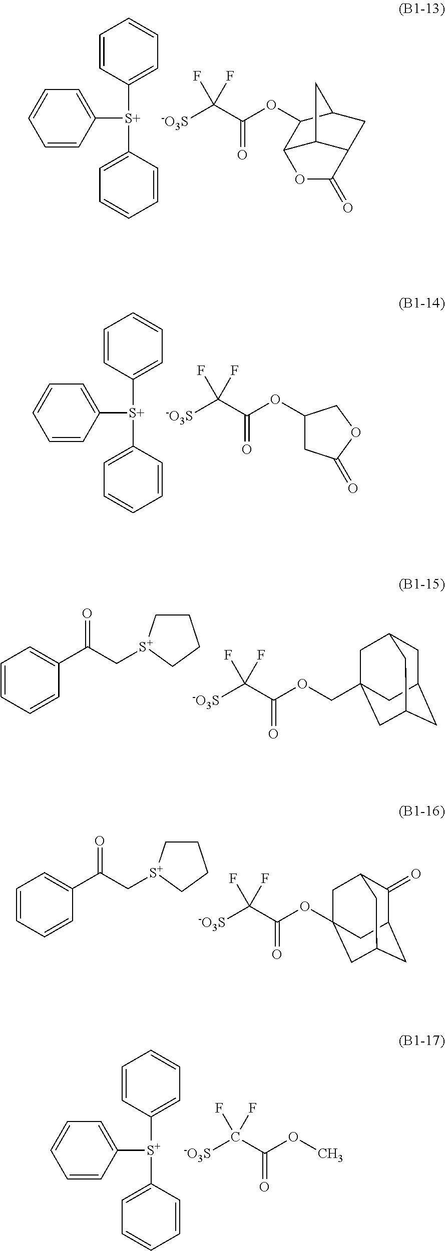

Two or more kinds of the acid generators can be used in combination.

The content of the acid generator in the photoresist composition of the present invention is usually 1 to 30 parts by weight and preferably 3 to 25 parts by weight per 100 parts by weight of the resin component.

In the present resist composition, performance deterioration caused by inactivation of acid which occurs due to post exposure delay can be diminished by adding a quencher.

Examples of the quencher include a compound represented by the formula (V):

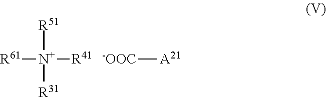

wherein R³¹, R⁴¹, R⁵¹ and R⁶¹ independently each represent a C1-C20 alkyl group which can have one or more substituents, a C3-C30 saturated cyclic hydrocarbon group which can have one or more substituents, or a C2-C20 alkenyl group which can have one or more substituents, and A²¹ represents a C1-C36 hydrocarbon group which can contain one or more heteroatoms and which have one or more substituents.

Examples of the C1-C20 alkyl group include a methyl group, an ethyl group, a propyl group, an isopropyl group, a butyl group, an isobutyl group, a sec-butyl group, a tert-butyl group, a pentyl group, an isopentyl group, a tert-pentyl group, a neopentyl group, a 1-methylbutyl group, a 2-methylbutyl group, a 1,2-dimethylpropyl group, a 1-ethylpropyl group, a hexyl group, a 1-methylpentyl group, a heptyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group and isocyl group, and a C1-C15 alkyl group is preferable, and C1-C10 alkyl group is more preferable.

Examples of the C3-C30 saturated cyclic hydrocarbon group include an adamantyl group, a norbornyl group, an isobornyl group, a tricyclodecyl group and a tetracyclodecyl group. The saturated cyclic hydrocarbon group preferably has 5 to 30 carbon atoms, more preferably 5 to 20 carbon atoms, much more preferably 6 to 15 carbon atoms and especially preferably 6 to 12 carbon atoms.

The alkenyl group preferably has 2 to 5 carbon atoms, and alkenyl group formed by combining the above-mentioned alkyl group with a vinyl group is more preferable.

Examples of the substituents include a halogen atom, a halogenated alkyl group such as a C1-C20 halogenated alkyl group, an alkyl group such as a C1-C20 alkyl group, an alkoxy group, a hydroxyalkoxy group, an alkoxyalkoxy group, an alkoxycarbonyloxy group, an alkoxycarbonylalkoxy group, an alkoxycarbonyl group, an aryl group, a heteroaryl group and an aralkyl group. Examples of the halogen atom include a fluorine atom, a chlorine atom, a bromine atom and an iodine atom, and a fluorine atom is preferable.

As the halogenated alkyl group, a fluorinated alkyl group is preferable. Examples of the alkyl group include the same as described in R⁶¹, R⁶², R⁶³ and R⁶⁴. Examples of aryl group include a phenyl group, a biphenyl group, a fluorenyl group, a naphthyl group, an anthryl group and a phenanthryl group. Examples of the heteroaryl group include the above-mentioned aryl groups in which one or more carbon atoms composed of the aromatic ring is replaced by a heteroatom such as an oxygen atom, a sulfur atom and a nitrogen atom. Examples of the aralkyl group include a benzyl group, a phenethyl group, a 1-naphthylmethyl group, a 2-naphthylmethyl group, a 1-naphthylethyl group and a 2-naphthylethyl group. As the aralkyl group, an aryl-substitued C1-C4 alkyl group is preferable, and an aryl-substitued C1-C2 alkyl group is more preferable, and an aryl-substitued methyl group is especially preferable. The aryl group, the heteroaryl group and the aralkyl group can have one or more substituents such as a C1-C10 alkyl group, a halogenated alkyl group (e.g. a C1-C8 halogenated alkyl group), an alkoxy group, a hydroxyl group and a halogen atom.

It is preferred that R³¹, R⁴¹, R⁵¹ and R⁶¹ independently each represent a linear alkyl group, a linear alkenyl group, or a saturated cyclic hydrocarbon group, and it is more preferred that R³¹, R⁴¹, R⁵¹ and R⁶¹ independently each represent a linear alkyl group. It is preferred that one of R³¹, R⁴¹, R⁵¹ and R⁶¹ represents an alkyl group having 1 to 4 carbon atoms.

Examples of the C1-C36 hydrocarbon group represented by A²¹ include a saturated hydrocarbon group, an unsaturated hydrocarbon group, an aromatic hydrocarbon group and an aralkyl group. Examples of the saturated hydrocarbon group include a C1-C20 alkyl group and a C3-C20 saturated cyclic hydrocarbon group which are described in R⁶¹, R⁶², R⁶³ and R⁶⁴. The unsaturated hydrocarbon group preferably has 2 to 5 carbon atoms, more preferably 2 to 4 carbon atoms, and especially has 3 carbon atoms. Examples of the unsaturated hydrocarbon group include a vinyl group, a propenyl group, a butynyl group, a 1-methylpropenyl group, and a 2-methylpropenyl group, and a propenyl group is preferable. The aromatic hydrocarbon group preferably has 6 to 36 carbon atoms, more preferably 6 to 30 carbon atoms, much more preferably 6 to 20 carbon atoms, and especially preferably 6 to 15 carbon atoms. Examples of the aromatic hydrocarbon group include an aryl group such as a phenyl group, a biphenyl group, a fluorenyl group, a naphthyl group, an anthryl group and a phenanthryl group. Examples of the aralkyl group include a benzyl group, a phenethyl group, a 1-naphthylmethyl group, a 2-naphthylmethyl group, a 1-naphthylethyl group and a 2-naphthylethyl group. As the aralkyl group, an aryl-substitued C1-C4 alkyl group is preferable, and an aryl-substitued C1-C2 alkyl group is more preferable, and an aryl-substitued methyl group is especially preferable.

The C1-C36 hydrocarbon group can have one or more substituents, and examples of the substituents include an alkyl group, an aryl group, an alkoxy group, a halogen atom, a halogenated alkyl group, a hydroxyl group and an oxo group (═O), and a halogen atom and a hydroxyl group are preferable, and a hydroxyl group is more preferable. The C1-C36 hydrocarbon group can contain one or more heteroatoms such as an oxygen atom, a sulfur atom and a nitrogen atom. Examples of the alkyl group include a C1-C5 alkyl group such as a methyl group, an ethyl group, a propyl group, a butyl group and a tert-butyl group, and examples of the aryl group include the same as described above. Examples of the alkoxy group include a C1-C5 alkoxy group such as a methoxy group, an ethoxy group, a propoxy group, a butoxy group and a tert-butoxy group, and methoxy and ethoxy groups are preferable. Examples of the halogen atom include a fluorine atom, a chlorine atom, a bromine atom and an iodine atom, and a fluorine atom is preferable.

Preferable examples of the compound represented by the formula (V) include a compound represented by the formula (IV):

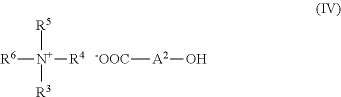

wherein R³, R⁴, R⁵ and R⁶ independently each represent a C1-C6 alkyl group and A² represents a C3-C36 divalent saturated cyclic hydrocarbon group which can contain one or more heteroatoms and which have one or more substituents or a C6-C20 divalent aromatic hydrocarbon group which can contain one or more heteroatoms and which have one or more substituents.

Examples of the C1-C6 alkyl group include a methyl group, an ethyl group, a propyl group, an isopropyl group, a butyl group, an isobutyl group, a sec-butyl group, a tert-butyl group, a pentyl group, an isopentyl group, a tert-pentyl group, a neopentyl group, a 1-methylbutyl group, a 2-methylbutyl group, a 1,2-dimethylpropyl group, a 1-ethylpropyl group, a hexyl group, a 1-methylpentyl group and a heptyl group.

Examples of the C3-C36 divalent saturated cyclic hydrocarbon group include a C3-C8 cycloalkanediyl group such as a cyclopropanediyl group, a cyclobutanediyl group, a cyclopentanediyl group, a cyclohexanediyl group, a methylcyclohexanediyl group, a cycloheptanediyl group and a cyclooctanediyl group, a C5-C12 cycloalkylalkane-diyl group such as a cyclobutylmethane-diyl group, a cyclopentylmethane-diyl group, a cyclohexylmethane-diyl group, a cycloheptylmethane-diyl group and a cyclooctylmethane-diyl group, and an adamantanediyl group and a 1-asamantylmethane-diyl group.

Examples of the C6-C20 divalent aromatic hydrocarbon group include a phenylene group which can have one or more alkyl groups such as a phenylene group, a methylphenylene group, an ethylphenylene group, a tert-butylphenylene group and a dimethylphenylene group, and a naphthylene group which can have one or more alkyl groups such as a naphthylene group and a methylnaphthylene group.

Examples of the C3-C36 divalent saturated cyclic hydrocarbon group containing one or more heteroatoms include a pyrrolidinediyl group, a pyrazolidinediyl group, an imidazolidinediyl group, an isooxazolidinediyl group, an isothiazolidinediyl group, a piperidinediyl group, a piperazinediyl group, a morpholinediyl group, a thiomorpholinediyl group, a diazolediyl group, a triazolediyl group and a tetrazolediyl group. Examples of the C6-C20 divalent aromatic hydrocarbon group containing one or more heteroatoms include a pyridinediyl group and a bipyridinediyl group.

Examples of the substituents include a halogen atom, a hydroxyl group, an amino group, a mercapto group (—SH), a hydrocarbon group having 30 or less carbon atoms, a heterocyclic group and an oxo group (═O).

Examples of the cation parts of the compounds represented by the formulae (IV) and (V) include the cations represented by the formulae (IA-1) to (IA-8):

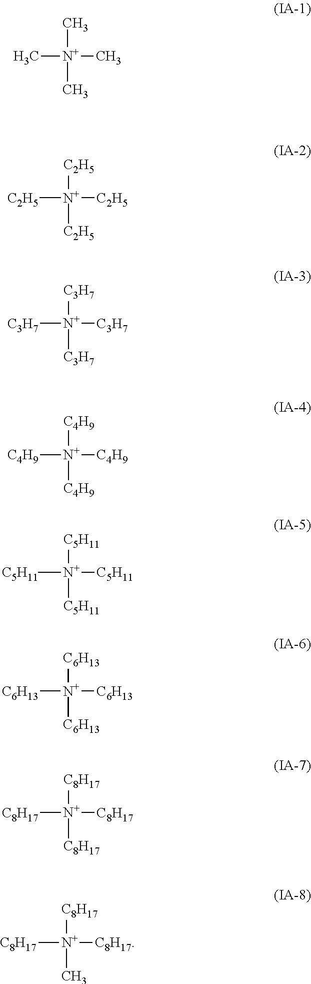

Examples of the anion parts of the compounds represented by the formulae (IV) and (V) include the anions represented by the formulae (IB-1) to (IB-11):

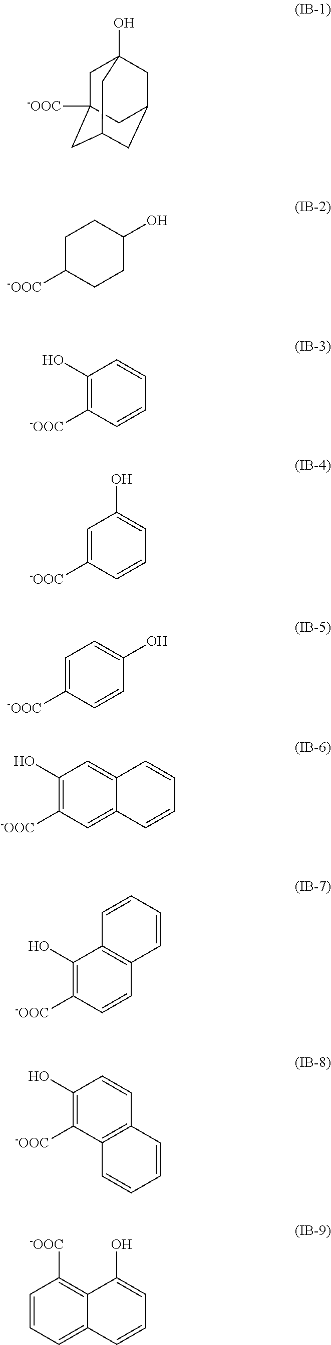

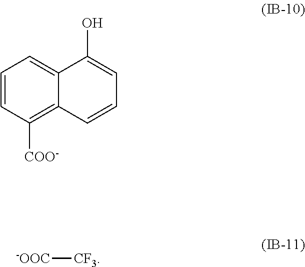

Examples of the compounds represented by the formulae (IV) and (V) include compounds Nos. (IV-1) to (IV-35) as shown in Table 1 and Table 2. Among them, preferred are compounds Nos. (IV-1) to (IV-5) and (IV-12) to (IV-16), and more preferred are compound Nos. (IV-12) to (IV-16).

TABLE 1

| Compound No. | Cation | Anion |
|---|---|---|
| (IV-1) | (IA-1) | (IB-1) |
| (IV-2) | (IA-1) | (IB-2) |
| (IV-3) | (IA-1) | (IB-3) |
| (IV-4) | (IA-1) | (IB-4) |
| (IV-5) | (IA-1) | (IB-5) |
| (IV-6) | (IA-2) | (IB-1) |
| (IV-7) | (IA-2) | (IB-2) |
| (IV-8) | (IA-2) | (IB-3) |
| (IV-9) | (IA-3) | (IB-1) |
| (IV-10) | (IA-3) | (IB-3) |
| (IV-11) | (IA-3) | (IB-5) |
| (IV-12) | (IA-4) | (IB-1) |
| (IV-13) | (IA-4) | (IB-2) |

TABLE 2

| Compound No. | Cation | Anion |
|---|---|---|
| (IV-14) | (IA-4) | (IB-3) |
| (IV-15) | (IA-4) | (IB-4) |
| (IV-16) | (IA-4) | (IB-5) |
| (IV-17) | (IA-5) | (IB-1) |
| (IV-18) | (IA-5) | (IB-3) |
| (IV-19) | (IA-6) | (IB-1) |
| (IV-20) | (IA-6) | (IB-3) |
| (IV-21) | (IA-4) | (IB-6) |
| (IV-22) | (IA-4) | (IB-7) |
| (IV-23) | (IA-4) | (IB-8) |
| (IV-24) | (IA-4) | (IB-9) |
| (IV-25) | (IA-4) | (IB-10) |
| (IV-26) | (IA-5) | (IB-8) |
| (IV-27) | (IA-6) | (IB-8) |
| (IV-28) | (IA-7) | (IB-1) |
| (IV-29) | (IA-7) | (IB-3) |
| (IV-30) | (IA-7) | (IB-8) |
| (IV-31) | (IA-8) | (IB-11) |

The compounds represented by the formula (IV) and (V) can be produced, for example, by reacting tetraalkylammonium hydroxide such as tetramethylammonium hydroxide with hydroxyalkanecarboxlic acid such as hydroxyadamantanecarbozylic acid.

Two or more kinds of the compounds represented by the formula (IV) and (V) can be used in combination.

The content of the compound represented by the formula (IV) or (V) is usually 0.01 to 10% by weight, preferably 0.05 to 8% by weight and more preferably 0.01 to 5% by weight based on solid component.

Examples of the quencher include a basic compound. The content of the basic compound is usually 0.01 to 1% by weight based on solid component.

The basic compound is preferably an organic base compound, and more preferably a nitrogen-containing organic base compound.

Examples thereof include an amine compound such as an aliphatic amine and an aromatic amine and an ammonium salt. Examples of the aliphatic amine include a primary amine, a secondary amine and a tertiary amine. Examples of the aromatic amine include an aromatic amine in which aromatic ring has one or more amino groups such as aniline and a heteroaromatic amine such as pyridine. Preferable examples thereof include an aromatic amine represented by the formula (C2):

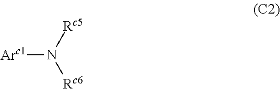

wherein Ar^{c1} represents an aromatic hydrocarbon group, and R^{c5} and R^{c6} each independently represent a hydrogen atom, an aliphatic hydrocarbon group, a saturated cyclic hydrocarbon group or an aromatic hydrocarbon group, and the aliphatic hydrocarbon group, the saturated cyclic hydrocarbon group and the aromatic hydrocarbon group can have one or more substituents selected from the group consisting of a hydroxyl group, an amino group, an amino group having one or two C1-C4 alkyl groups and a C1-C6 alkoxy group.

The aliphatic hydrocarbon group is preferably an alkyl group and the saturated cyclic hydrocarbon group is preferably a cycloalkyl group. The aliphatic hydrocarbon group preferably has 1 to 6 carbon atoms. The saturated cyclic hydrocarbon group preferably has 5 to 10 carbon atoms. The aromatic hydrocarbon group preferably has 6 to 10 carbon atoms.

As the aromatic amine represented by the formula (C2), an amine represented by the formula (C2-1):

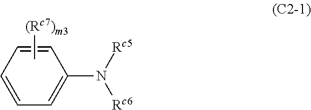

wherein R^{c5} and R⁵⁶ are the same as defined above, and R^{c7} is independently in each occurrence an aliphatic hydrocarbon group, an alkoxy group, a saturated cyclic hydrocarbon group or an aromatic hydrocarbon group, and the aliphatic hydrocarbon group, the alkoxy group, the saturated cyclic hydrocarbon group and the aromatic hydrocarbon group can have one or more substituents selected from the group consisting of a hydroxyl group, an amino group, an amino group having one or two C1-C4 alkyl groups and a C1-C6 alkoxy group, and m3 represents an integer of 0 to 3, is preferable. The aliphatic hydrocarbon group is preferably an alkyl group and the saturated cyclic hydrocarbon group is preferably a cycloalkyl group. The aliphatic hydrocarbon group preferably has 1 to 6 carbon atoms.

The saturated cyclic hydrocarbon group preferably has 5 to 10 carbon atoms. The aromatic hydrocarbon group preferably has 6 to 10 carbon atoms. The alkoxy group preferably has 1 to 6 carbon atoms.

An ammonium salt represented by the formula (C2-2):

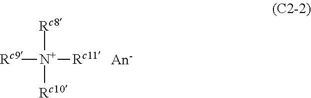

wherein R^{c8′}, R^{c9′}, R^{c10′}, and R^{c11′} each independently represent an aliphatic hydrocarbon group, a saturated cyclic hydrocarbon group or an aromatic hydrocarbon group, and the aliphatic hydrocarbon group, the saturated cyclic hydrocarbon group and the aromatic hydrocarbon group can have one or more substituents selected from the group consisting of a hydroxyl group, an amino group, an amino group having one or two C1-C4 alkyl groups and a C1-C6 alkoxy group, and An⁻ represents OH⁻, is also preferable. The aliphatic hydrocarbon group is preferably an alkyl group and the saturated cyclic hydrocarbon group is preferably a cycloalkyl group. The aliphatic hydrocarbon group preferably has 1 to 8 carbon atoms. The saturated cyclic hydrocarbon group preferably has 5 to 10 carbon atoms. The aromatic hydrocarbon group preferably has 6 to 10 carbon atoms.

The alkoxy group preferably has 1 to 6 carbon atoms.

Examples of the aromatic amine represented by the formula (C2) include 1-naphthylamine, 2-naphthylamine, aniline,

diisopropylaniline, 2-methylaniline, 3-methylaniline, 4-methylaniline, 4-nitroaniline, N-methylaniline, N,N-dimethylaniline, and diphenylamine, and among them, preferred is diisopropylaniline and more preferred is 2,6-diisopropylaniline.

Examples of the ammonium salt represented by the formula (C2-2) include tetramethylammonium hydroxide and tetrabutylammonium hydroxide.

Other examples of the basic compound include amines represented by the formulae (C3) to (C11):

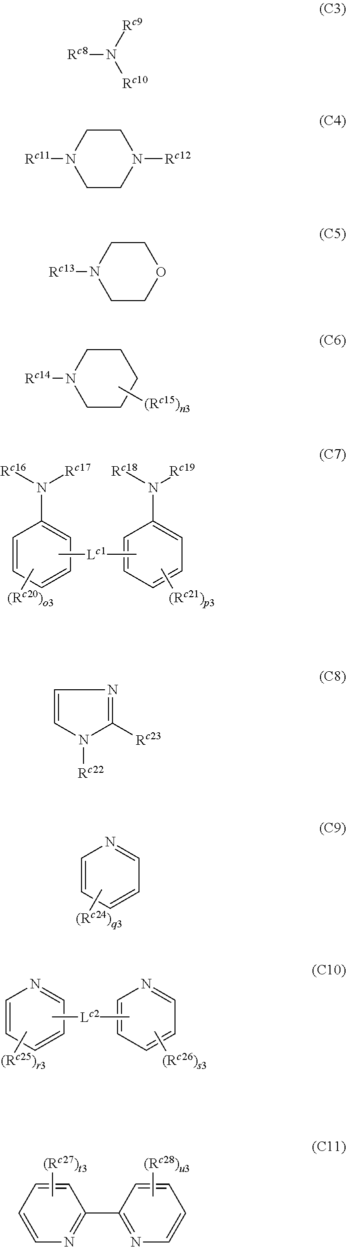

wherein R^{c8}, R^{c20}, R^{c21}, and R^{c23} to R^{c28} each independently represent an aliphatic hydrocarbon group, an alkoxy group, a saturated cyclic hydrocarbon group or an aromatic hydrocarbon group, and the aliphatic hydrocarbon group, the alkoxy group, the saturated cyclic hydrocarbon group and the aromatic hydrocarbon group can have one or more substituents selected from the group consisting of a hydroxyl group, an amino group, an amino group having one or two C1-C4 alkyl groups and a C1-C6 alkoxy group,
R^{c9}, R^{c10}, R^{c11} to R^{c14}, R^{c16} to R^{c19}, and R^{c22} each independently represents a hydrogen atom, an aliphatic hydrocarbon group, a saturated cyclic hydrocarbon group or an aromatic hydrocarbon group, and the aliphatic hydrocarbon group, the saturated cyclic hydrocarbon group and the aromatic hydrocarbon group can have one or more substituents selected from the group consisting of a hydroxyl group, an amino group, an amino group having one or two C1-C4 alkyl groups and a C1-C6 alkoxy group,
R^{c15} is independently in each occurrence an aliphatic hydrocarbon group, a saturated cyclic hydrocarbon group or an alkanoyl group, L^{c1} and L^{c2} each independently represents a divalent aliphatic hydrocarbon group, —CO—, —C(═NH)—, —C(═NR^{c3})—, —S—, —S—S— or a combination thereof and R^{c3} represents a C1-C4 alkyl group, O3 to u3 each independently represents an integer of 0 to 3 and n3 represents an integer of 0 to 8.

The aliphatic hydrocarbon group has preferably 1 to 6 carbon atoms, and the saturated cyclic hydrocarbon group has preferably 3 to 6 carbon atoms, and the alkanoyl group has preferably 2 to 6 carbon atoms, and the divalent aliphatic hydrocarbon group has preferably 1 to 6 carbon atoms. The divalent aliphatic hydrocarbon group is preferably an alkylene group.

Examples of the amine represented by the formula (C3) include hexylamine, heptylamine, octylamine, nonylamine, decylamine, dibutylamine, dipentylamine, dihexylamine, diheptylamine, dioctylamine, dinonylamine, didecylamine, triethylamine, trimethylamine, tripropylamine, tributylamine, tripentylamine, trihexylamine, triheptylamine, trioctylamine, trinonylamine, tridecylamine, methyldibutylamine, methyldipentylamine, methyldihexylamine, methyldicyclohexylamine, methyldiheptylamine, methyldioctylamine, methyldinonylamine, methyldidecylamine, ethyldibutylamine, ethydipentylamine, ethyldihexylamine, ethydiheptylamine, ethyldioctylamine, ethyldinonylamine, ethyldidecylamine, dicyclohexylmethylamine, tris[2-(2-methoxyethoxy)ethyl]amine, triisopropanolamine, ethylenediamine, tetramethylenediamine, hexamethylenediamine, 4,4′-diamino-1,2-diphenylethane, 4,4′-diamino-3,3′-dimethyldiphenylmethane and 4,4′-diamino-3,3′-diethyldiphenylmethane.

Examples of the amine represented by the formula (C4) include piperazine. Examples of the amine represented by the formula (C5) include morpholine. Examples of the amine represented by the formula (C6) include piperidine and hindered amine compounds having a piperidine skeleton as disclosed in JP 11-52575 A. Examples of the amine represented by the formula (C7) include 2,2′-methylenebisaniline. Examples of the amine represented by the formula (C8) include imidazole and 4-methylimidazole. Examples of the amine represented by the formula (C9) include pyridine and 4-methylpyridine. Examples of the amine represented by the formula (C10) include di-2-pyridyl ketone, 1,2-di(2-pyridyl)ethane, 1,2-di(4-pyridyl)ethane, 1,3-di(4-pyridyl)propane, 1,2-bis(2-pyridyl)ethene, 1,2-bis(4-pyridyl)ethene, 1,2-di(4-pyridyloxy)ethane, 4,4′-dipyridyl sulfide, 4,4′-dipyridyl disulfide, 2,2′-dipyridylamine and 2,2′-dipicolylamine. Examples of the amine represented by the formula (C11) include bipyridine.

The photoresist composition of the present invention usually contains one or more solvents. Examples of the solvent include a glycol ether ester such as ethyl cellosolve acetate, methyl cellosolve acetate and propylene glycol monomethyl ether acetate; a glycol ether such as propylene glycol monomethyl ether; an acyclic ester such as ethyl lactate, butyl acetate, amyl acetate and ethyl pyruvate; a ketone such as acetone, methyl isobutyl ketone, 2-heptanone and cyclohexanone; and a cyclic ester such as γ-butyrolactone.

The amount of the solvent is usually 90% by weight or more, preferably 92% by weight or more preferably 94% by weight or more based on total amount of the photoresist composition of the present invention. The amount of the solvent is usually 99.9% by weight or less based on total amount of the photoresist composition of the present invention. The photoresist composition containing a solvent can be preferably used for producing a thin layer photoresist pattern.

The photoresist composition of the present invention can contain, if necessary, a small amount of various additives such as a sensitizer, a dissolution inhibitor, other polymers, a surfactant, a stabilizer and a dye as long as the effect of the present invention is not prevented.

A photoresist pattern can be produced by the following steps (1) to (5):

(1) a step of applying the photoresist composition of the present invention on a substrate,

(2) a step of forming a photoresist film by conducting drying,

(3) a step of exposing the photoresist film to radiation,

(4) a step of baking the exposed photoresist film, and

(5) a step of developing the baked photoresist film with an alkaline developer, thereby forming a photoresist pattern.

The applying of the photoresist composition on a substrate is usually conducted using a conventional apparatus such as spin coater.

The formation of the photoresist film is usually conducted using a heating apparatus such as hot plate or a decompressor, and the heating temperature is usually 50 to 200° C., and the operation pressure is usually 1 to 1.0*10⁵ Pa.

The photoresist film obtained is exposed to radiation using an exposure system. The exposure is usually conducted through a mask having a pattern corresponding to the desired photoresist pattern. Examples of the exposure source include a light source radiating laser light in a UV-region such as a KrF excimer laser (wavelength: 248 nm), an ArF excimer laser (wavelength: 193 nm) and a F₂ laser (wavelength: 157 nm), and a light source radiating harmonic laser light in a far UV region or a vacuum UV region by wavelength conversion of laser light from a solid laser light source (such as YAG or semiconductor laser).

The temperature of baking of the exposed photoresist film is usually 50 to 200° C., and preferably 70 to 150° C.

The development of the baked photoresist film is usualt carried out using a development apparatus. The alkaline developer used may be any one of various alkaline aqueous solution used in the art. Generally, an aqueous solution of tetramethylammonium hydroxide or (2-hydroxyethyl)trimethylammonium hydroxide (commonly known as “choline”) is often used. After development, the photoresist pattern formed is preferably washed with ultrapure water, and the remained water on the photoresist pattern and the substrate is preferably removed.

The photoresist composition of the present invention provides a photoresist pattern showing good resolution, and therefore, the photoresist composition of the present invention is suitable for ArF excimer laser lithography, KrF excimer laser lithography, ArF immersion lithography, EUV (extreme ultraviolet) lithography, EUV immersion lithography and EB (electron beam) lithography, and especially suitable for KrF excimer laser lithography, EUV lithography and EB lithography.

### EXAMPLES

The present invention will be described more specifically by Examples, which are not construed to limit the scope of the present invention.

The “%” and “part(s)” used to represent the content of any component and the amount of any material used in the following examples and comparative examples are on a weight basis unless otherwise specifically noted. The weight-average molecular weight of any material used in the following examples is a value found by gel permeation chromatography [HLC-8120GPC Type, Column (Three Columns with guard column): TSKgel Multipore HXL-M, manufactured by TOSOH CORPORATION, Solvent: Tetrahydrofuran, Flow rate: 1.0 mL/min., Detector: RI detector, Column temperature: 40° C., Injection volume: 100 μL] using standard polystyrene as a standard reference material. Structures of compounds were determined by NMR (ECA-500 Type, manufactured by JEOL LTD.) and mass spectrometry (Liquid Chromatography: 1100 Type, manufactured by AGILENT TECHNOLOGIES LTD., Mass Spectrometry: LC/MSD Type or LC/MSD TOF Type, manufactured by AGILENT TECHNOLOGIES LTD.).

### Example 1

### Synthesis of Compound Represented by the Formula (I-1)

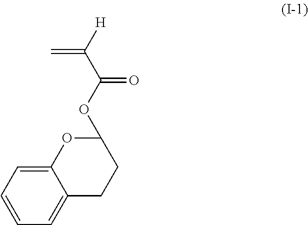

To the four-necked flask equipped with a condenser and a stirrer, 15.0 parts of 2-chromanol, 19.3 parts of triethylamine and 75 parts of tetrahydrofuran were added. The resultant mixture was cooled down to 0° C. To the mixture, 15.2 parts of methacrylic chloride was added dropwise over 15 minutes. The mixture obtained was heated up and the temperature thereof was adjusted at room temperature. The mixture was stirred for 5 hours to conduct the reaction. The reaction mixture obtained was mixed with 45 parts of aqueous saturated sodium hydrogen carbonate solution followed by extracting with 75 parts of ethyl acetate. The organic layer obtained was washed with 62 parts of aqueous saturated sodium chloride solution and then, dried over magnesium sulfate. After drying, the organic layer was concentrated to obtain 23.2 parts of clear colorless oily matter. The oily matter was purified with column chlromatography using heptane and ethyl acetate as an eluent to obtain 10.8 parts of the compound represented by the formula (I-1).

¹H-NMR (500.16 MHz, CDCl₃): δ 1.91 (s, 3H), 2.03-2.10 (m, 1H), 2.14-2.19 (m, 1H), 2.70 (d, 1H), 3.01 (t, 1H), 5.56 (q, 1H), 6.08 (d, 1H), 6.59 (t, 1H), 6.86 (d, 1H), 6.90 (t, 1H), 7.07 (d, 1H), 7.11 (t, 1H)

LS/ESIMS (+): calculated 218.094, measured 241.0 [M+Na]⁺, 459.1 [M+Na]⁺.

In the following Resin Synthesis Examples, Monomer (A), Monomer (B), Monomer (C), Monomer (D) and Monomer (E) represented by the followings were used.

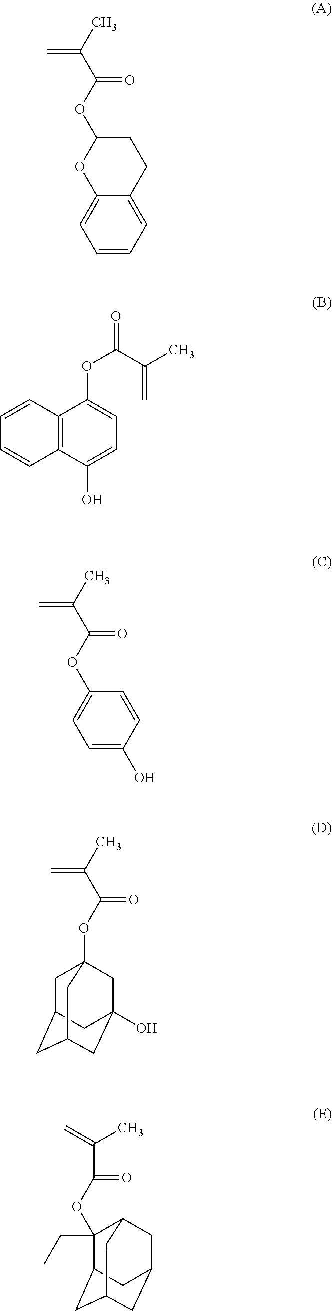

### Resin Synthesis Example 1

### Synthesis of Resin A1

To a four-necked flask equipped with a condenser and a stirrer, 6.0 parts of 1,4-dioxane was added and then, heated up to 87° C.

A solution prepared by dissolving 5.4 parts of Monomer (A), 3.5 parts of Monomer (C), 1.2 parts of Monomer (D) and 0.7 part of azobisisobutyronitrile in 9.0 parts of 1.4-dioxane was added dropwise over 1 hour thereto. The resultant mixture was stirred at 87° C. for 6 hours. The reaction mixture obtained was poured into a cooled mixture of 91 parts of methanol and 39 parts of water to cause precipitation. The resin precipitated was isolated and then, was mixed with 65 parts of methanol. The precipitated resin was isolated by filtration and then, dried under reduced pressure. As a result, 6.6 parts of a resin having a weight-average molecular weight of 9.5*10³ was obtained. The resin had the following structural units.

This is called as resin A1.

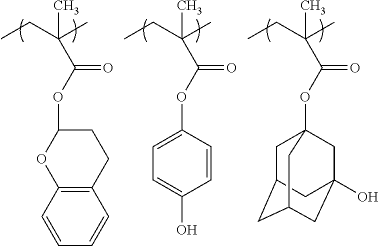

### Resin Synthesis Example 2

### Synthesis of Resin A2

To a four-necked flask equipped with a condenser and a stirrer, 7.7 parts of 1,4-dioxane was added and then, heated up to 87° C.

A solution prepared by dissolving 5.0 parts of Monomer (A), 6.5 parts of Monomer (B), 1.4 parts of Monomer (D) and 1.1 part of azobisisobutyronitrile in 11.5 parts of 1.4-dioxane and 9.8 parts of tetrahydrofuran was added dropwise over 1 hour thereto. The resultant mixture was stirred at 87° C. for 6 hours. The reaction mixture obtained was poured into a cooled mixture of 117 parts of methanol and 50 parts of water to cause precipitation. The resin precipitated was isolated and then, was mixed with 83 parts of methanol. The precipitated resin was isolated by filtration and then, dried under reduced pressure. As a result, 7.2 parts of a resin having a weight-average molecular weight of 7.6*10³ was obtained.

The resin had the following structural units. This is called as resin A2.

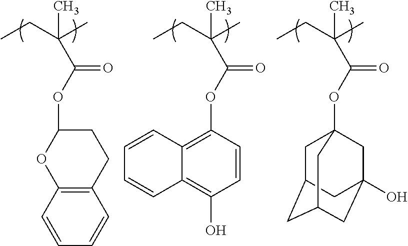

### Resin Synthesis Example 3

### Synthesis of Resin H1

To a flask, 279 parts of isopropanol, 59.6 parts of Monomer (E) and 90.8 parts of p-acetoxystyrene were added to prepare a solution, and then, the gas in the flask was replaced by nitrogen.

The solution was heated up to 75° C., and to the solution, a solution prepared by dissolving 11.05 parts of dimethyl 2,2-azobis(2-methylpropionate) in 22.11 parts of isopropanol was added dropwise. The obtained mixture was heated at 75° C. for 0.3 hour and then, refluxed for about 12 hours. The reaction mixture obtained was diluted with acetone, and then, the resultant mixture was poured into methanol to cause precipitation to obtain a copolymer.

The obtained copolymer was filtrated to obtain 250 parts of a crude copolymer. The obtained crude copolymer was mixed with 239 parts of methanol and 10.8 parts of 4-dimethylaminopyridine. The obtained mixture was refluxed for 20 hours and then, cooled. The obtained reaction mixture was neutralized with 8.0 parts of glacial acetic acid and the resultant mixture was poured into water to cause precipitation. The precipitate was isolated by filtration and then, dissolved in acetone. The obtained solution was poured into water to cause precipitation. This operation was repeated three times for purification. As a result, 102.8 parts of a polymer having a weight-average molecular weight of about 8.2*10³ was obtained.

The polymer had the following structural units. This is called as resin H1.

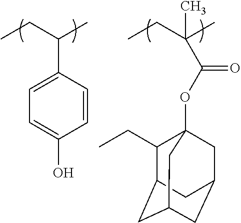

### Examples 1 to 3 and Comparative Example 1

### <Resin>

### A1: Resin A1

### A2: Resin A2

### H1: Resin H1

### <Acid Generator>

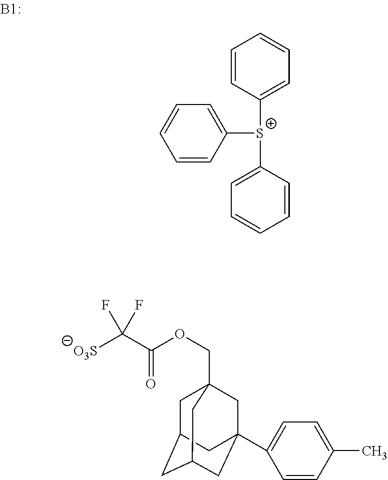

### <Quencher>

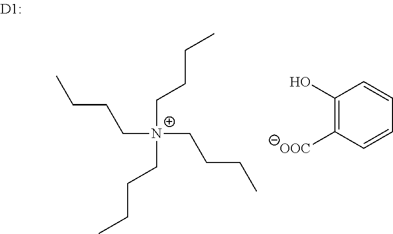

C1: 2,6-diisopropylaniline

### <Solvent>

| | | |
|---|---|---|
| Y1: | propylene glycol monomethyl ether | 420 parts |
| | propylene glycol monomethyl ether acetate | 150 parts |
| | γ-butyrolactone | 5 parts |

The following components were mixed and dissolved, further, filtrated through a fluorine resin filter having pore diameter of 0.2 μm, to prepare photoresist compositions.

Resin (kind and amount are described in Table 3)

Acid generator (kind and amount are described in Table 3)

Quencher (kind and amount are described in Table 3)

Solvent Y1

TABLE 3

| | Resin | Acid Generator | Quencher | |
|---|---|---|---|---|
| | (kind/amount | (kind/amount | (kind/amount | PB(° C.)/ |
| Ex. No. | (part)) | (part)) | (part)) | PEB(° C.) |
| Ex. 1 | A1/10 | B1/2.75 | C1/0.05 | 110/110 |
| Ex. 2 | A1/10 | B1/2.5 | D1/0.15 | 110/130 |
| Ex. 3 | A2/10 | B1/2.5 | D1/0.15 | 110/110 |
| Comp. Ex. 1 | H1/10 | B1/1.5 | C1/0.07 | 100/100 |

Silicon wafers were each contacted with hexamethyldisilazane at 90° C. for 60 seconds on a direct hot plate and each of the resist compositions prepared as above was spin-coated over the silicon wafer to give a film thickness after drying of 0.06 μm. After application of each of the photoresist compositions, the silicon wafers thus coated with the respective photoresist compositions were each prebaked on a direct hotplate at the temperature shown in the column of “PB” in Table 3 for 60 seconds. Using a writing electron beam lithography system (“HL-800D” manufactured by Hitachi, Ltd., 50 KeV), each wafer on which the respective resist film had been thus formed was exposed to a line and space pattern, while changing stepwise the exposure quantity.

After the exposure, each wafer was subjected to post-exposure baking on a hotplate at the temperature shown in the column of “PEB” in Table 3 for 60 seconds and then to paddle development with an aqueous solution of 2.38% by weight tetramethylammonium hydroxide for 60 seconds.

Each of a pattern developed on the silicon substrate after the development was observed with a scanning electron microscope, and the results of which are shown in Table 4.

Resolution: The amount of exposure that each photoresist pattern became 1:1 line and space pattern was as effective sensitivity.

When line and space pattern having 50 nm or less of the line width was developed at effective sensitivity, resolution is good and its evaluation is marked by “◯”, and when line and space pattern having 50 nm of the line width was not developed at effective sensitivity, resolution is bad and its evaluation is marked by “X”.

TABLE 4

| Ex. No. | Resolution |
|---|---|
| Ex. 1 | ◯ |
| Ex. 2 | ◯ |
| Ex. 3 | ◯ |
| Comp. Ex. 1 | X |

### Example 4

A resist pattern can be obtained according to the same manner as described in Example 1, except that an EUV stepper is used in place of the writing electron beam lithography system.

### Example 5

A resist pattern can be obtained according to the same manner as described in Example 2, except that an EUV stepper is used in place of the writing electron beam lithography system.

### Example 6

A resist pattern can be obtained according to the same manner as described in Example 2, except that an EUV stepper is used in place of the writing electron beam lithography system.

The present photoresist composition provides a good resist pattern having good resolution, and is especially suitable for KrF excimer laser lithography, EUV lithography and EB lithography.

What is claimed is:

1. A compound represented by the formula (I): 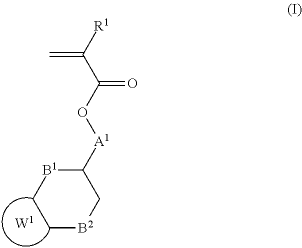
wherein R¹ represents a hydrogen atom or a methyl group, A¹ represents a single bond or *—(CH₂)ₘ—CO—O— in which m represents an integer of 1 to 4 and * represents a binding position to —O—, B¹ represents —O— or —S—, B² represents —CH₂—, —O— or —S— and W¹ represents an optionally substituted aromatic ring.

2. A resin comprising a structural unit derived from the compound according to claim 1.

3. A photoresist composition comprising the resin according to claim 2.

4. The photoresist composition according to claim 3, wherein the photoresist composition further contains an acid generator.

5. The photoresist composition according to claim 3 or 4, wherein the photoresist composition further contains a basic compound.

6. A process for producing a photoresist pattern comprising the following steps (1) to (5):
(1) a step of applying the photoresist composition according to any one of claims 3 to 5 on a substrate,
(2) a step of forming a photoresist film by conducting drying,
(3) a step of exposing the photoresist film to radiation,
(4) a step of baking the exposed photoresist film, and
(5) a step of developing the baked photoresist film with an alkaline developer, thereby forming a photoresist pattern.
